(19) United States
(12) Patent Application Publication
(10) Pub. No.: US 2015/0324548 A1
(43) Pub. Date: Nov. 12, 2015

(54) MEDICATION DELIVERY SYSTEM

(71) Applicant: Jeffrey Scott Eder, Mill Creek, WA (US)

(72) Inventor: Jeffrey Scott Eder, Mill Creek, WA (US)

(73) Assignee: KANTRACK LLC, Redmond, WA (US)

(21) Appl. No.: 14/803,401

(22) Filed: Jul. 20, 2015

Related U.S. Application Data
(63) Continuation of application No. 14/436,883, filed on Apr. 18, 2015.
(60) Provisional application No. 61/756,409, filed on Jan. 24, 2013.

(51) Int. Cl. G06F 19/00; A61M 5/172; A61M 5/142

(52) U.S. Cl. CPC G06F 19/3456; G06F 19/3437; A61M 5/142; A61M 5/1723; A61M 2205/502; A61M 2205/6018; A61M 2205/52; A61M 2205/3584

(57) ABSTRACT

This disclosure comprises a system, non-transitory computer readable storage medium and method for delivering medications and/or medical treatments that are appropriate to the resilient context of an individual patient. The resilient context comprises a predictive model for each of one or more patient function measures and a predictive model of patient resilience where said models are all developed by learning from the data associated with the individual patient. The medical advice, medical diagnoses and/or medical treatments may be provided “as is” and/or they may be customized to match the specific resilient context of the individual patient.

### CROSS-REFERENCE TO RELATED APPLICATION

This is a continuation of U.S. patent application Ser. No. 14/436,883 filed May 26, 2015, which is a U.S. national stage entry of International Application No. PCT/US2013/031020 entitled “Individualized Medicine System” filed Mar. 13, 2013 the disclosure of which is incorporated herein by reference in its entirety for all purposes. PCT/US2013/031020 was published as WO/2014/116276 on Jul. 31, 2014 and claims the benefit of U.S. Provisional Patent Application No. 61/756,409 filed Jan. 24, 2013, the disclosure of which is also incorporated herein by reference in its entirety for all purposes.

### BACKGROUND

A method, computer program product and system for developing and/or providing medical advice, medical diagnoses and/or medical treatments that are appropriate to the resilient context of an individual patient. The resilient context comprises a predictive model for each of one or more patient function measures and a predictive model of patient resilience where said models are all developed by learning from the data associated with the individual patient.

### SUMMARY OF THE INVENTION

This disclosure comprises a method, computer program product and system for developing and/or providing medical advice, medical diagnoses and/or medical treatments that are appropriate to the resilient context of a subject entity (22). The system incorporates a non-transitory computer program product to manage the completion of the required processing by one or more processors in a computer system. The medical advice, diagnoses and/or treatments may be provided “as is” and/or they may be individualized to match a specific resilient context of the subject entity (22).

It is a general object of the embodiment of the invention described herein to provide a novel and useful system for developing, identifying and/or providing medical advice, medical diagnoses and/or medical treatments (hereinafter, individualized medicine services) that are appropriate to the resilient context of the subject entity (22).

The data regarding the resilient context of the subject entity (22) are continuously analyzed and updated using an Entity Resilience System (30). The Entity Resilience System (30), in turn communicates with a number of other systems as required to support the development and delivery of individualized medical services to the subject entity (22).

### BRIEF DESCRIPTION OF DRAWINGS

These and other objects, features and advantages will be more readily apparent from the following description of the one embodiment in which:

FIG. 1 is a software block diagram showing components of the Individualized Medicine System (100);

FIG. 2 is a software block diagram of an implementation of the Individualized Medicine System (100) described herein;

FIG. 3 is a diagram showing the data windows that are used for receiving information from and transmitting information;

FIG. 4 is a diagram showing the tables in the application database (51) described herein that are utilized for data storage during the processing in the innovative Individualized Medicine System (100);

FIG. 5A and FIG. 5B are software block diagrams showing the sequence of steps in the present embodiment used for operating the Individualized Medicine System (100) and managing medical equipment (8) operation;

FIG. 6 is a software block diagram showing processing steps of the Entity Resilience System (30);

FIG. 7A and FIG. 7B are block diagrams showing a relationship between actions, elements, events, factors, locations, measures, transactions and entity mission for an entity (920) and for an extended entity (950);

FIG. 8 shows a summary of risks and a resilience index for a measure/scenario combination;

FIG. 9 is a diagram showing the tables in the Resilient Contextbase (50) of the present embodiment that are utilized for data storage during the Entity Resilience System (30) processing;

FIG. 10 is a diagram of an implementation of the Entity Resilience System (30);

FIG. 11A, FIG. 11B, FIG. 11C and FIG. 11D are block diagrams showing the sequence of steps in the present embodiment used for specifying system settings, preparing data for processing and specifying the subject entity (22) measures;

FIG. 12A, FIG. 12B, FIG. 12C, FIG. 12D and FIG. 12E are block diagrams showing the sequence of steps in the present embodiment used for creating a Resilient Contextbase (50) for a subject entity (22);

FIG. 13A and FIG. 13B are block diagrams showing the sequence in steps in the present embodiment used in providing a plurality of Resilient Context Services, programming bots and producing performance reports;

FIG. 14 is a software block diagram showing the sequence of processing steps in the present embodiment used for receiving and transmitting data through a resilient context interface window (711);

FIG. 15 is a diagram showing how the Entity Resilience System (30) develops and supports a natural language interface window (714) and associated processing;

FIG. 16 is a sample report showing the efficient frontier and resilient frontier for Entity A and the current position of Entity A relative to the efficient frontier and the resilient frontier;

FIG. 17 shows some of the training methods used by the Entity Resilience System (30) used in developing models by learning from the data;

FIG. 18 shows a universal resilient context specification format;

FIG. 19 provides an overview of the order of simulation by level for an extended subject entity; and

FIG. 20 shows the default function measures for the subject entity (22) and subject entity systems, organs and cells.

### DETAILED DESCRIPTION

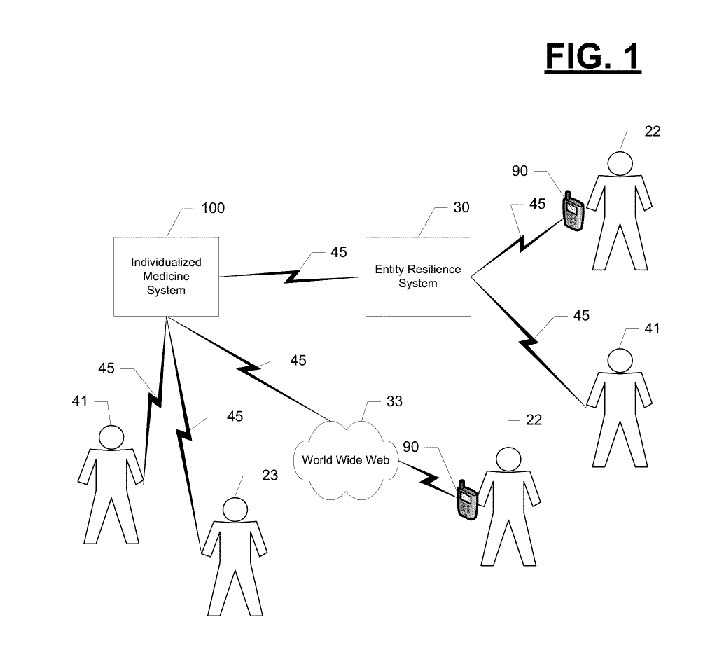

FIG. 1 provides an overview of the systems that comprise the Individualized Medicine System (100). The Individualized Medicine System (100) is used for identifying, developing and providing individualized medicine services that are appropriate to the resilient context of a specific subject entity (22). In accordance with the present embodiment, the starting point for processing is the Entity Resilience System (30) that identifies the current resilient context for the subject entity (22) using as many as eight of the primary layers (or aspects) of resilient context.

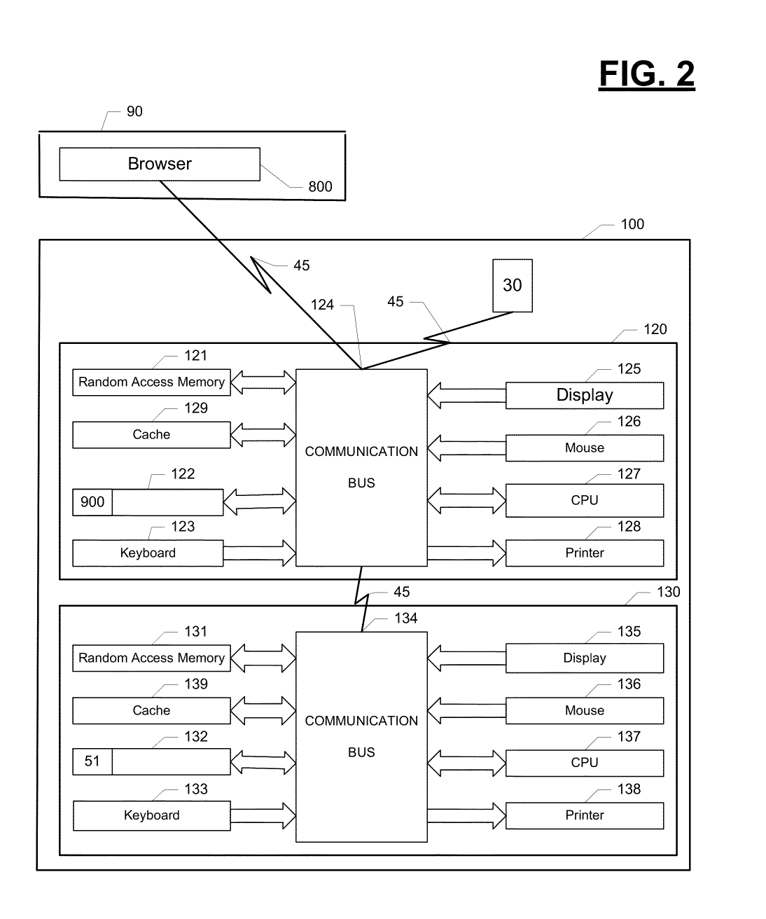

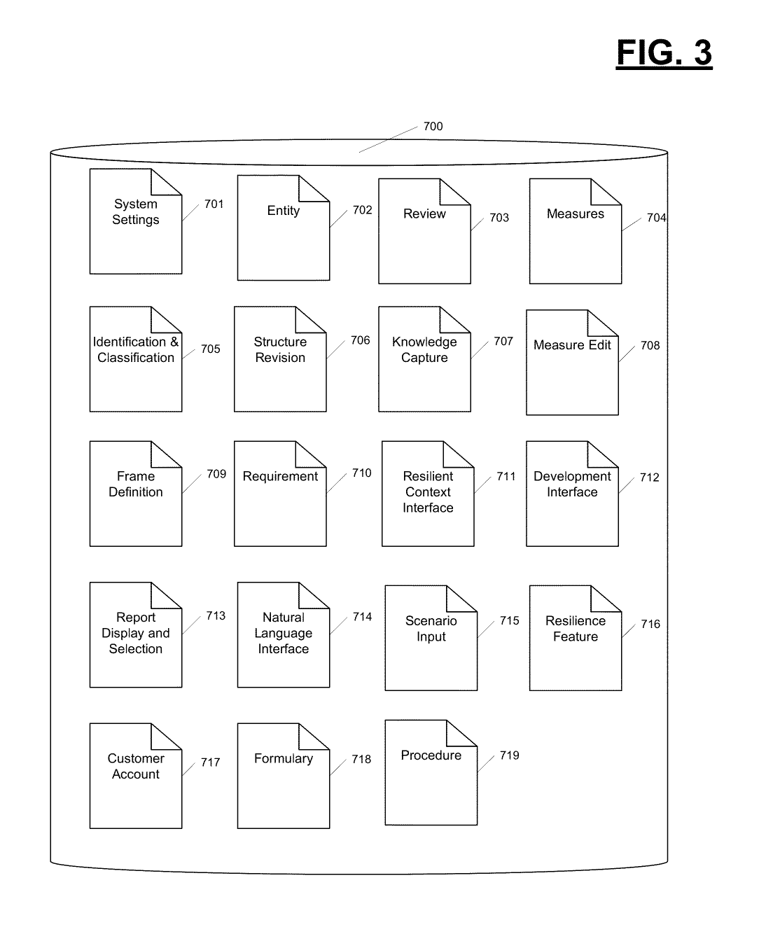

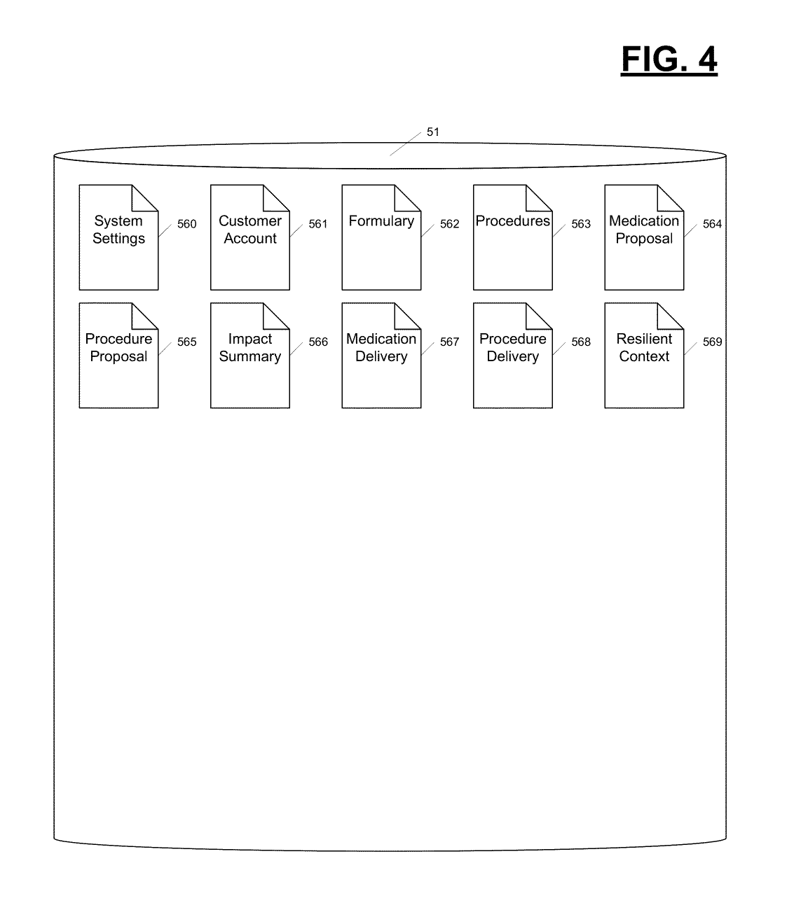

In one embodiment, the Individualized Medicine System (100) is comprised of two computers (120, 130), an application database (51) and a network connection to at least one Entity Resilience System (30). As shown in FIG. 2, one embodiment of the two computers is a user-interface personal computer (120) connected to a database-server computer (130) via a network (45). The user interface personal computer (120) is also connected via the network (45) to an internet access device (90) such as a computer, tablet or a smartphone that contains browser software (800) such as Chrome, Internet Explorer or Mozilla Firefox. While only one instance of an Entity Resilience System (30) is shown, it is to be understood that the system may interface with an Entity Resilience System (30) for more than one entity.

The user-interface personal computer (120) has a read/write random access memory (121), a hard drive (122) for storage of a subject data table and the Individualized Medicine Input Output System (50), a keyboard (123), a communication bus containing all adapters and bridges (124), a display (125), a mouse (126), a CPU (127) and a printer (128). The database-server computer (130) has a read/write random access memory (131), a hard drive (132) for storage of the application database (51), a keyboard (133), a communication bus card containing all adapters and bridges (134), a display (135), a mouse (136), a CPU (137) and a printer (138).

Again, it is to be understood that the diagram of FIG. 2 is merely illustrative of one embodiment. For example, it should be understood that using a computer with one or more graphics processing units (GPU's) may speed the processing described herein. In a similar manner a user (41) and/or the subject entity (22) could interface directly with one or more of the computers in the system (100) instead of using an internet access device (90) with a browser (800) as described in the one embodiment.

An individualized medicine software (900) controls the performance of the central processing unit (137) as it completes the data processing used for developing and/or providing medical advice, medical diagnoses and/or medical treatments that are appropriate to the resilient context of the subject entity (22). In the embodiment illustrated herein, the software program (900) is written in a combination of C++ and Java although other languages can be used to the same effect. The subject entity (22), and user (41) can optionally interact with the application software (900) using the browser software (800) in the internet access device (90) to provide information to the application software (900) for use in completing one or more of the steps in processing.

The computers (120 and 130) shown in FIG. 2 illustratively are personal computers. Those of average skill in the art will recognize that other computing devices, such as more powerful computers (such as workstations or mainframe computers) or virtual or cloud-based computer systems (such as Amazon Cloud and/or Open Stack Cloud offerings) could also be used to perform one or more of the computer processing steps or functions described herein.

Using the systems described above, data generated by the Entity Resilience System (30) for a specific subject entity (22) may be combined with data from other sources, such as the World Wide Web (33), one or more external databases and data from one or more medical service providers (23) in the Individualized Medicine System (100). Said data are then analyzed as required to provide medical advice, medical diagnoses and/or medical treatments. As is well known in the art, data from the World Wide Web (33) and from external databases may include one or more data streams.

### Entity Resilience System

The Entity Resilience System (30) enables and supports the operation of the Individualized Medicine System (100), by providing a Resilient Context Suite of services (625) and optionally providing a plurality of Resilient Context Bots (650) and/or a Resilient Context Programming System (610). The Entity Resilience System (30) supports the development and integration of any combination of data, information and knowledge from systems that analyze, monitor, support and/or are associated with one or more subject entities (22) from three distinct areas: a social environment area (1000), a natural environment area (2000) and a physical environment area (3000). Each of these three areas can be further subdivided into domains. Each domain can in turn be divided into a hierarchy or group. Each member of a hierarchy or group is a type of entity.

The social environment area (1000) includes a political domain hierarchy (1100), a habitat domain hierarchy (1200), an interpersonal domain group (1300), a market domain hierarchy (1400) and a physical organization domain hierarchy (1500). The political domain hierarchy (1100) includes a voter entity type (1101), a precinct entity type (1102), a caucus entity type (1103), a city entity type (1104), a county entity type (1105), a state/province entity type (1106), a regional entity type (1107), a national entity type (1108), a multi-national entity type (1109) and a global entity type (1110). The habitat domain hierarchy includes a household entity type (1202), a neighborhood entity type (1203), a community entity type (1204), a city entity type (1205) and a region entity type (1206). The interpersonal domain group (1300) includes an individual entity type (1301), a nuclear family entity type (1302), an extended family entity type (1303), a clan entity type (1304), an ethnic group entity type (1305), a neighbor's entity type (1306) and a friend's entity type (1307). The market domain hierarchy (1400) includes a multi entity type (1402), an industry entity type (1403), a market entity type (1404) and an economy entity type (1405). The physical organization domain hierarchy (1500) includes a team entity type (1502), a group entity type (1503), a department entity type (1504), a division entity type (1505), a company entity type (1506) and a multi company organization entity type (1507).

The natural environment area (2000) includes a biology domain hierarchy (2100), a cellular domain hierarchy (2200), an organism domain hierarchy (2300) and a protein domain hierarchy (2400). The biology domain hierarchy (2100) contains a species entity type (2101), a genus entity type (2102), a family entity type (2103), an order entity type (2104), a class entity type (2105), a phylum entity type (2106) and a kingdom entity type (2107). The cellular domain hierarchy (2200) includes a macromolecular complexes entity type (2202), a protein entity type (2203), a RNA entity type (2204), a DNA entity type (2205), a methylation entity type (2206), an organelles entity type (2207) and cells entity type (2208). The organism domain hierarchy (2300) contains a cell entity type (2301), an organs entity type (2302), a system (e.g., circulatory, endocrine, nervous, etc.) entity type (2303) and an organism entity type (2304). The protein domain hierarchy contains a monomer entity type (2400), a dimer entity type (2401), a large oligomer entity type (2402), an aggregate entity type (2403) and a particle entity type (2404).

The physical environment area (3000) contains a chemistry group (3100), a geology domain hierarchy (3200), a physics domain hierarchy (3300), a space domain hierarchy (3400), a tangible goods domain hierarchy (3500), a water group (3600) and a weather group (3700). The chemistry group (3100) contains a molecules entity type (3101), a compounds entity type (3102), a chemicals entity type (3103) and a catalysts entity type (3104). The geology domain hierarchy (3200) contains a minerals entity type (3202), a sediment entity type (3203), a rock entity type (3204), a landform entity type (3205), a plate entity type (3206), a continent entity type (3207) and a planet entity type (3208). The physics domain hierarchy (3300) contains a quark entity type (3301), a particle zoo entity type (3302), a protons entity type (3303), a neutrons entity type (3304), an electrons entity type (3305), an atoms entity type (3306), and a molecules entity type (3307). The space domain hierarchy (3400) contains an asteroids entity type (3403), a comets entity type (3404), a planets entity type (3405), a stars entity type (3406), a solar system entity type (3407), a galaxy entity type (3408) and universe entity type (3409). The tangible goods hierarchy (3500) contains a money entity type (3501), a compounds entity type (3502), a minerals entity type (3503), a components entity type (3504), a subassemblies entity type (3505), an assembly's entity type (3506), a subsystems entity type (3507), a goods entity type (3508) and a systems entity type (3509). The water group (3600) contains a pond entity type (3602), a lake entity type (3603), a bay entity type (3604), a sea entity type (3605), an ocean entity type (3606), a creek entity type (3607), a stream entity type (3608), a river entity type (3609) and a current entity type (3610). The weather group (3700) contains an atmosphere entity type (3701), a clouds entity type (3702), a lightning entity type (3703), a precipitation entity type (3704), a storm entity type (3705) and a wind entity type (3706).

Individual entities are items of one or more entity type. Entities and subject entities (22) can also be linked together to follow a chain of events that impacts one or more subjects and/or entities. These chains can be recursive. The domain hierarchies can be organized into different categories and they can also be expanded, modified, extended or pruned in order to support different analyses.

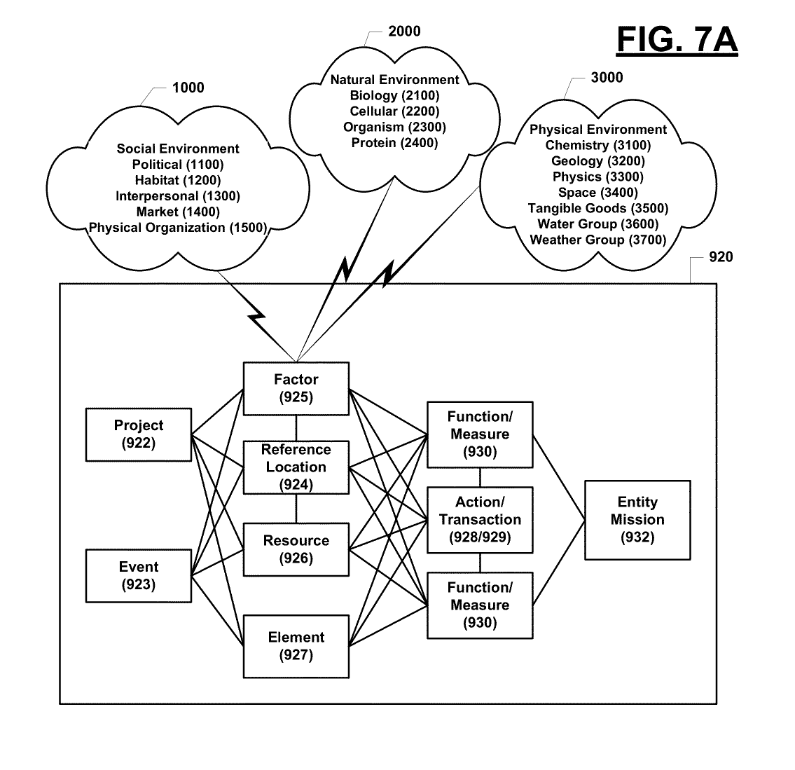

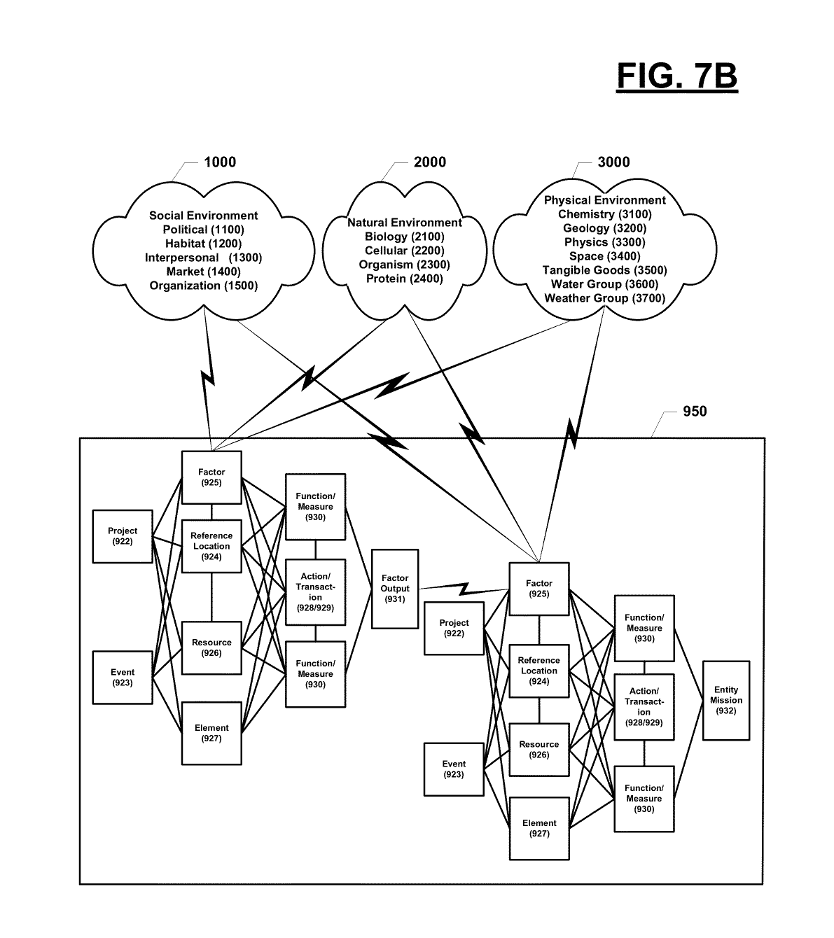

Data, information and knowledge from these different domains can be integrated and analyzed in order to support the creation of one or more resilient contexts for the subject entity (22). The one or more resilient contexts developed by this system focus on a mission of the single subject entity (22) as shown in FIG. 7A and/or an extended subject entity (950) as shown in FIG. 7B. FIG. 7A shows a block diagram for the subject entity (920) that contains a block for: a project (922), an event (923), a reference location (924), a factor (925), a resource (926), an element (927) and an action/transaction (928/929). The block diagram also shows a plurality of function measures (930) and an entity mission (932).

In some embodiments, the default mission is maintaining subject entity health which is measured using a defined measure, such as, for example, Quality of Well-Being (QWB). Accordingly, the default entity mission is to maintain or improve QWB levels. The QWB measure evaluates mobility, physical activity, and social activity so the default function measures are measures of mobility, physical activity and social activity. In other embodiments, different health care related measures may be used as the entity mission. For example, various quality of life measures are used for measuring various aspects of an individual's state.

While the block diagram only shows a single item in each block, it is to be understood that the system of the present embodiment can support the analysis and management of entity resilience when there are a plurality of items for each aspect of resilient context. For example, the subject entity (22) function measure 930 and mission 932 may be impacted by a plurality of projects, a plurality of events, a plurality of factors, a plurality of resources, a plurality of actions and a plurality of transactions and a plurality of elements in a plurality of locations.

FIG. 7B shows a block diagram for an extended entity (950) that contains a block for: a project (922), an event (923), a reference location (924), a factor (925), a resource (926), an element (927), an action/transaction (928/929) and a block diagram for a factor output (931). While the block diagram only shows a single item in each block, it is to be understood that the system of the present embodiment can support the analysis and management of entity resilience when there are a plurality of items for each aspect of resilient context. For example the subject entity (22) function measure performance and mission for the extended subject entity may be impacted by a plurality of projects, a plurality of events, a plurality of factors, a plurality of resources, a plurality of actions and a plurality of transactions and a plurality of elements in a plurality of locations. While FIG. 7B shows a separate block diagram for only one factor output in the extended entity (950). It is to be understood that the number of components of resilient context (elements, factors and/or resources) that are modeled with separate block diagrams is determined by the contribution and entity depth cutoffs established by the user (41) in the system settings.

After one or more resilient contexts are developed for the subject entity (22), they can be combined, reviewed, analyzed and/or applied using one or more of the resilient context-aware services in a Resilient Context Suite (625) of services. These services are optionally modified to meet subject entity (22) requirements using a Resilient Context Programming System (610). The Resilient Context Programming System (610) also supports the maintenance of the services in the Resilient Context Suite (625), the creation of newly defined stand-alone services, the development of new services and/or the programming of resilient context-aware bots. The system of the present embodiment systematically develops the one or more resilient contexts for distribution in an Entity Resilience System (30). These resilient contexts are in turn used to support the comprehensive analysis of subject entity (22) performance, develop one or more shared contexts to support collaboration, simulate subject entity (22) performance and/or turn data into knowledge. Processing by the Entity Resilience System (30) may be completed in three steps:
1. Subject entity (22) definition and data preparation;
2. Resilient context and Resilient Contextbase (50) development, and
3. Resilient Context Service deployment.

The first processing step in the Entity Resilience System (30) defines the subject entity (22) that will be modeled, prepares the data from one or more sources, such as devices (3), entity narrow system databases (5), partner narrow system databases (6), external databases (7), the World Wide Web (33), external services (9) and/or the Resilient Context Input System (601) for processing and then uses these data to specify subject entity (22) functions and measures. As is well known in the art, data from the World Wide Web (33) and external services (9) includes streaming data that can be incorporated as data sources in place of and/or as a supplement to one or more databases.

As part of the first stage of processing, the user (41) identifies the subject entity (22) by using existing hierarchies and groups, adding a new hierarchy or group or modifying the existing hierarchies and/or groups in order to fully define the subject. For example, a white blood cell entity is an item with the cell entity type (2208) and an element of the circulatory system and auto-immune system (2303). In a similar fashion, entity Jane Doe could be an item within the organism entity type (2300), an element of a nuclear family entity (1402), an element of an extended family entity (1403) and/or an element of a household entity (1202). This individual would be expected to have one or more functions and measures for each entity type she is associated with. Separate systems that tried to analyze the five different roles of the individual in each of the five hierarchies would probably save some of the same data five separate times and use the same data in five different ways. At the same time, all of the work to create these five separate systems might provide very little insight because the resilient context for measure performance of this subject entity (22) at any one period is a blend of the resilient context associated with each of the five different functions she is simultaneously performing in the different domains. Predefined templates for the different entity types can be used at this point to facilitate the specification of the subject entity (22) (these same templates can be used to accelerate learning by the system of the present embodiment). This specification can include an identification of other subjects that are related to the entity. For example, the specification for an individual could identity her friends, family, home, place of work, church, car, typical foods, hobbies, favorite malls, etc. using one of these predefined templates. These definitions can be supplemented by identifying actions, elements, events, factors, processes, projects, risks and resources that impact the subject. After the subject entity (22) definition is completed, structured data and information, transaction data and information, descriptive data and information, unstructured data and information, text data and information, geo-spatial data and information, image data and information, array data and information, web data and information, video data and video information, device data and information, and/or service data and information are made available for analysis by converting data formats before mapping these data to a Resilient Contextbase (50) in accordance with a common schema or ontology that is based on the subject definition provided by the user (41) and the pre-defined hierarchies or templates.

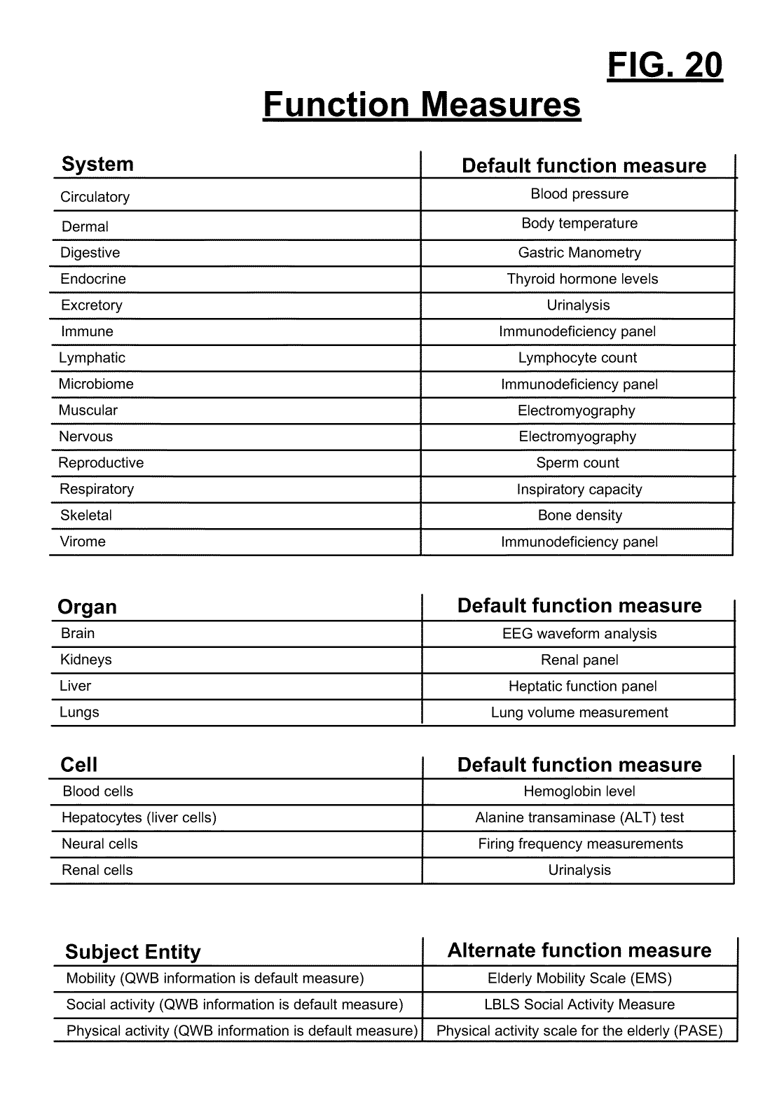

In one embodiment the common schema would be implemented by associating each piece of data with at least one descriptor, such as a tag in accordance with the criteria shown below:
Tag 1ac-Subject entity characteristics (e.g., individual patient name, occupation, age and weight);
Tab 1am—Subject entity function measurements (e.g., quality of well being measure, measures of mobility, physical activity, and social activity);
Tag 1bc-Subject entity system characteristics (e.g., circulatory, dermal, digestive, endocrine, excretory, immune, lymphatic, microbiome—enterotype, muscular, nervous, reproductive, respiratory, skeletal or virome systems);
Tag 1bm-Subject entity system function measures (see FIG. 20);
Tag 1cc—Subject entity organ characteristics by system (e.g., the circulatory system includes the heart and the blood vessels; the dermal system includes the skin, hair, and nails; the digestive system includes the mouth, the pharynx, the esophagus, the stomach, the liver, the gall bladder, the pancreas, the small intestine, the large intestine, the rectum, and the anus; the endocrine system includes all of the glands in the subject entity's body; the excretory system includes the skin, the lungs, the liver, the kidneys, and the large intestine; the microbiome includes the totality of microbes that reside within or on the subject entity; the muscular system includes all of the muscles and tendons of the subject entity's body; the nervous system includes the brain, the spinal cord, and all of the nerves of the subject entity's body; the reproductive system mainly includes the testes and the penis in men and the ovaries and the uterus in women; the respiratory system includes the nose, the mouth, the pharynx, the larynx, the trachea, the bronchial tubes, and the lungs; the skeletal system includes all of the bones, joints, ligaments, and tendons of the subject entity's body; and the virome all the viruses that inhabit the subject entity);
Tag 1cm—Subject entity organ function measures by system (see FIG. 20 for some examples);
Tag 1dc—Cell characteristics by subject entity organ or system (e.g., blood cells in the circulatory system; skin cells in the dermal system; t-cells in the immune system; bacterial cells within microbiome; etc.);
Tag 1dm—Cell function measures by subject entity organ or system (see FIG. 20 for some examples);
Tag 1ec—Genetic material characteristics within the cells within each subject entity organ or system (e.g., motifs, gene clusters, genes, etc.);
Tag 1em—Genetic material function measures within the cells within each subject entity organ or system (e.g., motifs, gene clusters, genes, etc.);
Tag 1fc—Non biological subject entity related element characteristics (e.g., boat, car, house, phone, tablet, etc.);
Tag 1fm—Non biological subject entity related element function measures (e.g., boat, car, house, phone, tablet, etc.);
Tag 2c—Resource entity characteristic data;
Tag 2m—Resource entity function measure data;
Tag 3ac—Environmental entity characteristic data;
Tag 3am—Environmental entity function measure data;
Tag 3b—Event data
Tag 4—Reference frame data
Tag 5—Transaction data

In accordance with the schema shown above, all entity data are tagged with at least one tag from the group consisting of 1ac, 1am, 1bc, 1bm, 1cc, 1cm, 1dc, 1dm, 1ec, 1em, 1fc, 1fm, 2c, 2m, 3ac and 3am. Reference frame data identifies a location relative to a defined location framework (e.g., location coordinates from a Global Positioning System). Tags for reference frame designations can be applied to data for any entity or event. Transactions are defined as exchanges of elements or resources between two or more entities so the tags used previously can be used to define any transaction and the location of said transaction. Standard function measures for each subject entity system, organ, cell and genetic material are incorporated in the one embodiment. The user (41) is given the option to change said measures as part of normal processing.

The automated conversion and mapping of data and information from the existing devices (3) narrow computer-based system databases (5 & 6), external databases (7), the World Wide Web (33) and external services (9) to the common schema or ontology significantly increases the scale and scope of the analyses that can be completed by users. This innovation also gives users (41) the option to extend the life of their existing narrow systems (4) that would otherwise become obsolete. The uncertainty associated with the data from the different systems is evaluated at the time of integration.

The exact type of analyses completed by the present embodiment is defined by the entity depth selected by the user (41) For example, if the user (41) established an entity depth cutoff of 1, then the subject entity systems are modeled with separate diagrams and models. To further illustrate the flexibility of the present embodiment, if the user (41) established an entity depth cutoff of 2, then the systems and organs that contribute to the default measures of mobility, physical activity, and social activity are modeled with separate diagrams and models. Table 1 shows the relationship between the node depth specified by the user and the types of analyses that are completed.

TABLE 1

| Node depth | Type of analyses |
|---|---|
| 1 | Analysis of the impact of subject entity's systems on subject entity function measures* |
| 2 | Analysis of the impact of subject entity's systems on subject entity function measures* |
| | and analysis of impact of subject entity's organs on subject entity system function |
| | measures |
| 3 | Analysis of the impact of subject entity's systems on subject entity function measures* |
| | and analysis of impact of subject entity's organs on subject entity system function |
| | measures; and analysis of impact of different cell types on subject entity's organ function |
| | measures |
| 4 | Analysis of the impact of subject entity's systems on subject entity function measures* |
| | and analysis of impact of subject entity's organs on subject entity system function |
| | measures; analysis of impact of different cell types on subject entity's organ function |
| | measures and analysis of impact of genetic material on subject entity's cell function |
| | measures |

*(default subject entity function measures are measures of mobility, physical activity, and social activity)

In various embodiments, the Entity Resilience System (30) may also be capable of operating without completing some or all narrow system database (5 & 6) conversions and integrations as it can directly accept data that comply with the common schema or ontology. The Entity Resilience System (30) may also be capable of operating without any input from narrow systems (4). For example, the Resilient Context Input Service (601) is fully capable of providing all data directly to the Entity Resilience System (30).

The term “common schema or ontology” refers to the fact that the schema or ontology used to guide data integration can be used by all services in the Resilient Context Suite (625) of services. In short, the schema or ontology is “common” to all of the services in the Suite (625). The Entity Resilience System (30) supports the preparation and use of data, information and/or knowledge from the “narrow” systems (4) listed in Tables 2, 3, 4 and 5 and devices (3) listed in Table 6.

TABLE 2

| | |
|---|---|
| Biomedical | affinity chip analyzer, array systems, Bina box, biochip systems, bioinformatic |
| Systems | systems, biological simulation systems, blood chemistry systems, blood pressure |
| | systems, body sensors, clinical management systems, diagnostic imaging systems, |
| | electronic subject entity record systems, electrophoresis systems, electronic |
| | medication management systems, enterotype systems, enterprise appointment |
| | scheduling, enterprise practice management, evolutionary conservation data systems |
| | (both alignment-based and alignment-free), fluorescence systems, formulary |
| | management systems, functional genomic systems, galvanic skin sensors, |
| | gastrointestinal diagnostic systems, gene chip analysis systems, gene expression |
| | analysis systems, gene sequencers, glucose test equipment, high throughput |
| | screening systems (also referred to as next generation sequencing systems), immune |
| | system (e.g., t-cell) profile development systems, immunosignaturing systems, |
| | information based medical systems, laboratory information management systems, |
| | liquid chromatography, mass spectrometer systems, microarray systems, microbial |
| | signature systems, medical testing systems, microfluidic systems, molecular |
| | diagnostic systems, nanopore sequencing, nano-string systems, nano-wire systems, |
| | paper based diagnostic systems with readers, peptide mapping systems, |
| | pharmacoeconomic systems, pharmacogenomic data systems, pharmacy |
| | management systems, phylochip systems, practice management systems, protein |
| | biochip analysis systems, protein mining systems, protein modeling systems, protein |
| | sedimentation systems, protein sequencer, protein visualization systems, proteomic |
| | data systems, ribosome profiling systems, stentennas, structural biology systems, |
| | systems biology applications, tilted microarray systems, universal serial bus genome |
| | sequencer, verbal autopsy systems, methylation analysis systems, phosphoryation |
| | analysis systems |

TABLE 3

| | |
|---|---|
| Personal | appliance management systems, automobile management systems (e.g., driverless |
| Systems | car systems), blogs, contact management applications, credit monitoring systems, gps |
| | applications, home management systems, image archiving applications, image |
| | management applications, folksonomies, lifeblogs, media archiving applications, |
| | media applications, media management applications, personal finance applications, |
| | personal productivity applications (word processing, spreadsheet, presentation, etc.), |
| | personal database applications, personal and group scheduling applications, social |
| | networking applications, tags, video applications |

TABLE 4

| | |
|---|---|
| Scientific | accelerometers, atmospheric survey systems, geological survey systems, ocean |
| Systems | sensor systems, seismographic systems, sensors, sensor grids, sensor networks, |
| | smart dust |

TABLE 5

| | |
|---|---|
| Management | accounting systems**, advanced financial systems, alliance management systems, |
| Systems | asset and liability management systems, asset management systems, battlefield |
| | systems, behavioral risk management systems, benefits administration systems, |
| | brand management systems, budgeting/financial planning systems, building |
| | management systems, business intelligence systems, call management systems, |
| | cash management systems, channel management systems, claims management |
| | systems, command systems, commodity risk management systems, content |
| | management systems, contract management systems, credit-risk management |
| | systems, customer relationship management systems, data integration systems, data |
| | mining systems, demand chain systems, decision support systems, device |
| | management systems document management systems, email management systems, |
| | employee relationship management systems, energy risk management systems, |
| | expense report processing systems, fleet management systems, foreign exchange |
| | risk management systems, fraud management systems, freight management systems, |
| | geological survey systems, human capital management systems, human resource |
| | management systems, incentive management systems, information lifecycle |
| | management systems, information technology management systems, innovation |
| | management systems, instant messaging systems, insurance management systems, |
| | intellectual property management systems, intelligent storage systems, interest rate |
| | risk management systems, investor relationship management systems, knowledge |
| | management systems, litigation tracking systems, location management systems, |
| | maintenance management systems, manufacturing execution systems, material |
| | requirement planning systems, metrics creation system, online analytical processing |
| | systems, ontology systems, partner relationship management systems, payroll |
| | systems, pension systems, performance dashboards, performance management |
| | systems, price optimization systems, private exchanges, process management |
| | systems, product life-cycle management systems, project management systems, |
| | project portfolio management systems, revenue management systems, risk |
| | management information systems, sales force automation systems, scorecard |
| | systems, sensors (includes RFID), sensor grids (includes RFID), service management |
| | systems, simulation systems, six-sigma quality management systems, shop floor |
| | control systems, strategic planning systems, supply chain systems, supplier |
| | relationship management systems, support chain systems, system management |
| | applications, taxonomy systems, technology chain systems, treasury management |
| | systems, underwriting systems, unstructured data management systems, visitor (web |
| | site) relationship management systems, weather risk management systems, |
| | workforce wellness systems, workforce management systems, yield management |
| | systems and combinations thereof |

**these typically include an accounts payable system, accounts receivable system, inventory system, invoicing system, payroll system and purchasing system

TABLE 6

| | |
|---|---|
| Devices | personal digital assistants, phones, watches, clocks, lab equipment, personal |
| | computers, televisions, radios, personal fabricators, personal health monitors, |
| | refrigerators, washers, dryers, ovens, lighting controls, alarm systems, security |
| | systems, hvac systems, gps devices, smart clothes (articles of clothing that sense, |
| | record and transmit body measurements), personal biomedical monitoring devices, |
| | tablets, personal computers |

After data conversions have been identified, the user (41) may be asked to optionally specify entity functions. As mentioned previously, mobility, physical activity, and social activity are the default functions.

After the data acquisition and integration, subject entity (22) definition and measure specification are completed, processing advances to the second stage where the data are transformed into models of one or more measures, one or more context layers and a resilient context for each function measure and node combination. These models, context layers and resilient contexts are then stored in a Resilient Contextbase (50). The resilient context for the subject entity (22) can be divided into eight resilient context layers. In accordance with embodiments of the present invention, eight layers of a resilient context are:

Layer 1: A layer that defines and describes the element context over time. For example, widgets (elements) built (an action) using a new design (an element) with an automated lathe (another element) are stored in a warehouse (another element). The lathe (element) was recently refurbished (completed action) and produces 100 widgets per 8 hour shift (element characteristic). Production can be increased to 120 widgets per 8 hour shift if complete numerical control (a feature option) is added. This layer may be subdivided into any number of sub-layers along user specified dimensions such as elements of value, processes, agents, assets and combinations thereof.

Layer 2: A layer that defines and describes the resource context over time. For example, the production of one widget (an element) requires 8 hours of labor (a resource), 150 amp hours of electricity (another resource) and 5 tons of hardened steel (another resource). This layer may be subdivided into any number of sub-layers along user specified dimensions such as lexicon (what resources are called), resources already delivered, resources with delivery commitments and forecast resource requirements.

Layer 3: A layer that defines and describes the environment context over time. This layer may define and describe the entities in the social (1000), natural (2000) and/or physical environment (3000) that impact entity function and/or function measure performance. For example, the percentage of on-time shipments from supplier Z is 74% percentage of on-time shipments and from supplier A is 91%. This layer may be subdivided into any number of sub-layers along user specified dimensions.

Layer 4: A layer that defines and describes the transaction context (also referred to as tactical/administrative context) over time. For example, Company A may owe Company B $30,000 for prior sales. Company B has made a commitment to ship 100 widgets to Company A by next Tuesday and will need to start production by Friday. This layer may be subdivided into any number of sub-layers along user specified dimensions such as historical transactions, committed transactions, forecast transactions, historical events, forecast events and combinations thereof.

Layer 5: A layer that defines and describes the resilience context over time for the subject entity and for components of resilient context in an extended subject entity (950). For example, Company A is also a key supplier for the new product line. When the last hurricane hit it took Company A 4 weeks to resume shipment of the required daily part volume. This layer may be subdivided into any number of sub-layers along user specified dimensions and generally comprises a model of recovery time.

Layer 6: A layer that defines and describes the measure context over time. For example, if the price per widget is $100 and the cost of manufacturing widgets is $80, Company B can make $20 profit per unit (for most businesses this would be a short term profit measure for the value creation function). Also, Company A is one of Company B's most valuable customers and Company A is a valuable supplier to the international division (value based measures). This layer may be subdivided into any number of sub-layers along user specified dimensions. For example, the instant, five year and lifetime impact of certain medical treatments may be of interest. In this instance, three separate measurement layers could be created to provide the desired resilient context. The risks associated with each measure can be integrated within each measurement layer or they can be stored in separate layers. For example, value measures for organizations integrate the risk and the return associated with measure performance. Measures associated with other entities can be included in this layer. This capability enables the use of the difference between the subject entity (22) measure and the measures of other entities as measures;

Layer 7: A layer that defines the relationship of one or more of the first six layers of entity resilient context to one or more reference systems over time. For example, location information, such as Global Positioning System (GPS) data, can be used as the reference system for most entities. Pre-defined spatial reference coordinates available for use in the system of the present embodiment include the major organs in a human body, each of the continents, the oceans and the earth. Virtual reference coordinate systems can also be used to relate each entity to other entities. For example, a virtual coordinate system could be a network such as the Internet, an intranet, a local area network, a wi-fi network, a wimax network and/or social network. This layer may also be subdivided into any number of sub-layers along user specified dimensions and would identify system or application resilient context if appropriate.

Layer 8: A layer that defines and describes the lexicon of the subject entity (22)—this layer may be broken into sub-layers to define the lexicon associated with each of the previous resilient context layers.

Different combinations of resilient context layers from different subjects and/or entities are relevant to different analyses and decisions. The layers may be combined for ease of use, to facilitate processing and/or as entity requirements dictate. Resilient context frames are defined by one or more entity function and/or measures, and the resilient context layers impact the one or more entity function and/or measures.

The following are terms used herein in describing the Entity Resilience System (30) and applications thereof:
1. 3D printing—also referred to as additive manufacturing is a process of making three dimensional solid objects from a digital file. 3D printing is achieved using additive processes, where an object is created by laying down successive layers of material (e.g., plastic, skin, ink, etc.) with a printer.
2. Action—acquisition, consumption, destruction, production or transfer of resources, elements and/or factors at a defined point in space time—examples: blood cells transfer oxygen to muscle cells and an assembly line builds a product. Actions are a subset of events and are generally completed by a process.
3. Agent—subset of elements that can participate in an action. Six distinct kinds of agents are recognized—initiator, negotiator, closer, catalyst, regulator and messenger. A single agent may perform several agent functions—examples: customers, suppliers and salespeople.
4. Article—an instance of media.
5. Asset—subset of elements that support actions and are usually not transferred to other entities and/or consumed (e.g., automobile, lathe and oven).
6. Bot—independent components of the application software that complete specific tasks, note: also referred to as intelligent agents.
7. Characteristic—numerical or qualitative indication of entity status—examples: temperature, color, shape, distance, weight, and cholesterol level (descriptive data are the typical source of data about characteristics) and the acceptable range for these characteristics (also referred to as a subset of constraints). Characteristic data can be input as either binaries (1 for presence, 0 for absence) or as normalized values (e.g., if weight ranges between 0 and 300 pounds, then a subject entity that weights 150 pounds would have an input value of 0.5 for a weight characteristic).
8. Commitment—an obligation to complete a transaction in the future—example: contract for future sale of products and debt.
9. Competitor—subset of factors, an entity that seeks to complete the same actions as the subject, competes for elements, competes for resources or some combination thereof.
10. Competitor risk—risks that are a result of actions by an entity that competes for resources, elements, actions or some combination thereof.
11. Component of resilient context (also referred to as component of context)—factors (925), resources (926), elements (927) and/or items that make a contribution to one or more subject entity measures.
12. Composite factors (also referred to as composite variables) for a factor or factor combination are mathematical combinations of factor variables and/or factor performance indicators, logical combinations of factor variables and/or factor performance indicators and combinations thereof.
13. Composite variables for a resilient context element or element combination are mathematical combinations of item variables and/or indicators, logical combinations of item variables and/or indicators and combinations thereof.
14. Configure—to put together or arrange the parts of an offering in a specific way or for a specific purpose.
15. Contextbase—a database that organizes data and information by resilient context layer for one or more subject entities (22).
16. Contingent liability—an event risk where the impact of an event occurrence is known, can be estimated, or can be quantified
17. Contribution—the amount of variance in a measure model explained by each component of context, usually expressed as a percentage. In one embodiment the contribution is determined using component analysis.
18. Critical risk—extreme risks that can terminate a subject entity.
19. Current—a model or measure is said to be current if it was created before the end of the maximum time period before the current time specified in the system settings.
20. Data—anything that is recorded—includes transaction data, descriptive data, content, information and knowledge.
21. Deliver—to cause transfer of an offering to a subject entity.
22. Element—also referred to as a resilient context element, context element and/or as an element of context. Elements are entities owned or controlled by the subject entity (22) that participate in and/or support one or more subject entity (22) actions and/or functions without normally being consumed by the action—examples: hammock, heart, and house.
23. Element combination—two or more elements that share performance drivers to the extent that they can be analyzed as a single element.
24. Element variables or element data—the item variables, indicators and composite variables for a specific resilient context element or sub-context element.
25. Entity—an entity having a distinct and independent existence, one or more functions, and one or more characteristics.
26. Event risk is a subset of total risk. Event risk is the risk of reduced or impaired performance caused by the occurrence of an event. Event risk can be quantified by combining a forecast of event frequency with a forecast of event impact on subject entity (22) components of resilient context and the entity itself.
27. External Services (9) are services available from systems controlled by a third party. The external services may communicate with the systems described herein via a network (wired or wireless) connection. Examples of external services include search engine services, mapping services, rating services (e.g., Zagat's, Yelp, etc.), weather services, and services provided at a particular location or site (projection services, presence detection services, voice transcription services, traffic status reports, tour guide information, etc.).
28. Extreme risk—level of risk identified by extreme value bots.
29. Factor—also referred to as a resilient context factor. Factors are entities not owned or controlled by the subject entity (22) that have an impact on subject entity (22) performance—examples: commodity markets, hurricanes.
30. Factor performance indicators (also referred to as indicators) are data derived from factor related data.
31. Factor variables are the transaction data and descriptive data associated with resilient context factors.
32. Feature—a distinct element, factor or resource that can be added to or removed from the resilient context of a subject entity.
33. Functions are operations that impact the resilient context or an entity. Functions may relate to the creation, production, growth, improvement, destruction, diminution and/or maintenance of a component of resilient context and/or one or more entities. Examples: maintaining body temperature at 98.6 degrees Fahrenheit, destroying cancer cells, improving muscle tone and producing insulin.
34. Indicators (also referred to as item performance indicators and/or factor performance indicators) are data derived from data related to an item or a factor.
35. Information—data with resilient context of unknown completeness.
36. Item—an item is an instance within an element, resource or factor. For example, an individual salesman would be an “item” within the sales department element (or entity). In a similar fashion a gene would be an item within a module entity. While there are generally a plurality of items within an element, resource or factor, it is possible to have only one item within an element, resource or factor.
37. Item variables are the transaction data and descriptive data associated with an item or related group of items.
38. Keyword—a word or combination of words that will trigger the delivery of one or more advertisements, offers and/or processes to a subject entity when it appears in an article, a search and/or a predictive search (also referred to as Resilient Context Scout).
39. Knowledge—all eight types of layers for a resilient context are defined and complete for all entity functions.
40. Layer—software and/or information that gives an application, system, service, device or layer the ability to interact with another layer, device, system, service, application or set of information at a general or abstract level rather than at a detailed level.
41. Measure—quantitative indication of one or more subject entity (22) functions and/or missions—examples: cash flow, survival rate, bacteria destruction percentage, shear strength, torque, cholesterol level, and pH maintained in a range between 6.5 and 7.5.
42. Metabolome—The metabolome represents the collection of all metabolites in a biological cell, tissue, organ or organism, which are the end products of cellular processes.
43. Microbiome—one or more microbial communities that inhabit a particular organism, for example the human microbiome includes communities located in or on nasal passages, oral cavities, skin, the gastrointestinal tract and the urogenital tract, community members include bacteria and fungi.
44. Mission—a mission is an act or result associated with an entity, such as what an entity intends to do or achieve (e.g., a goal). Functions support the completion of an entity mission. An example of a default mission of a human entity is to maintain health.
45. Module—a collection of genes which share a common pattern of expression in a common set of experimental conditions.
46. Motif—a nucleotide or amino-acid sequence pattern that is widespread and has, or is conjectured to have, a biological significance. For proteins, a sequence motif is distinguished from a structural motif, a motif formed by the three dimensional arrangement of amino acids, which may not be adjacent.
47. Negative event—an event that reduces entity performance with respect to one or more function measures (also referred to as realized risk).
48. Next-gen sequencing—high-throughput sequencing methods that parallelize the sequencing process, producing thousands or millions of sequences at once, these methods include the biome representational in silico karyotyping (BRISK) method.
49. Normal range—average, plus or minus two deviations,
50. Offer—provide specific terms and conditions for completing a sale.
51. Offering—something of value made available to an entity for acquisition via an offer.
52. Performance—a measurement of mission measure and function measure levels (e.g., increases in mission measure levels are equated with increases in performance).
53. Priority—relative importance assigned to actions and/or measures.
54. Process—combination of elements, resources, factors and/or events that are used to produce an action—examples: close a sale, build a house, regulate cholesterol and provide a treatment.
55. Process map (also referred to as a protocol)—A process map characterizes the expected sequence and timing of events, commitments and actions for a medication delivery, treatment delivery or a procedure.
56. Production—a process that causes the existence of an offering.
57. Project—action or series of actions that produces one or more lasting changes. Change can include: changing a characteristic, changing a constraint, producing one or more new components of resilient context, and changing one or more components of resilient context or some combination thereof. Said changes impact entity function performance/mission.
58. Proteome—the proteome is the entire set of proteins expressed by a genome. More specifically, it is the set of expressed proteins in a given type of cell or organism at a given time under defined conditions.
59. Real options are defined as options the entity may have to make a change in its behavior/performance at some future date—these can include the introduction of new elements or resources, the ability to move processes to new locations, etc. Real options are generally supported by the elements and resources of an entity.
60. Reference Enterotypes—enterotypes are identifiable variations in the levels of different networks of bacteria that are present in a microbiome: There are currently three known human enterotypes called: Bacteroides (enterotype 1), Prevotella (enterotype 2) and Ruminococcus (enterotype 3).
61. Reference Sequence—is a nucleic acid sequence that is a representative example of an entities genes or the genes in a gene module (see module definition above), reference modules may also include motifs.
62. Requirement—minimum or maximum levels for one or more elements, element characteristics, actions, events, factors or resources.
63. Resilience—the capacity of an entity to survive, adapt, and/or grow in the face of negative events.
64. Resilience Indicator—measures that are a function of the status of an entity and/or the response of an entity to negative event. Resilience indicators are used as inputs to models of resilience measures.
65. Resilience Measure—A resilience measure is determined the amount of time required to return to a level of measure performance or output that is within some percentage of the average level that was being experienced by the subject entity or component of context before a negative event or by the magnitude of the negative event that is required to decrease measure performance or output by more than a defined percentage. These resilience measures allow for a scale and/or a classification of resilience measures. For example a magnitude 5.1 earthquake decreases measure performance by 10%, a magnitude 6.2 earthquake decreases measure performance 25% and a magnitude 7.2 earthquake decreases measure performance 50%. Another example would be it takes 3 days to return to 50% of average measure performance after a magnitude 7.5 earthquake, it takes 1 day to return to 75% of average measure performance after a magnitude 6.5 earthquake and it takes 4 hours to return to 90% of average measure performance after a 5.2 magnitude earthquake. The resilience measure used for analysis is selected in the system settings table (162) and the models that are built for resilience vary by node depth (e.g., at node depth 1, the resilience of each system is modeled along with function measure resilience. at node depth 2, the resilience of each organ and each system is modeled along with function measure resilience, etc.).
66. Resilient Context—defines and describes the relationship of a subject entity with its mission measure performance and resilience. Embodiments are shown in FIG. 7A and FIG. 7B. The resilient context may include but is not limited to the data, information and knowledge that defines and describes up to eight resilient context layers. A resilient context includes a resilience index and/or a predictive model of subject entity resilience for one or more resilience measures.
67. Resilient Context frames—a resilient context that includes information relevant to health and function measure performance for a defined combination of resilient context layers, subject entity (22) and subject entity (22) function measures.
68. Resilient Frontier—a maximum mission measure level that can be expected for a given level of risk after implementing one or more programs to improve resilience.
69. Resource—entities that are routinely transferred to other entities and/or consumed. They may be owned or controlled by the subject entity (22) (e.g., time, gasoline) or they may be independent of the subject entity (22) (e.g., air, water).
70. Risk—variability or events that reduce or degrade subject entity (22) function measure performance or function measure output.
71. Service—a set of one or more activities.
72. Services are self-contained, self-describing, modular pieces of software that can be published, located, queried and/or invoked across a World Wide Web (33), network and/or a grid. In one embodiment all services are SOAP compliant. Bots and agents can be functional equivalents to services. In one embodiment all applications are services. However, the system of the present embodiment can function using: bots (or agents), client server architecture, and integrated software application architecture and/or combinations thereof.
73. Sub-element—a subset of all items in an element that share similar characteristics.
74. Subject entity—an entity that is the subject of a resilience context analysis. An example of a subject entity is a physical entity such as a person. Other examples are shown in FIG. 7A and FIG. 7B.
75. Subresource—a subset of a specific resource group that shares similar characteristics.
76. Surprise—an event that increases entity performance with respect to one or more function measures.
77. Sustainability: a measure of its expected lifespan, it is defined by the time period when a function measure performance is kept above a certain level.
78. The efficient frontier—the curve defined by the maximum function and/or function measure performance an entity can expect for a given level of total risk for a given scenario. The normal scenario continues the actual trend over the last two years, the extreme scenario is developed using algorithms that identify extreme values and the worst case scenario is identified by letting a genetic algorithm evolve to the most negative scenario. and
79. Total risk is the sum of all variability risks and event risks for a subject.
80. Transaction—events or actions, typically involving the transfer of a resource to acquire an element or different resource. Transactions generally reflect events and/or actions for one or more entities over time (transaction data are generally the source).
81. Uncertainty measures the amount of subject entity (22) function measure performance that cannot be explained by the components of resilient context and their associated risk that have been identified by the system of the present embodiment. Sources of uncertainty include model error and data error.
82. User—the user is an entity that may or may not be the subject entity (22).
83. Variability risk—is a subset of total risk. It is the risk of reduced or impaired performance caused by variability in one or more components of resilient context. Variability risk is quantified using statistical measures like standard deviation. The covariance and dependencies between different variability risks are also determined because simulations use quantified information regarding the inter-relationship between the different risks to perform effectively. and
84. Virome—The viruses that inhabit a particular organism such as the subject entity (22).

Eight types of resilient context layers and exemplary sources for the data and information are described below, with reference to the terms provided above.

Element Context Layer: The element context layer (also referred to as element layer) identifies and describes the entities owned or controlled by the subject entity (22) that have an impact on one or more subject entity (22) functions and/or on subject entity function measure performance by time period. The element description includes the identification of any sub-elements. Elements are initially identified by the subject entity (22) hierarchy (elements associated with lower levels of a hierarchy are automatically included) whereas transaction data identifies others as do analysis and user input. These elements may be identified by item or sub-element. The sources of data can include devices (3), narrow system databases (5), partner narrow system databases (6), external databases (7), the World Wide Web (33), external services (9), XML compliant applications, the Resilient Context Input Service (601) and combinations thereof.

Resource Context Layer: The resource context layer (also referred to as resource layer) identifies and describes the resources that have an impact on subject entity (22) function and/or on subject entity function measure performance by time period. Resources may be owned or controlled by the subject entity (22) (e.g., gasoline, money) or they may be independent of the subject entity (22) (e.g., air, water). The resource description includes the identification of any sub-resources. The sources of data can include narrow system databases (5), partner narrow system databases (6), external databases (7), the World Wide Web (33), external services (9), XML compliant applications, the Resilient Context Input Service (601) and combinations thereof.

Environment Context Layer: The environment context layer (also referred to as environment layer) identifies and describes the entities and events in the social, natural and/or physical environment that are not owned or controlled by the subject entity that have an impact subject entity (22) function and/or on subject entity function measure performance by time period. The sources of data can include devices (3), narrow system databases (5), partner narrow system databases (6), external databases (7), the World Wide Web (33) and external services (9), XML compliant applications, the Resilient Context Input Service (601) and combinations thereof.

Transaction Context Layer: The transaction context layer (also referred to as transaction layer) identifies and describes any exchanges of resources or elements between the subject entity and any other entity. These exchanges may be completed in accordance with a process map or protocol. The sources of process maps can include simulation programs, the user (41), a subject matter expert (42), a collaborator (43), one or more narrow system databases (5), one or more partner narrow system databases (6), one or more external databases (7), the World Wide Web (33), one or more external services (9), one or more XML compliant applications, the Resilient Context Input Service (601) and combinations thereof.

Measure Context Layer: The measure context layer (also referred to as measure layer) quantifies the impact of actions, events, elements, factors and resources on each entity function measure by time period and identifies the relationship between the first three layers (element, resource and factor context) and the measure levels by time period. The impact of risks and surprises can be kept separate or integrated with other element/factor measures. The impacts are generally determined via analysis. However, the analysis can be supplemented by input from simulation programs, the user (41), a subject matter expert (42) and/or a collaborator (43), narrow system databases (5), partner narrow system databases (6), external databases (7), the World Wide Web (33), external services (9), XML compliant applications, the Resilient Context Input Service (601) and combinations thereof.

Resilience Context Layer: The resilience context layer (also referred to as resilience layer) comprises a model of the subject entity resilience (22) for a selected element and element measure. The resilience model is comprised of resilience indicators that are developed by analyzing data obtained from user input, narrow system databases (5), partner narrow system databases (6), external databases (7), the World Wide Web (33), external services (9), XML compliant applications, the Resilient Context Input Service (601) and combinations thereof. However, the analysis can be supplemented by input from: simulation programs, the user (41), a subject matter expert (42), social input and/or a collaborator (43), narrow system databases (5), partner narrow system databases (6), external databases (7), the World Wide Web (33), external services (9), XML compliant applications, the Resilient Context Input Service (601) and combinations thereof.

Reference Context Layer: The reference context layer (also referred to as reference layer) defines the relationship of the first six layers to a specified real (e.g., gps) or virtual coordinate system. These relationships can be identified by user input, input from a subject matter expert (42), a collaborator (43), narrow system databases (5), partner narrow system databases (6), external databases (7), the World Wide Web (33), external services (9), XML compliant applications, the Resilient Context Input Service (601), analysis and combinations thereof. and

Lexical Context Layer: The lexical context layer (also referred to as lexical layer) defines the terminology used to define and describe the components of resilient context in the other seven layers. These lexicon can be identified by user input, input from a subject matter expert (42) and/or a collaborator (43), narrow system databases (5), partner narrow system databases (6), external databases (7), the World Wide Web (33), external services (9), XML compliant applications, the Resilient Context Input Service (601), analysis and combinations thereof.

A combination of up to eight of the resilient context layers defines a resilient context for subject entity function measure performance for each node depth. The more precise definition of resilient context can be used to define what it means to be knowledgeable. Our revised definition would state that an individual that is knowledgeable about the subject entity (22) has information from all eight resilient context layers for one or more subject entity missions. This level of knowledge is important because, once the resilient context is defined and modeled; any negative events (e.g., an infection or a natural disaster) can be managed effectively. The knowledgeable individual would be able to use the information from the eight resilient context layers to identify the range of contexts where models of subject entity (22) function performance are applicable; and accurately predict subject entity (22) recovery times in response to events and/or actions in contexts where the resilient context is applicable.

The accuracy of the prediction created using the eight types of resilient context layers reflects the level of knowledge about the subject entity (22). For simplicity, the R squared (R²) statistic can be used as the measure of knowledge level. R² is the fraction of the total variance that is explained by the model—other statistics can be used to provide indications of the entity model accuracy including entropy measures. The gap between the fraction of performance explained by the model and 100% is caused by uncertainty, errors in the model and errors in the data. Table 7 illustrates the use of the information from seven of the eight layers in analyzing a sample resilient context.

TABLE 7

| | |
|---|---|
| 1. | Mission: patient health, financial break even |
| 2. | Environment: malpractice insurance is increasingly costly |
| 3. | Measure: survival rate is 99% for procedure A and 98% for procedure |
| | B; treatment in first week improves 5 year survival 18%, 5 year |
| | recurrence rate is 7% higher for procedure A |
| 4. | Resilience: 99% of patients return to work 8 to 14 days after procedure |
| | A and 6 to 10 days after procedure B; |
| 5. | Resource: operating room A time available for both procedures |
| 6. | Transaction: subject entity (22) should be treated next week, his |
| | insurance will cover operation |
| 7. | Element: operating room, operating room equipment, Dr. X and his |
| | team |

Some analytical applications are limited to optimizing the instant (short-term) impact given the elements, resources and the transaction status. Because these systems generally ignore uncertainty and the impact, reference, environment, resilience and long term measure portions of a resilient context, the recommendations they make are often at odds with common sense decisions made by line managers that have a resilient context for evaluating the same data. This deficiency is one reason some have noted that “there is no intelligence in business intelligence applications”. One reason some existing systems take this approach is that the information that defines three important parts of resilient context (relationship, environment and long term measure impact) are not readily available and must generally be derived. A related shortcoming of some of these systems is that they fail to identify the resilient context or contexts where the results of their analyses are valid. The system of the present embodiment supports the development and storage of all eight types of resilient context layers in order to create a Resilient Contextbase (50).

The Resilient Contextbase (50) also enables the development of analytical reports including a sustainability report and a controllable performance report. As shown qualitatively in Table 8, the expected subject entity (22) sustainability is a function of subject entity resilience and the expected events that will be experienced by the subject entity (22) given its resilient context.

TABLE 8

| | Low Resilience | High Resilience |
|---|---|---|
| Many negative events | Low sustainability | Moderate sustainability |
| Few negative events | Moderate sustainability | High sustainability |

As detailed below, the expected sustainability of an entity is determined by a multi-period simulation that relies on the resilient context that contains both the subject entity measure model(s) and the subject entity resilience model under one or more scenarios. Subject entity resilience is modeled using a plurality of characteristics that include: surplus capacity, effective redundancy and component independence as detailed below.

Resilient Context elements and resilient context factors are influenced to varying degrees by the actions of the subject entity (22). The controllable performance report identifies the relative contribution of the different resilient context elements, resources and/or factors to the current level of entity performance. It then puts the current level of performance in resilient context by comparing the current level of performance with the performance that would be expected if some or all of the elements, resources and/or factors were all at the mid-point of their normal range—the choice of which elements, resources and/or factors to modify is a function of the control exercised by the subject. Both of these reports are pre-defined for display using the Resilient Context Review Service (607) described below.

The Resilient Context Review Service (607) and the other services in the Resilient Context Suite (625) use resilient context frames and sub-context frames to support the analysis, forecast, review and/or optimization of entity resilience. Resilient Context frames and sub-context frames are created from the information provided by the Entity Resilience System (30). The ID to frame table (165) identifies the resilient context frame(s) and/or sub-context frame(s) that will be used by each user (41), subject matter expert (42), and/or collaborator (43). This information is used to determine which portion of the Resilient Contextbase (50) will be made available to the devices (3) and narrow systems (4) that support the user (41), subject matter expert (42), and/or collaborator (43) via the Resilient Context API (application program interface). As detailed later, the system of the present embodiment can also use other methods to provide the required resilient context information.

Resilient Context frames can be defined by the entity function and/or measures and the resilient context layers associated with the entity function and/or measures. The resilient context frame provides the data, information and knowledge that quantify the impact of actions, constraints, elements, events, factors, preferences, processes, projects, risks and resources on entity performance. Sub-context frames contain information relevant to a subset of one or more function measure/layer combinations. For example, a sub-context frame could include the portion of each of the resilient context layers that was related to an entity process. Because a process can be defined by a combination of elements, events, factors and resources that produce an action, the information from each layer that was associated with the elements, events, factors, resources and actions that define the process would be included in the sub-context frame for that process. This sub-context frame would provide all the information needed to understand process performance and the impact of events, actions, element changes, resource changes and factor changes on process performance. Resilient Context frames and sub-context frames provide the data, information and knowledge that quantify the impact of actions, constraints, elements, events, factors, preferences, processes, projects, risks and resources on entity performance and resilience. The remainder of the specification may refer to resilient context frames and sub-context frames. However, it should be understood that resilient context frames and subcontext frames comprise resilient context frames and resilient sub-context frames.

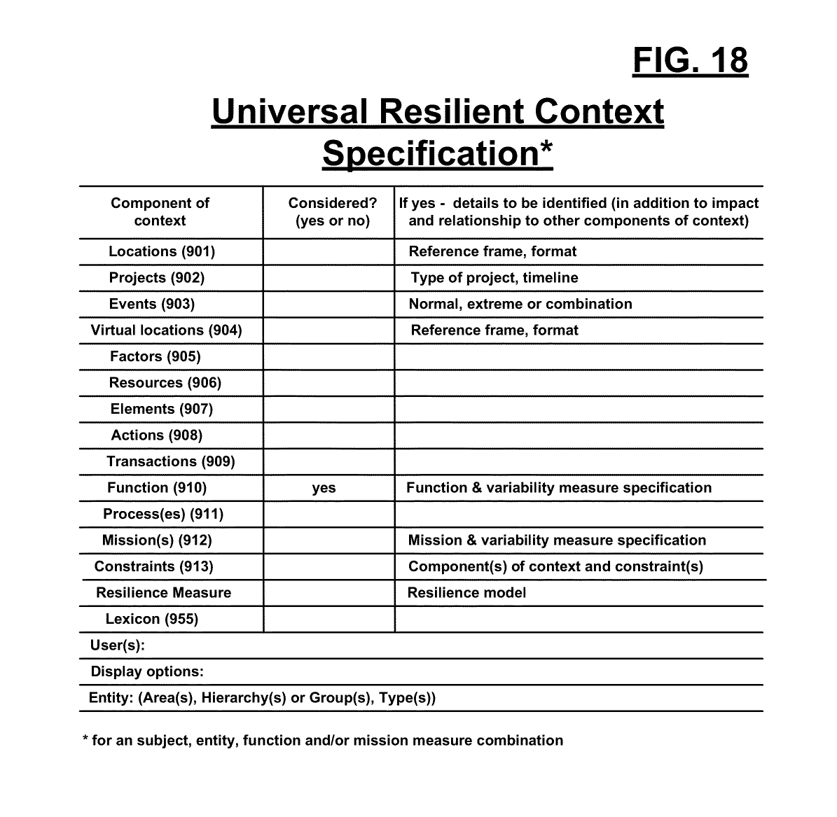

The services in the Resilient Context Suite (625) are “context aware” with resilient context quotients equal to 300 and have the ability to process data from the Entity Resilience System (30) and the Resilient Contextbase (50). Another feature of the services in the Resilient Context Suite (625) is that they can review resilient entity resilient context from prior time periods to generate reports that highlight changes over time and display the range of contexts under which the results they produce are valid. The range of contexts where results are valid will be hereinafter be referred to as the valid resilient context space. The services in the Resilient Context Suite (625) also support the development of customized applications or services. They do this by providing ready access to the internal logic of the service while at the same time protecting this logic from change and using the universal resilient context specification (see FIG. 18) to define standardized Application Program Interfaces (API) for all Resilient Context Services—these API allow the specification of the different resilient context layers using text information, numerical information and/or graphical representations of subject entity (22) resilient context in a knowledge graph format similar to that shown in FIG. 7A and FIG. 7B. The first features allow users (41), partners and external services to get information tailored to a specific resilient context while preserving the ability to upgrade the services at a later date in an automated fashion. The second feature allows others to incorporate the Resilient Context Services into other applications and/or services. It is worth noting that this awareness of the resilient context is also used to support a true natural language interface (714)—one that understands the meaning of the identified words—to each of the services in the Suite (625). It should be also noted that each of the services in the Suite (625) supports the use of a reference coordinate system for displaying the results of their processing when one is specified for use by the user (41). The software for each service in the Suite (625) resides in an intelligent agent with the resilient context frame being provided by the software in the Entity Resilience System (30) which is also comprised of bots (also referred to as intelligent agents or components). Other features of the services in the Resilient Context Suite (625) are briefly described below:

Resilient Context Analysis Service (602)—analyzes the impact of user (41) specified changes on the subject entity (22) for a given resilient context frame or sub-context frame by mapping the proposed change to the appropriate resilient context layer(s) in accordance with the schema or ontology and then evaluating the impact of said change on the function and/or measures. Resilient Context frame information may be supplemented by simulations and information from subject matter experts (42) as appropriate. This service can also be used to analyze the impact on changes on any “view” of the entity that has been defined and pre-programmed for review. For example, accounting profit using three different standards (GAAP, IFRS and cash) or capital adequacy can be analyzed using the same rules defined for the Resilient Context Review Service (607) to convert the resilient context frame analysis to the required reporting format.

Resilient Context Auditing Service (624)—re-processes all transactions and compares the resulting values with the information in one or more reports presented by management. The Resilient Context Auditing Service then combines this information with the information stored in the Resilient Contextbase (50) to complete an automated audit of all the numbers in a report—including reserve estimates. After the various calculations are completed, the system of the present embodiment produces a discrepancy report where the reported values in a report is compared to the value computed using the method and system detailed above.

Resilient Context Benefit Plan Analysis Service (629)—service that combines information regarding any pension or health care benefit plans from a benefits administration system or other source with the expected sustainable longevity and the expected events of the entities covered by the pension or health care benefit plan. The subject entity can be an individual covered by said plan or the organization offering said plan. As is well known in the art, pension benefit plans generally rely on actuarial assumptions regarding the expected longevity of covered employees and their covered relatives (e.g., spouses). Pension benefit amounts are generally based on years of service and salary history. The expected longevity of the covered employees and relatives are combined with the expected benefit amounts to estimate the liability associated with providing pension benefits by multiplying the number of years covered (expected longevity minus retirement age) by the plan benefit amounts. In a similar manner, the forecast of expenditures for health care benefit plans are generally developed by using historical medical claims data for individuals with demographics similar to those of covered employees and their relatives. The expected expenditures are compared to the benefits provided by the health care plans to its employees in order to estimate the expenditures that will be required to support the health care plan by multiplying the expected covered expenditures for each demographic category by the number of people in each category. The Resilient Context Benefit Plan Analysis Service compares the expected expenditure forecast produced using the traditional methods described above for said pension and/or health care benefit plans for the subject entity (22) with a forecast of subject entity (22) related expenses based on the expected sustainable longevity (as described above, sustainable longevity is a product of expected events and resiliency—see Table 8) in order to forecast the variance in expenditures and risk associated with providing pension and health care coverage. These estimates can be calculated using simple mathematical calculations (plan forecast—Entity Resilience System (30) forecast of subject entity (22) related expenses), the Resilient Context Forecast Service (603) or simulation. The expected sustainable longevity and the expected events of the subject entity (22) can also be combined with financial information for a hospital, nursing home, assisted care facility or health care provider such as a health maintenance organization to forecast the short and long term expenses associated with providing care for the subject entity (22) using the Resilient Context Forecast Service (603) or simulation. A relatively new benefit some companies are now providing is a wellness program for their employees. Models of health care functions can be used to identify changes that can be made to improve employee wellness. The impact of these changes on expected sustainability and events can be estimated using the sustainability and event models detailed herein. These changes can be used to estimate the impact of said wellness programs on health care and pension benefit plans. Expenditures on wellness could be optimized by completing an analysis of the tradeoffs between increased wellness expenditures, decreased health insurance expenditures and increased employee pension expenditures using the Resilient Context Optimization Service (604).

Resilient Context Bridge Service (624)—is a service that identifies the differences between two resilient context frames and an optimized mode for bringing the frames into alignment or congruence. This service can be very useful in breaking down barriers to communication and facilitating negotiations.

Resilient Context Browser (628)—supports browsing through the Resilient Contextbase (50) with a focus on one or more dimensions of the Universal Resilient Context Specification for the user (41) and/or a subject.

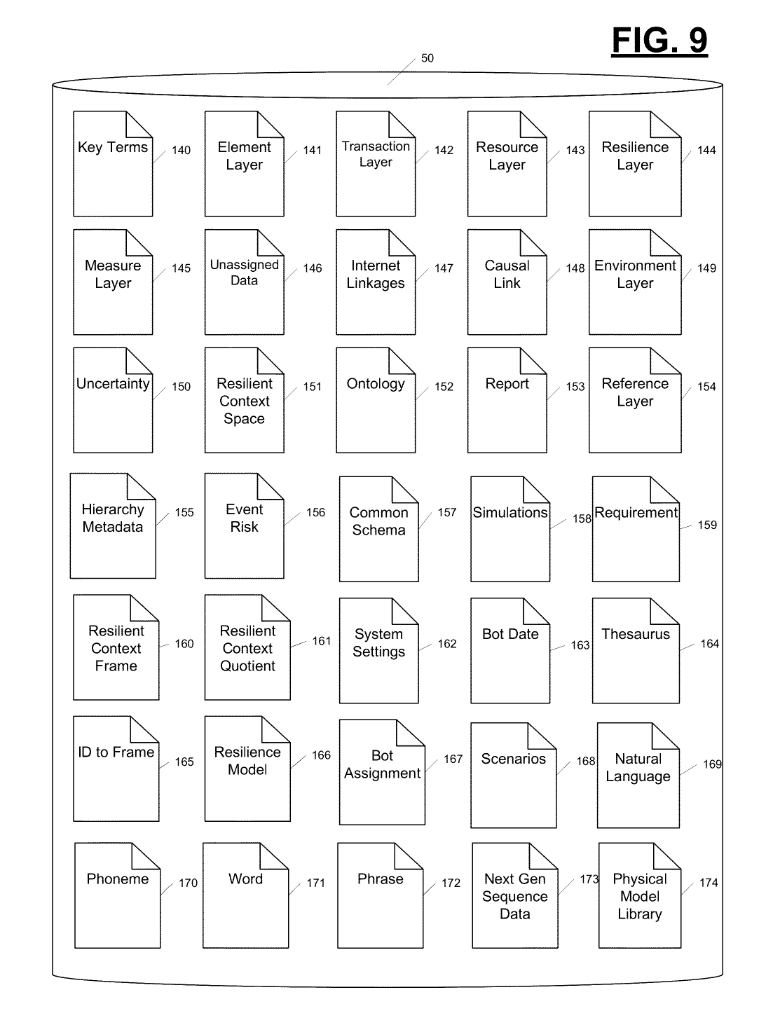

Resilient Context Capture and Collaboration Service (622)—guides one or more subject matter experts (42) and/or collaborators (43) through a series of steps in order to capture information, refine existing knowledge and/or develop plans for the future using existing knowledge using a knowledge capture window (707). The subject matter experts (42) and/or collaborators (43) can provide information and knowledge by selecting from a template of pre-defined elements, resources, events, factors, actions and entity hierarchy graphics that are developed from the common schema. The subject matter experts (42) and/or collaborators (43) also have the option of defining new elements, events, factors, actions and hierarchies. The subject matter experts (42) and/or collaborators (43) are first asked to define what type of information and knowledge will be provided. The choices will include each of the eight types of resilient context layers as well as element definitions, factor definitions, event definitions, action definition, impacts, processes, uncertainty and scenarios. On this same screen, the subject matter experts (42) and/or collaborators (43) will also be asked to decide whether basic structures or probabilistic structures will be provided in this session, if this session will require the use of a time-line and if the session will include the lower level subject matter. The selection regarding type of structures will determine what type of samples will be displayed on the next screen. If the use of a time-line is indicated, then the user will be prompted to: select a reference point—examples would include today, event occurrence, when I started, etc.; define the scale being used to separate different times—examples would include seconds, minutes, days, years, light years, etc.; and specify the number of time slices being specified in this session. The selection regarding which type of information and knowledge will be provided determines the display for the last selection made on this screen. There is a natural hierarchy to the different types of information and knowledge that can be provided by the subject matter experts (42) and/or collaborators (43). For example, measure level knowledge would be expected to include input from the element, environment, transaction and resource context layers. If the subject matter experts (42) and/or collaborators (43) agree, the service will guide the subject matter experts (42) and/or collaborators (43) to provide knowledge for each of the “lower level” knowledge areas by following the natural hierarchies. Summarizing the preceding discussion, the subject matter experts (42) and/or collaborators (43) has used the first screen to select the type of information and knowledge to be provided (measure layer, transaction layer, resource layer, environment layer, element layer, reference layer, event risk or scenario). The subject matter experts (42) and/or collaborators (43) have also chosen to provide this information in one of four formats: basic structure without timeline, basic structure with timeline, relational structure without timeline or relational structure with timeline. Finally, the subject matter experts (42) and/or collaborators (43) have indicated whether or not the session will include an extension to capture “lower level” knowledge. Each selection made by the subject matter experts (42) and/or collaborators (43) will be used to identify the combination of elements, events, actions, factors and entity hierarchy chosen for display and possible selection. This information will be displayed in a manner that is somewhat similar to the manner in which stencils are made available to Visio® users for use in the workspace. The next screen displayed by the service will depend on which combination of information, knowledge, structure and timeline selections that were made by the subject matter experts (42) and/or collaborators (43). In addition to displaying the sample graphics to the subject matter experts (42) and/or collaborators (43), this screen will also provide the subject matter experts (42) and/or collaborators (43) with the option to use graphical operations to change impacts, define new impacts, define new elements, define new factors and/or define new events. The thesaurus table (164) in the Resilient Contextbase (50) provides graphical operators for: adding an element or factor, acquiring an element, consuming an element, changing an element, factor or event risk values, adding an impact, changing the strength of an impact, identifying an event cycle, identifying a random impact, identifying commitments, identifying constraints and indicating preferences. The subject matter experts (42) and/or collaborators (43) would be expected to select the structure that most closely resembles the knowledge that is being communicated or refined and add it to the workspace being displayed. After adding it to the workspace, the subject matter experts (42) and/or collaborators (43) will then edit elements, factors, resources and events and add elements, factors, resources, events and descriptive information in order to fully describe the information or knowledge being captured from the resilient context frame represented on the screen. If relational information is being specified, then the subject matter experts (42) and/or collaborators (43) will be given the option of using graphs, numbers or letter grades to communicate the information regarding probabilities. If a timeline is being used, then the next screen displayed will be the screen for the same perspective from the next time period in the time line. The starting point for the next period knowledge capture will be the final version of the knowledge captured in the prior time period. After completing the knowledge capture for each time period for a given level, the Service (622) will guide the subject matter experts (42) and/or collaborators (43) to the “lower level” areas where the process will be repeated using samples that are appropriate to the resilient context layer or area being reviewed. At all steps in the process, the information in the Resilient Contextbase (50) and the knowledge collected during the session will be used to predict elements, resources, actions, events and impacts that are likely to be added or modified in the workspace. These “predictions” are displayed using flashing symbols in the workspace. The subject matter experts (42) and/or collaborators (43) are given with the option of turning the predictive prompting feature off. After the information and knowledge has been captured, the graphical results are converted to data base entries and stored in the appropriate tables (141, 142, 143, 144, 145, 149, 154, 156, 157, 158, 162 or 168, shown in FIG. 9) in the Resilient Contextbase (50). Data from simulation programs can also be added to the Resilient Contextbase (50) to provide similar information or knowledge. This Service (622) can also be used to verify the veracity of some new assertion by mapping the new assertion to the subject entity (22) model and quantifying any reduction in explanatory power and/or increase in uncertainty of the entity performance model. The capture and collaboration service (622) can also be used to collect “social input” for use as input to measure models and/or resilience models from entities that are not subject matter experts. This input may be weighted using the methods detailed under the Resilient Context Social Underwriting Service (639) detailed below.

Resilient Context Compliance Service (626)—service that can be run in real time, daily, weekly, monthly, quarterly or yearly for the subject entity (22). The service compares the specified requirements to the actual levels observed for account balances, risks, transactions and/or values over the specified time period and provides reports highlighting any differences between requirements and actual levels.

Resilient Context Customization Service (621)—service for analyzing and optimizing the impact of data, information, products, projects and/or services by customizing the features included in or expressed by an offering for the subject entity (22) for a given resilient context frame or sub-context frame. The resilient context frame or sub-context frame may be provided by the Resilient Context Summary Service (617). Some of the products and services that can be customized with this service include medicine, medical treatments, medical tests, software, technical support, equipment, computer hardware, devices, services, telecommunication equipment, living space, buildings, advertising, data, information and knowledge. Products that can be produced by 3D printers can also be customized if the data files used to guide the production of products with said printer contain modular features that can be selected for inclusion or deletion. Other customizations may rely on the Resilient Context Optimization Service (604) working alone or in combination with the Resilient Context Search Service (609). Resilient Context frame information may be supplemented by simulations and information from subject matter experts (42) as appropriate.

Resilient Context Exchange Service (608)—identifies desirable exchanges of resources, elements, commitments, data and information with other entities for the subject entity (22) in an automated fashion. This service calls on Resilient Context Analysis Service (602) in order to review proposed prices. In a similar manner the service calls on the Resilient Context Optimization Service (604) to determine the optimal parameters for an exchange before completing a transaction. For partners or customers that provide access to their data that are sufficient to define a shared resilient context, the exchange service can use the other services from the Resilient Context Suite (625) to analyze and optimize the exchange for the combined parties. The actual transactions are completed by the Resilient Context Input Service (601).

Resilient Context Forecast Service (603)—forecasts the value of specified variable(s). The service 603 completes a tournament of forecasts for specified variables and defaults to an overage of a combination of the three best forecasts from the tournament. Forecasts are created by using the actual history from the time periods (e.g., 15 to 24 time periods) that precede the base period established in the system settings table (162) together with different algorithms to produce different forecasts covering the base period (e.g., thirty different algorithms to produce thirty different forecasts). The thirty different algorithms used in calculating preliminary forecasts are: prior 3 period average; prior 6 period average; prior 12 period average; prior 15 period average prior 18 period average, prior 24 period average, prior period actual, prior period actual times (prior period actual/2 periods prior actual), prior period actual times (1+3 period average period-to-period trend), prior period actual times (1+6 period average period-to-period trend), prior period actual times (1+12 period average period-to-period trend), prior period one quarter ago, prior period two quarters ago, prior period one year ago (seasonal), prior period two years ago, average of (prior period one year ago+prior period one period before the period one year ago+prior period one period after one year ago), average quarter during last year that is converted to daily, weekly or monthly forecast as appropriate, average quarter during last year times (1+most recent quarter-to-quarter growth rate), average quarter during last year times (1+average quarterly growth last year) that is converted to monthly or weekly forecast as appropriate, average period last year, average period last year times (1+average period growth last year), simple weighted average, double weighting to most recent 3 periods, damped trend exponential smoothing—reduced time period, damped trend exponential smoothing, single exponential smoothing—reduced time period, single exponential smoothing, double exponential smoothing—reduced time period, double exponential smoothing, Winters exponential smoothing—reduced time period and Holt-Winters exponential smoothing. The error of the resulting forecasts is then assessed on two parameters, magnitude (e.g., currency level, price or item volume) and trend. The magnitude error is assessed by using an error measure comprised of summing the square of the differences between the base period forecast and the actual base period results for each period and dividing the result by the number of periods where: n=period number 1, 2 . . . N; N=total number of periods in the base period; Q.sub.fn=quantity forecast for period n in base period; Q.sub.an=actual quantity during period n in base period. Trend is defined as the slope of the best-fit least-squares regression of the base period forecast. Where: n=period number 1, 2 . . . N; n−1=period prior to period n; Q.sub.n=quantity forecast for period n; Q.sub.(n−1)=quantity forecast for period prior to period n; T=trend; B=constant. The error in the trend forecast is assessed using an error measure comprised of the square of the differences between the forecast trend and the actual trend where: T.sub.f=trend of base period forecast and T.sub.a=actual trend during the base period. The error of each of the 30 forecasts is assessed using the two measures and the results for each measure are then normalized. The resulting error measures are then added together to produce an overall error measure of forecast error. Given the preceding error definitions it is clear that the lower the error measure is—the higher the forecast accuracy. The results from the three algorithms that produced the closest match with the actual base period results (the three algorithms with the lowest combined error) are averaged to produce future forecasts.

Resilient Context Indexing Service (619)—service for developing composite and covering indices for data, information and knowledge in Resilient Contextbase (50) using the impact cutoff and node depth specified by the user (41) in the system settings table (162) for searching and scouting services.

Resilient Context Input Service (601)—service for recording actions and commitments into the Resilient Contextbase (50). The interface for this service is a template accessed via a browser (800) or the natural language interface (714) provided by the system of the present embodiment (30) that identifies the available element, transaction, resource and measure data for inclusion in a transaction. After the user has recorded a transaction the service saves the information regarding each action or commitment to the Resilient Contextbase (50). Other services such as Resilient Context Analysis (602), Planning (605) or Optimization (604) Services can interface with this service to generate actions, commitments and/or transactions in an automated fashion. Resilient Context Bots (650) can also be programmed to provide this functionality.

Resilient Context Journal Service (630) (also referred to as the “daily me”)—uses natural language generation to automatically develop and deliver a prioritized summary of news and information in any combination of formats covering a specified time period (hourly, daily, weekly, etc.) that is relevant to a given subject entity (22) resilient context or resilient context frame. Relevance is determined in a manner identical to that described previously for the Resilient Context Scout Service (616) except for the fact that the user (41) is free to modify the node depth, subject entity (22) definition and/or impact cutoff used for evaluating search relevance.

Resilient Context Metrics and Rules Service (611)—tracks and displays the causal performance indicators for resilient context elements, resources and factors for a given resilient context frame for a given subject entity (22) as well as the rules used for segmenting resilient context components into smaller groups for more detailed analysis. Rules and patterns can be discovered using an algorithm tournament that includes the Apriori algorithm, the sliding window algorithm; differential association rule mining, beam-search, frequent pattern growth and decision trees.

Resilient Context Optimization Service (604)—simulates the subject entity (22) performance using Monte Carlo simulation and identifies the optimal mix of actions for operating a specific resilient context frame or resilient sub-context frame for one or more defined functions/measures for one or more scenarios. The scenarios can be user specified scenarios. The optimization analysis will optionally consider the impact of one or more resilience programs on the one or more specified measures for one or more scenarios before analyses are completed. If the resilience programs are analyzed, then a return on resilience will be calculated and a forecast of the resilience indices for each event risk and for the entity will be created. The return on resilience considers both the reduction in losses caused by increased resilience as well as any reduction in expense associated with risk transfer that is caused by the improved resilience. A tournament of optimization analyses is used to select the best algorithm from the group consisting of genetic algorithms, the calculus of variations, constraint programming, game theory, mixed integer linear programming, multi-criteria maximization, linear programming, semi-definite programming, smoothing and highly optimized tolerance for each scenario and measure combination. This service can also be used to optimize Resilient Context Review Service (607) measures using the same rules defined for the Resilient Context Review Service (607) to define resilient context frames before optimization.

Resilient Context Planning Service (605)—service that is used to: establish measure priorities, establish action priorities, and establish expected performance levels (also referred to as budgets) for actions, events, elements resources and measures for the subject entity (22). These priorities and performance level expectations are saved in the corresponding layer in the Resilient Contextbase (50). For example, measure priorities are saved in the measure layer table (145). This service also supports collaborative planning when resilient context frames that include one or more partners are created (see FIG. 7B).

Resilient Context Profiling Service (615)—service for developing the best estimate of a resilient context frame from available entity related data and information. If a Resilient Context has been developed for a similar entity, then the Resilient Context Profiling Service (615) will identify: the portion of behavior that is generally explained by the level of detail in the profile, differences from the similar entity, expected ranges of behavior and sources of data that are generally used to produce a more Resilient Context before completing an analysis of the available data.

Resilient Context Review Service (607)—service for reviewing components of resilient context and measures alone or in combination. These reviews can be completed with or without the use of a reference layer. This service uses a rules engine to transform Resilient Contextbase (50) historical information into standardized reports that have been defined by different entities (e.g., IFRS (International Financial Reporting Standards) financial statements, Basel III liquidity and leverage reports, etc.). The sustainability and controllable performance reports described previously are also pre-defined for calculation and display. The rules engine produces each of these reports on demand for review and optional publication.

Resilient Context Scout Service (616)—service that works with the Resilient Context Indexing Service (619) to proactively identify data, information and/or knowledge regarding choices the subject entity (22) will be making in the near future using the time frame or time frames defined by user (41) in system settings table (162). The Resilient Context Scout (616) uses process maps, preferences and the Resilient Context Forecast Service (603) to identify the choices that it expects the subject entity (22) to make in the near future. It then uses weight of evidence/satisfaction algorithms including banburismus to determine which choices need additional data, information and/or knowledge to support an informed decision within parameters selected by the user (41) in the system settings table (162). It of course, also determines which choices are already supported by sufficient data, information and/or knowledge. The relative priority given to the data, information and/or knowledge selected by the Resilient Context Scout (616) is a function of the relevance ranking produced by one of several measures of relevance including ontology alignment measures, semantic alignment measures, cover density rankings, vector space model measurements, okapi similarity measurements, three level relevance scores and hypertext induced topic selection algorithm scores. The Resilient Context Scout Service (616) evaluates relevance by utilizing the relationships and impacts that define a resilient context to the node depth and impact cutoff specified by the user in the system settings table (162) as the basis for scoring by using the techniques outlined above. The node depth identifies the number of node connections that are used to identify components of resilient context to be considered in determining the relevance score. For example, if a single entity (as shown in FIG. 7A) was expected to need information about a resource (926) and a node depth of one had been selected, then the relevance rankings would consider the components of resilient context that are linked to resources by a single link. Using this approach data, information and/or knowledge that contains and/or is closely linked to a similar mix of resilient context components will receive a higher ranking. As shown in FIG. 7A, this would include projects (922), events (923), reference locations (924), factors (925), resources (926) and elements (927) that had an impact greater than or equal to the impact cutoff on a measure. The Resilient Context Scout Service (616) has the ability to use word sense disambiguation algorithms to clarify the terms being selected for search, normalizes the terms selected for search using the Porter Stemming algorithm or an equivalent and uses collaborative filtering to learn the combination of ranking methods that are generally preferred for identifying relevant data, information and/or knowledge given the choices being faced by the subject entity (22) for each resilient context and/or resilient context frame.

Resilient Context Search Service (609)—service for locating the most relevant data, information, services and/or knowledge for a given resilient context frame or sub-context frame in one of two modes—direct or indirect. In the direct mode, the relevant data, information and/or services are identified and presented to the user (41). In the indirect mode, candidate data, information and/or services are identified using publicly available search engine results that are re-analyzed before presentation to the user (41). This service can be combined with the Resilient Context Customization Service (621) to identify and provide customized ads and/or other information related to a given resilient context frame as relevance increases (through movement relative to a reference frame, external changes, etc.). Relevance is determined in a manner identical to that described previously for the Resilient Context Scout (616) save for the fact that the user (41) is free to modify the node depth, subject entity (22) definition and/or impact cutoff used for evaluating relevance using a wizard. Any indices associated with the revised subject entity (22) definitions would automatically be changed by the Resilient Context Index Service (619) as required to support the changed definition. The user (41) could choose to change the subject entity (22) definition for any number of reasons. For example, he or she may wish to focus on only one entity resilient context for a vertical search. Another reason for changing the definition would be to incorporate one or more contexts from other entities in a new definition. For example, an employee could choose to search for information relevant to a combination of one or more of his or her contexts (for example, his or her employee resilient context) and one or more contexts of the employer/company (for example, the resilient context of his project or division). As part of its processing, the Resilient Context Search Engine (609) identifies the relationship between the requested information and other information by using the relationships and measure impacts identified in the Resilient Contextbase (50). It uses this information to display the related data and/or information in a graphical format similar to the formats used in FIG. 7A and/or FIG. 7B. Again, the node depth cutoff is used to determine how “deep” into the graph the search is performed. The user (41) has the option of focusing on any block in a graphical summary of relevant information using the Resilient Context Browser (628), for example the user (41) could choose to retrieve information about the resources (926) that support an entity (920). The subject entity (22) may not be the user (41). If this is the case, then the user's resilient context is not considered as part of normal processing. Information obtained from the natural language interface (714) could be part of this resilient context;

Resilient Context Social Underwriting Service (639)—analyzes a resilient context frame or sub-context frame for a subject entity together with “social input” regarding the entity provided by one or more other entities. The social input may be used in order to: evaluate entity liquidity (need for cash resource vs. available cash resources under a scenario), evaluate entity creditworthiness (ability to meet commitments for cash resource delivery given projected need for cash resources and available cash resources under a scenario), evaluate entity risks (complete one of more entity simulations and identify expected drop in entity measure performance for a scenario and sources of risk that contribute to said drop) and/or complete a valuation of the entity (forecast value of one or more entity measures over time). The service can then use this information to support the: transfer of liquidity to or from said entity, transfer of risks to or from said entity, securitize one or more entity risks, underwrite entity related securities, package entity related securities into funds or portfolios with similar characteristics (e.g., resilience, risk, uncertainty equivalent, value, etc.) and/or package entity related securities into funds or portfolios with dissimilar characteristics (e.g., resilience, risk, uncertainty equivalent, value, etc.). The input from one or more other entities can take the form of providing answers to a list of questions about the entity, rating the entity on one or more numerical scales, changing a rating given to the entity on one or more scales and/or indicating if the entity is liked or disliked. The input from the users can optionally be weighted based on: past experience in forecasting whereby the input from entities providing the most accurate input in the past are weighted more heavily, the results of a risk IQ test whereby the input from entities with the highest risk IQ are weighted more heavily or a combination thereof. The user (41) is given the option of determining if social underwriting will be used and if it is used, what type of weighting should be used for entity input in the system settings table (162).

Resilient Context Summary Service (617)—develops a summary of the subject entity (22) resilient context using the Universal Resilient Context Specification (see FIG. 18) in an RDF format that contains the portion of the resilient context approved for release by the user (41) for use by other applications, services and/or entities. For example, the user (41) could send a summary of two contexts (family member and church-member) to a financial planner for use in establishing a portfolio that will help the user (41) realize his or her goals with respect to these two contexts. This Resilient Context Summary can be used by others providing goods, services and information (such as other search engines) to tailor their offerings to the portion of resilient context that has been revealed.

Resilient Context Underwriting Service (620)—analyzes a resilient context frame or sub-context frame for the subject entity (22) in order to: evaluate entity liquidity (need for cash resource vs. available cash resources under a scenario), evaluate entity creditworthiness (ability to pay bills given projected need for cash resources and available cash resources under a scenario), evaluate entity risks (complete one of more entity simulations and identify expected drop in entity performance for a scenario and sources of risk that contribute) and/or complete valuations. It can then use this information to support the: transfer of liquidity to or from said entity, transfer of risks to or from said entity, securitize one or more entity risks, underwrite entity related securities, package entity related securities into funds or portfolios with similar characteristics (e.g., resilience, risk, uncertainty equivalent, value, etc.) and/or package entity related securities into funds or portfolios with dissimilar characteristics (e.g., resilience, risk, uncertainty equivalent, value, etc.). As part of securitizing entity risks the Resilient Context Underwriting Service (620) identifies an uncertainty equivalent for the risks being underwritten. This innovative analysis combines quantified uncertainty by type with the quantified risks to give investors a more complete picture of the risk they are assuming when they buy a risk security. All of these analyses can rely on the measure layer information stored in the Resilient Contextbase (50), the sustainability reports, the controllable performance reports and any pre-defined review format. Resilient Context frame information may be supplemented by simulations and information from subject matter experts as appropriate.

The services within the Resilient Context Suite (625) can be combined in any combination in order to complete a specific task. For example, the Resilient Context Review Service (607), the Resilient Context Forecast Service (603) and the Resilient Context Planning Service (605) can be joined together to process a series of calculations. The Resilient Context Analysis Service (602) and the Resilient Context Optimization Service (604) can be joined together to support performance improvement activities. In a similar fashion the Resilient Context Optimization Service (604) and the Resilient Context Capture and Collaboration Service (622) can be combined to support knowledge transfer and simulation based training. The services in the Resilient Context Suite (625) will hereinafter be referred to as the standard services or the services in the Suite (625).

The Entity Resilience System (30) utilizes a software and system architecture for developing the resilient entity resilient context used to support resilient context systems and services. Narrow systems (4) generally try to develop and use a picture of how part of an entity is performing (e.g., supply chain, heart functionality, etc.). The user (41) is then left with an enormous effort to integrate these different pictures—often developed from different perspectives—to form a complete picture of entity performance. By way of contrast, the Entity Resilience System (30) develops complete pictures of entity performance for every function using a common format (e.g., see FIG. 7A and/or FIG. 7B) before combining these pictures to define the resilient context and a Resilient Contextbase (50) for the subject. The detailed information from the resilient context is then divided and recombined in a resilient context frame or sub-context frame that is used by the standard services in any variety of combinations for analysis and performance management.

The Resilient Contextbase (50) and resilient entity contexts are continually updated by the software in the Entity Resilience System (30). As a result, changes are automatically discovered and incorporated into the processing and analysis completed by the Entity Resilience System (30). Developing the complete picture first, instead of trying to put it together from dozens of different pieces can allow the system of the present embodiment to reduce IT infrastructure complexity by orders of magnitude while dramatically increasing the ability to analyze and manage subject entity (22) performance. The ability to use the same software services to analyze, manage, review and optimize performance of entities at different levels within a domain hierarchy and entities from a wide variety of different domains further magnifies the benefits associated with the simplification enabled by the novel software and system architecture of the present embodiment.

The Entity Resilience System (30) can provide several other important features, including: the system learns from the data which means that it supports the management of new aspects of entity performance as they become important without having to develop a new system; the user is free to specify any combination of functions and measures for analysis; and support for the automated development and use of bots and other independent software applications (such as services) that can be used to, among other things, initiate actions, complete actions, respond to events, seek information from other entities and provide information to other entities in an automated fashion.

The services in the Resilient Context Suite (625) work together with the Entity Resilience System (30) to provide knowledge based support to anyone trying to analyze, manage and/or optimize actions, processes and outcomes for any subject entity (22) that experiences a negative event. The Resilient Contextbase (50) supports the services in the Resilient Context Suite (625) as described above. The Resilient Contextbase (50) can provide several important benefits. First, by directly supporting entity performance, the system of the present embodiment guarantees that the Resilient Contextbase (50) will provide a tangible benefit to the entity. Second, the measure focus allows the system to partition the search space into two areas with different levels of processing. Data and information that is known to be relevant to the defined functions and/or measures as well as data that are not thought to be relevant. The system does not ignore data that is not known to be relevant; however, it is processed less intensely. This information can also be used to identify data for archiving or disposal. The processing completed in Resilient Contextbase (50) development defines and maintains the relevant schema or ontology for the entity. This schema or ontology can be flexibly matched with other ontologies in order to interact with other entities that have organized their information using a different ontology. This functionality also enables the automated extraction and integration of data from the semantic web. Defining the resilient context allows every piece of data that is generated to be placed “in resilient context” when it is first created. Traditional systems generally treat every piece of data in an undifferentiated fashion. As a result, separate efforts are often required to find the data, define a resilient context and then place the data in resilient context. The focus on primary subject entity (22) mission also ensures the relevance of the Resilient Contextbase (50).

Some of the important features of the subject entity (22) centric approach are summarized in Table 9.

TABLE 9

| Characteristic | Entity Resilience System (30) |
|---|---|
| Tangible benefit | Built-in |
| Computation/Search Space | Partitioned |
| Ontology development and maintenance | Automated |
| Ability to analyze new element, resource or factor | Automatic-learns from data |
| Measures in alignment | Automatic |
| Data stored in resilient context | Automatic |
| Service longevity | Equal to longevity of definable measure(s) |

To facilitate its use as a tool for improving performance, the Entity Resilience System (30) produces reports in formats that are graphical and highly intuitive. By combining this capability with the previously described capabilities (developing resilient contexts, flexibly defining robust performance measures, optimizing performance, reducing IT complexity and facilitating collaboration) the Entity Resilience System (30) gives individuals, groups and clinicians the tools they need to model, manage and improve the performance of any subject entity (22).

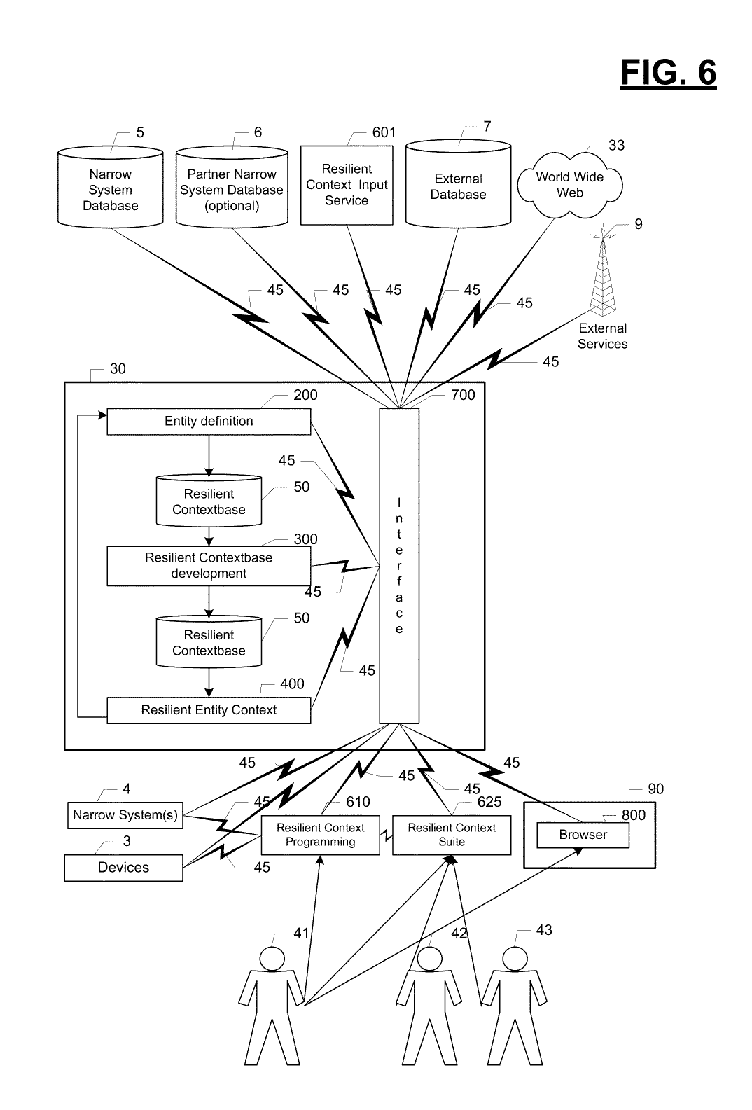

FIG. 6 provides an overview of the processing completed by the Entity Resilience System (30). In accordance with the present embodiment, an automated system and method for developing a Resilient Contextbase (50) that supports the development of an Entity Resilience System (30) is provided. In one preferred embodiment the Resilient Contextbase (50) contains a plurality of resilient context layers. Processing starts when the data preparation portion of the application software (200) extracts data, information or knowledge from at least one source such as a narrow system database (5); an external database (7); a World Wide Web (33) or an external service (9). External services may also include data feeds or streaming data. Data, information and knowledge are also optionally obtained from one or more partner narrow system databases (6) via a network (45). The connection to the network (45) can be via a wired connection, a wireless connection or a combination thereof. It is to be understood that the World Wide Web (33) also includes the semantic web that is being developed. Data may also be obtained from a Resilient Context Input Service (601) or other applications that can provide XML output.

After data are prepared, subject entity (22) functions are defined and subject entity (22) measures are identified. Models of subject entity (22) measure performance, mission performance and resilience are then developed and stored in the Resilient Contextbase (50). The Resilient Contextbase (50) is then used to support the Resilient Context Suite (625) of services in the third stage of processing. The processing completed by the Entity Resilience System (30) may be influenced by a user (41) through interaction with a user-interface portion of the application software (700) that mediates the display, transmission and receipt of all information to and from the Resilient Context Input Service (601) or browser software (800) in an access device (90) such as a mobile phone, personal digital assistant, tablet or personal computer where data are entered by the user (41). The user (41) can also use a natural language interface (714) provided by the Entity Resilience System (30).

While only one database of each type (5, 6 and 7) is shown in FIG. 6, it is to be understood that the Entity Resilience System (30) can process information from any combination of the narrow systems (4) listed in Tables 1, 2, 3 and/or 4 as well as the devices (3) listed in Table 5 for each entity being supported. In one embodiment, all functioning narrow systems (4) associated with each entity will provide data access to the Entity Resilience System (30) via the network (45). It should also be understood that it is possible to complete a bulk extraction of data from each database (5, 6 and 7), the World Wide Web (33) and external service (9) via the network (45) using peer to peer networking and data extraction applications. The data could also be stored in a database, datamart, data warehouse. Other options for data storage include a cluster (accessed via GPFS), a virtual repository or a storage area network where the analysis bots could operate on the aggregated data.

The operation of the system of the present embodiment is determined by the options the user (41) specifies and stores in the Resilient Contextbase (50). As shown in FIG. 9, the Resilient Contextbase (50) contains tables for storing data including: a key terms table (140), a element layer table (141), a transaction layer table (142), an resource layer table (143), a resilience layer table (144), a measure layer table (145), a unassigned data table (146), an internet linkages table (147), a causal link table (148), an environment layer table (149), an uncertainty table (150), a resilient context space table (151), an ontology table (152), a report table (153), a reference layer table (154), a hierarchy metadata table (155), an event risk table (156), a common schema table (157), a simulations table (158), a requirement table (159), a resilient context frame table (160), a resilient context quotient table (161), a system settings table (162), a bot date table (163), a Thesaurus table (164), an id to frame table (165), a resilience model table (166), a bot assignment table (167), a scenarios table (168), a natural language table (169), a phoneme table (170), a word table (171), a phrase table (172) and a next gen sequence data table (173). The Resilient Contextbase (50) also contains a physical model library (174). The system of the present embodiment has the ability to accept and store supplemental or primary data directly from user input, a data warehouse, a virtual database, a data preparation system or other electronic files in addition to receiving data from the databases described previously. The system of the present embodiment also has the ability to complete the necessary calculations without receiving data from one or more of the specified databases.

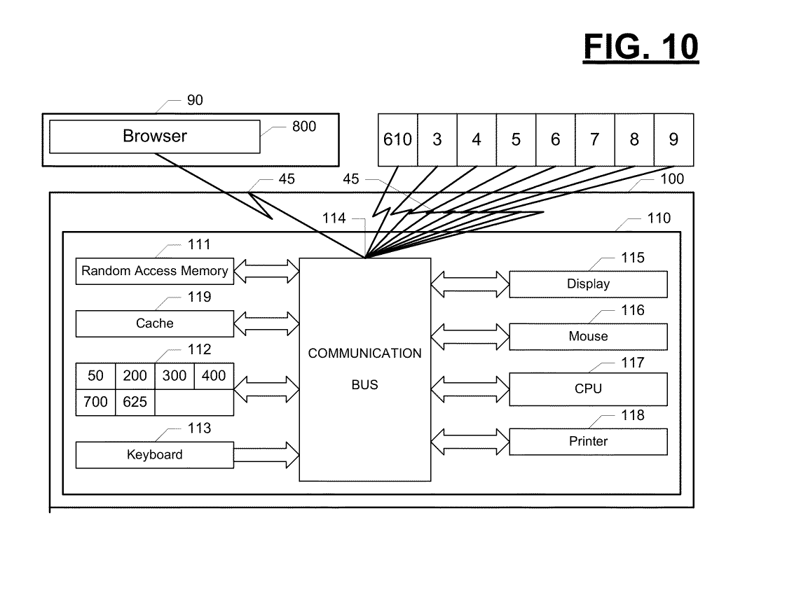

As shown in FIG. 10, one embodiment of the present embodiment is illustratively comprised of a computer (110). The computer (110) is connected via the network (45) to an internet access device (90) that contains browser software (800).

In one embodiment, the computer (110) has a read/write random access memory (111), a hard drive (112) for storage of a Resilient Contextbase (50) and the application software (200, 300, 400 and 700), a keyboard (113), a communication bus (114), a display (115), a mouse (116), a CPU (117), a printer (118) and a cache (119). As devices (3) become more capable, they may be used in place of the computer (110). Larger entities may require the use of a grid or cluster provided via a cloud based interface in place of the computer (110) to support Resilient Context Service processing requirements. In an alternate configuration, all or part of the Resilient Contextbase (50) can be maintained separately from a device (3) or computer (110) and accessed via a network (45) or grid. The computer (110) can be a personal computer running a conventional operating system, such as, e.g., Linux, Unix or Windows.

The application software (200, 300, 400 and 700) controls the performance of the central processing unit (117) as it completes the calculations used to support Resilient Context Service development. In one exemplary embodiment, the application software program (200, 300, 400 and 700) can be written in a combination of Java and C++. The application software (200, 300, 400 and 700) can use Structured Query Language (SQL) for extracting data from the databases and the World Wide Web (5, 6, 7 and 33). The user (41) can optionally interact with the user-interface portion of the application software (700) using the browser software (800) in the internet access device (90) or through a natural language interface (714) provided by the Entity Resilience System (30) to provide information to the application software (200, 300, 400 and 700).

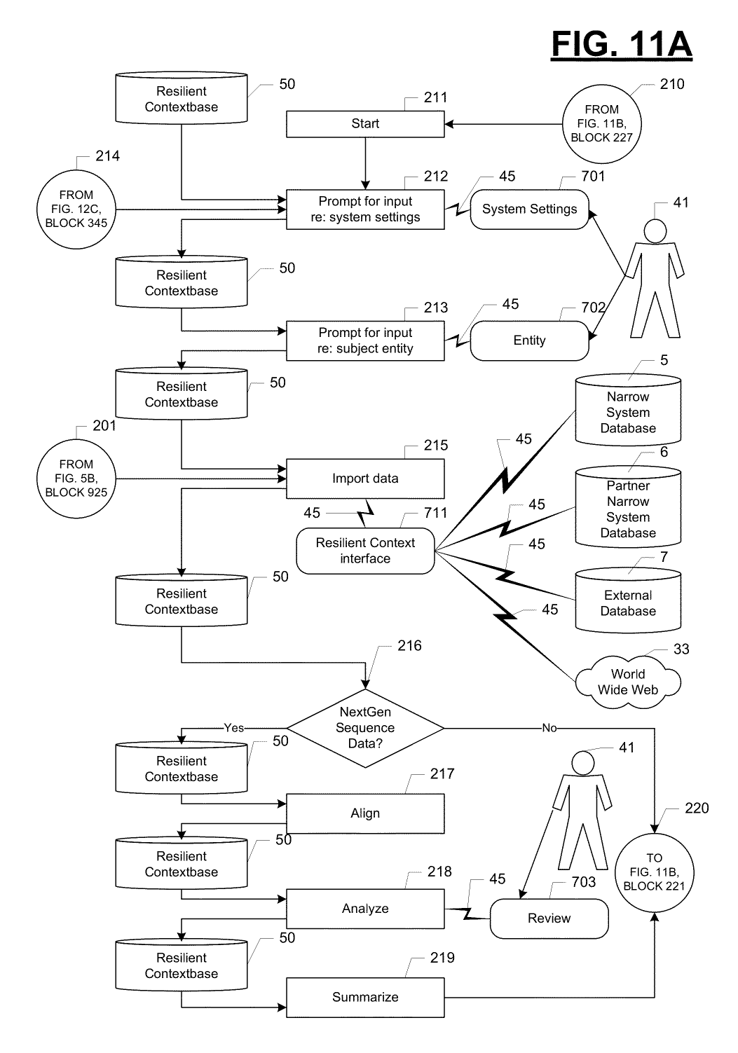

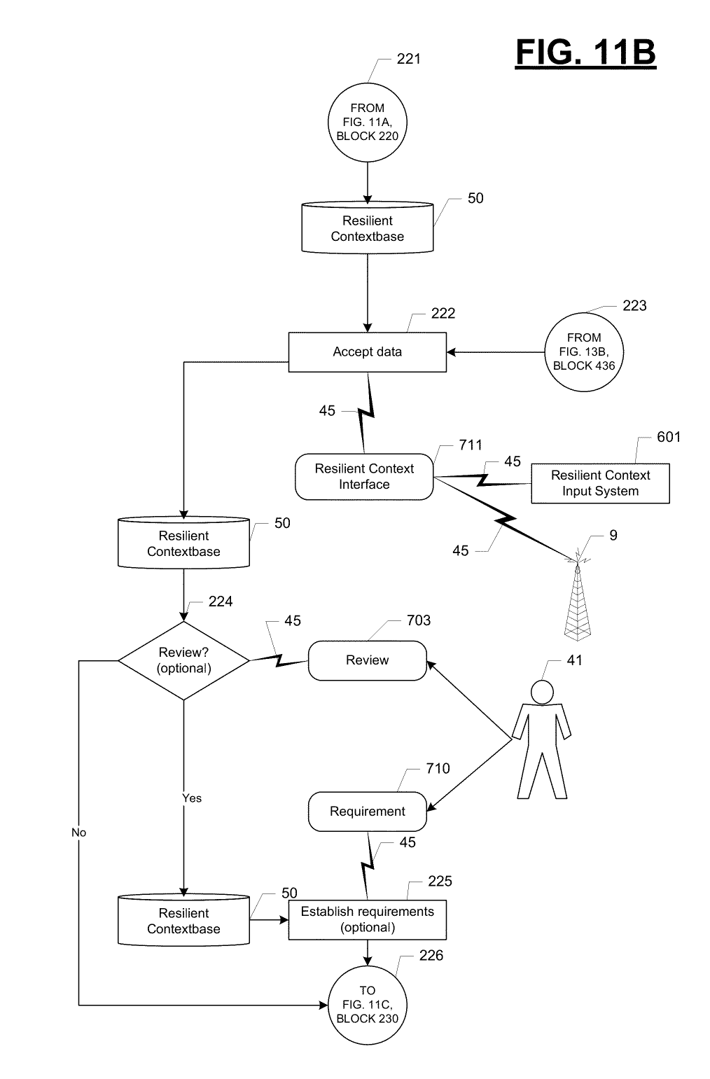

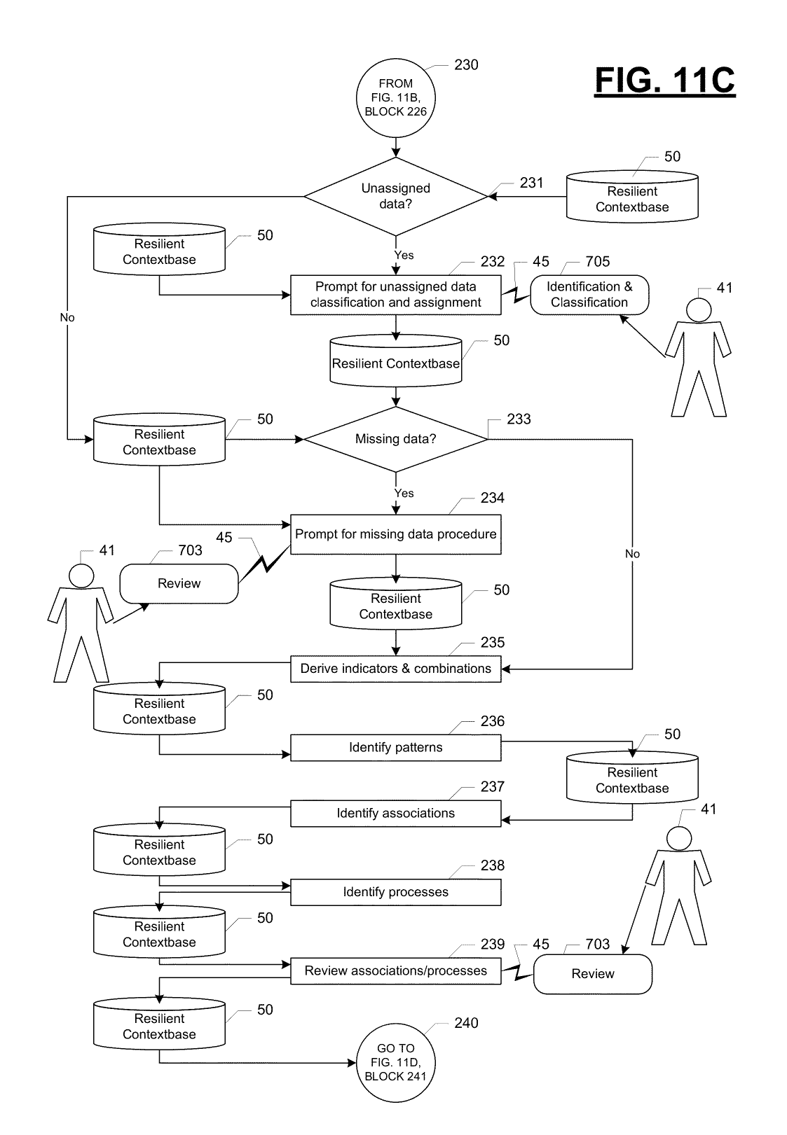

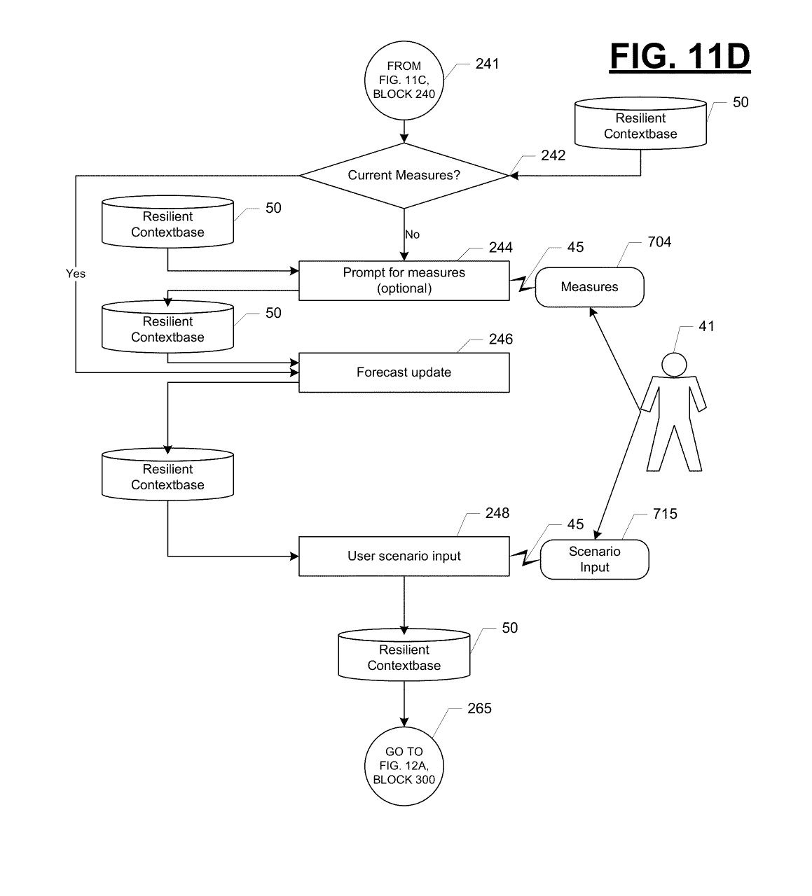

As discussed above, the Entity Resilience System (30) can complete processing in three distinct stages. As shown in FIG. 11A, FIG. 11B, FIG. 11C and FIG. 11D the first stage of processing (block 200 from FIG. 6) identifies and prepares data from narrow system databases (5); external databases (7); the world wide web (33), external services (9) and optionally, a partner narrow system database (6) for processing. This stage also identifies the entity and entity function and/or measures.

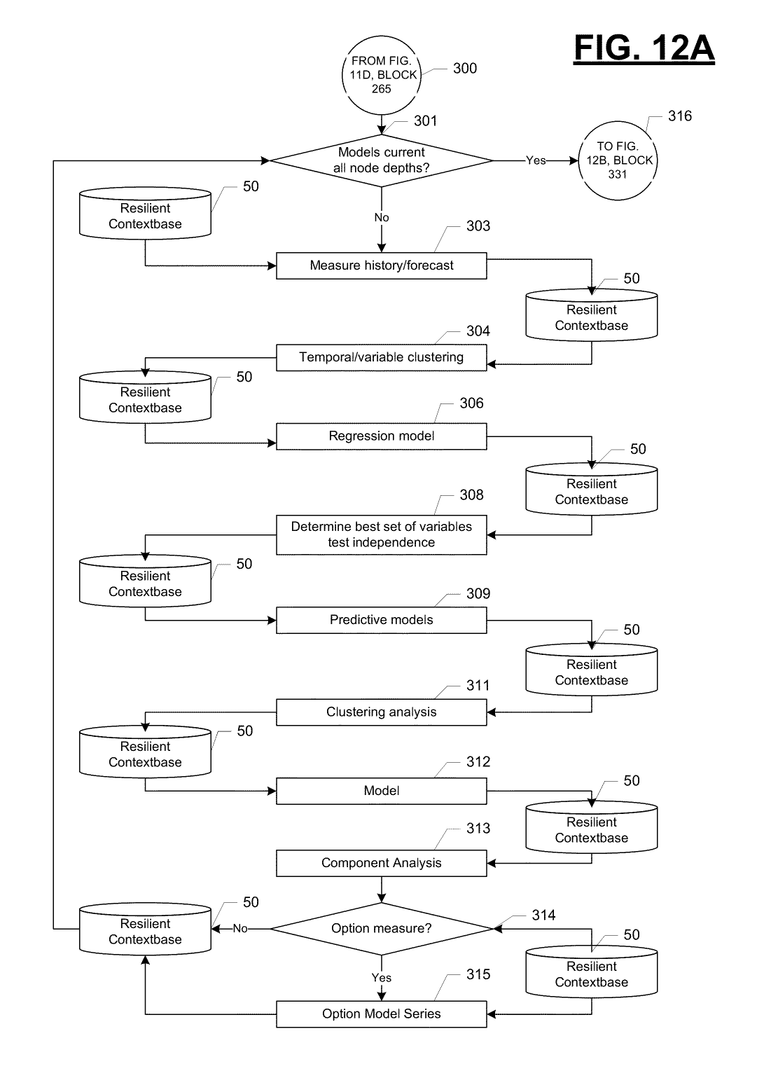

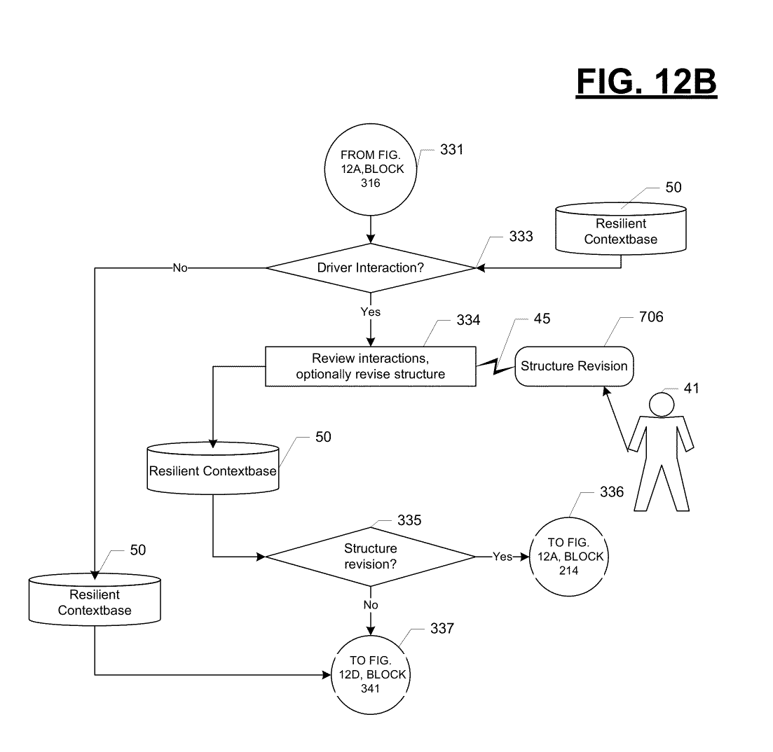

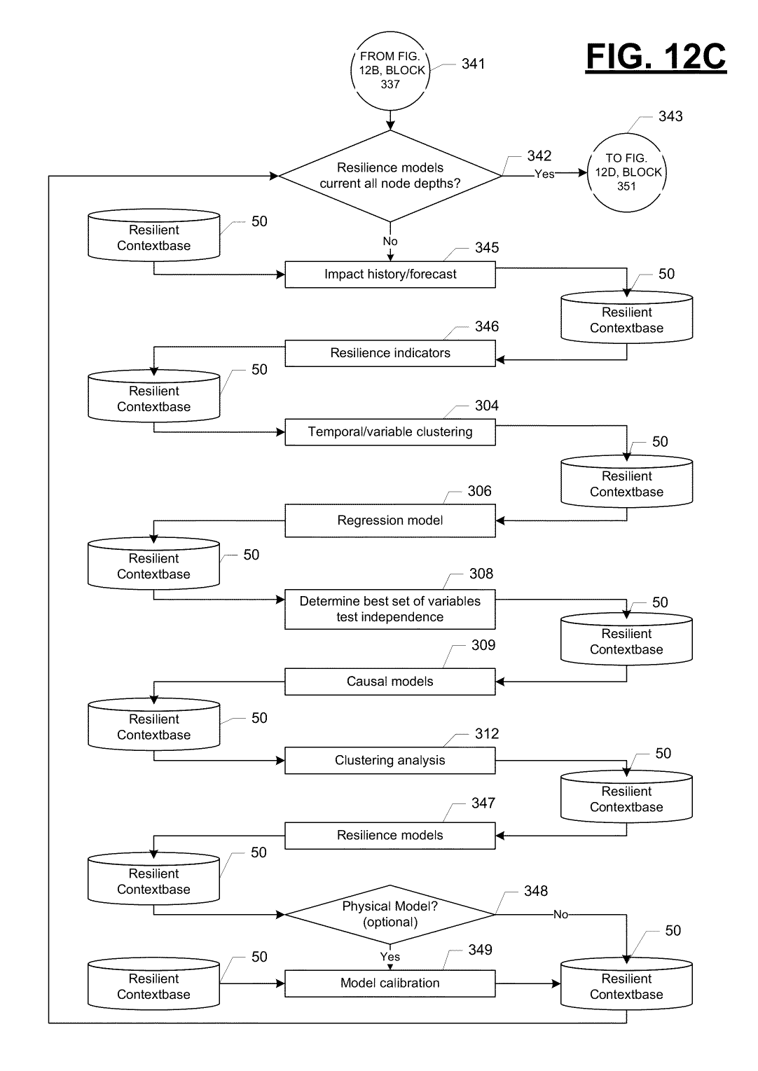

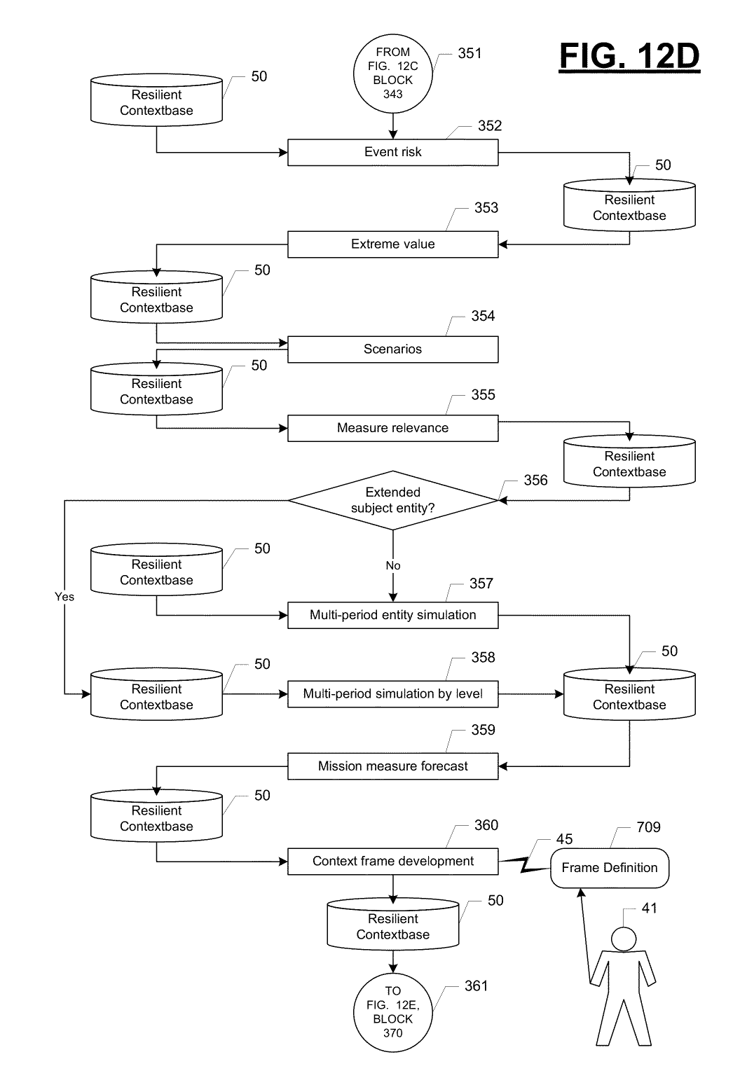

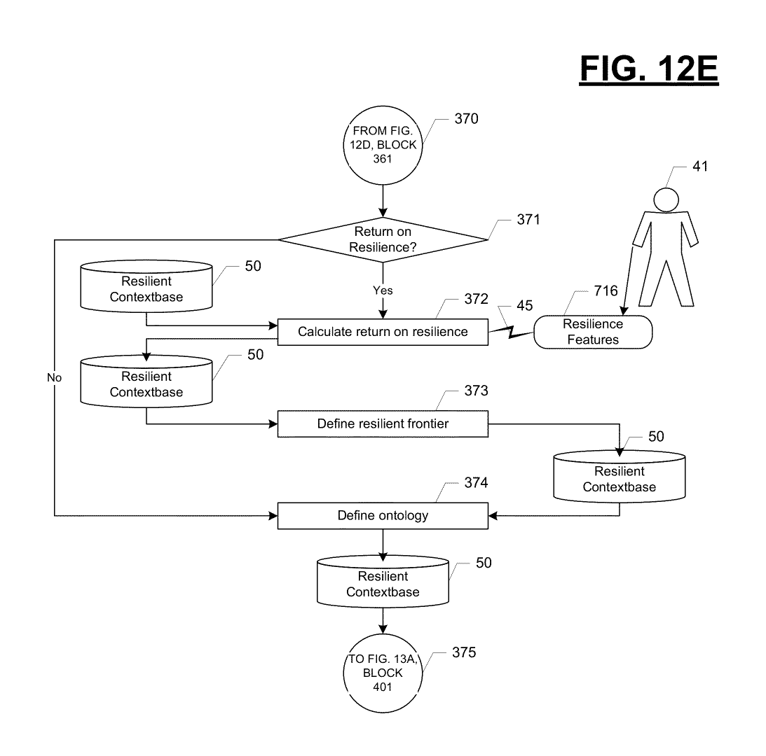

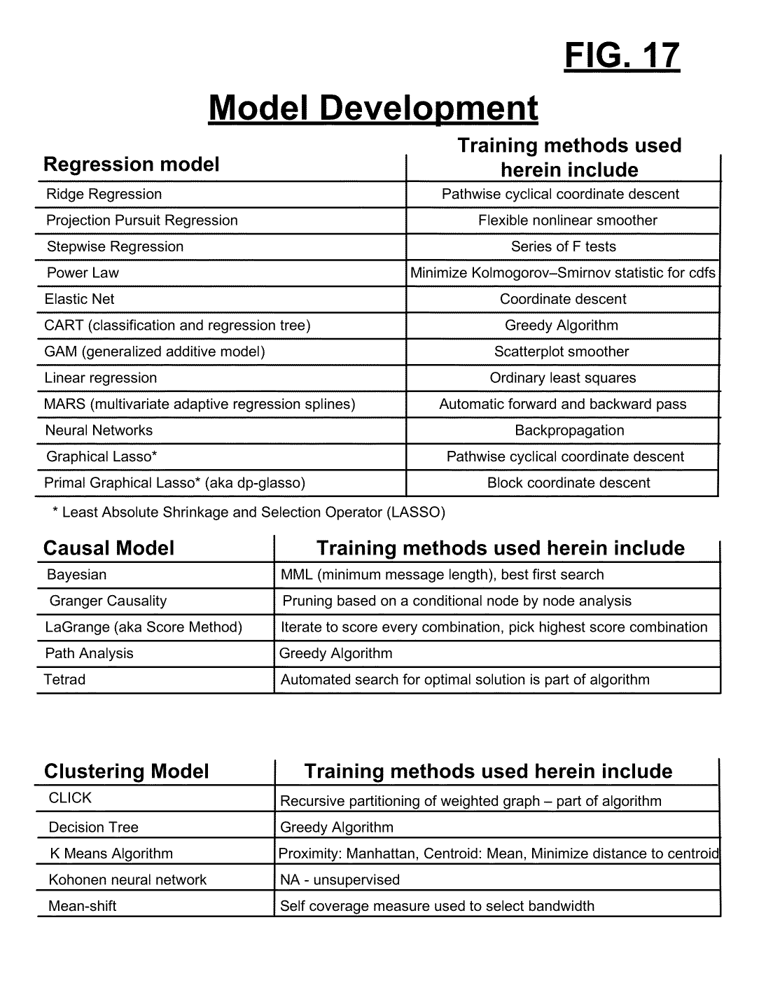

As shown in FIG. 12A, FIG. 12B, FIG. 12C, FIG. 12D and FIG. 12E, the second stage of processing (block 300 from FIG. 6) develops and then continually updates a Resilient Contextbase (50). Some of the training methods used in model development are shown in FIG. 17. In addition to using the training methods shown in FIG. 17, all predictive model development in the present embodiment involves the use of sets of training data and sets of test data. The different training data sets are created by bootstrapping which comprises re-sampling with replacement from the original training set so data records may occur more than once. The same sets of data may be used to train and then test the models developed by each type of predictive model bot.

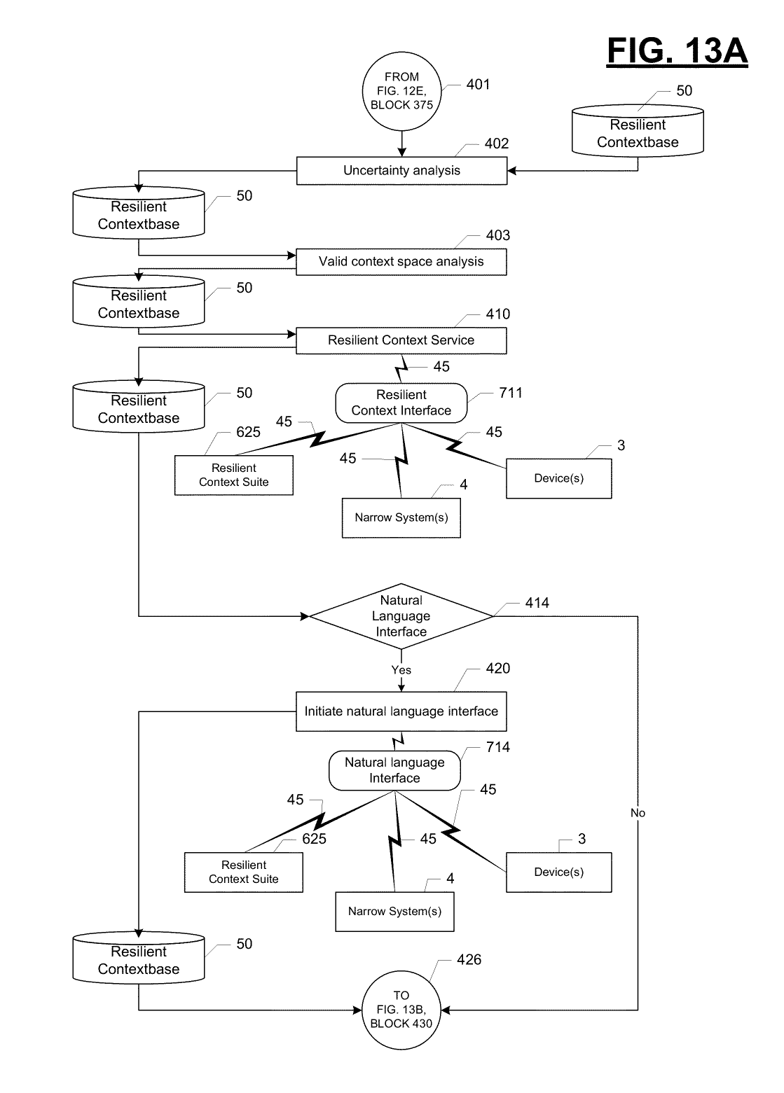

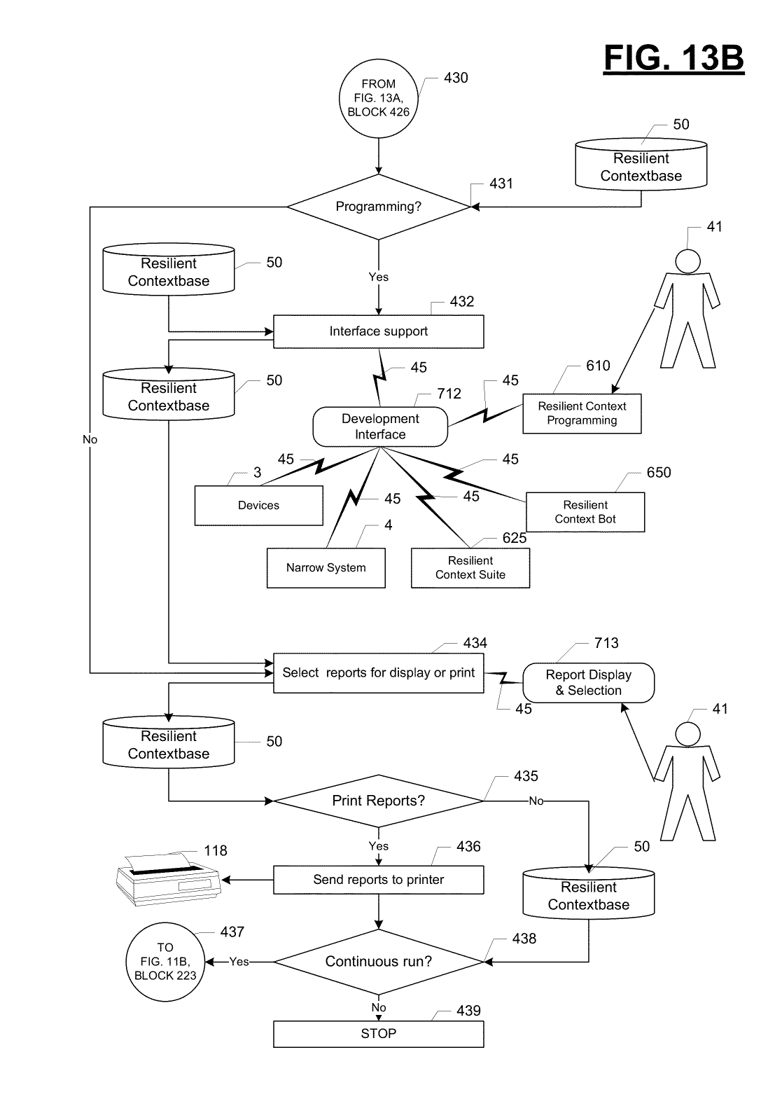

As shown in FIG. 13A and FIG. 13B, the third stage of processing (block 400 from FIG. 6) identifies the valid resilient context space before developing and distributing one or more entity contexts via an Entity Resilience System (30). The third stage of processing also prepares and prints optional reports. If the operation is continuous, then the processing steps described below are repeated continuously. As described below, one embodiment of the software is a bot or intelligent agent architecture. Those of average skill in the art will recognize that other software architectures can be used to the same effect.

### Subject Entity Definition

The flow diagrams in FIG. 11A, FIG. 11B, FIG. 11C and FIG. 11D detail the processing that is completed by the entity definition portion of the application software (200) that defines the subject entity (22), prepares data for processing and accepts user (41) input. As discussed previously, the system of the present embodiment is capable of accepting data from and transmitting data to all the narrow systems (4) listed in Tables 2, 3, 4 and 5. It can also accept data from and transmit data to the devices listed in Table 6. Operation of the Entity Resilience System (30) will be illustrated by describing the extraction and use of data from a narrow system database (5) for supply chain management and an external database (7). A brief overview of the information typically obtained from these two databases will be presented before reviewing each step of processing completed by this portion (200) of the application software.

Supply chain systems are one of the narrow systems (4) identified in Table 4. Supply chain databases are a type of narrow system database (5) that contain information that may have been in operation management system databases in the past. These systems provide enhanced visibility into the availability of resources and promote improved coordination between a subject entity (22) and its supplier entities. All supply chain systems would be expected to track all of the resources ordered by an entity after the first purchase. They typically store information similar to that shown below in Table 10.

```
TABLE 10
  1. Stock Keeping Unit (SKU)
  2. Vendor
  3. Total quantity on order
  4. Total quantity in transit
  5. Total quantity on back order
  6. Total quantity in inventory
  7. Quantity available today
  8. Quantity available next 7 days
  9. Quantity available next 30 days
10. Quantity available next 90 days
11. Quoted lead time
12. Actual average lead time
```

External databases (7) are used for obtaining information that enables the definition and evaluation of words, phrases, resilient context elements, resilient context factors and event risks. In some cases, information from these databases can be used to supplement information obtained from the other databases and the World Wide Web (5, 6 and 33). In the system of the present embodiment, the information extracted from external databases (7) includes the data listed in Table 11.

TABLE 11
1. Text information such as that found in commercial databases, such as Lexis Nexis
2. Text information from databases containing past issues of specific publications
3. Multimedia information such as video and audio clips
4. Idea market prices indicate likelihood of certain events occurring
5. Data on global event risks including information about risk probability and magnitude for weather and
geological events (e.g., Perils, EQECAT and/or ISO database, U.S. Geological Survey data re:
earthquakes)
6. Known phonemes and phrases

System processing of the information from the different data sources (3, 4, 5, 6, 7, 9 and 33) described above starts in a software block 211 that immediately advances processing to a software block 212, FIG. 11A. The software in block 212 prompts the user (41) via a system settings data window (701) to provide system setting information. The system setting information entered by the user (41) is stored in the system settings table (162) in the Resilient Contextbase (50). The specific inputs the user (41) is asked to provide at this point in processing are shown in Table 12.

```
TABLE 12
  1. Extended subject entity model? (yes or no, if yes specify node depth and cutoff criteria)
  2. Node depth for extended subject entity model
  3. Metadata standard (XML or RDF)
  4. Base currency for all pricing
  5. Source of conversion rates for currencies
  6. Continuous, if yes, calculation frequency? (by minute, hour, day, week, etc.)
  7. Standard Industrial Classification Codes (if applicable)
  8. Names of primary competitors by SIC Code (if applicable)
  9. Base account structure
10. Base units of measure
11. Base time period (default is month)
12. Base number of periods (optional, for both history and forecast data)
13. Risk free interest rate
14. Program bots or applications? (yes or no)
15. Knowledge capture and/or collaboration? (yes or no)
16. Natural language interface? (yes, no or voice activated)
17. Video data extraction? (yes or no)
18. Image data extraction? (yes or no)
19. Internet data extraction? (yes or no)
20. Reference layer? (yes or no, if yes specify coordinate system(s))
21. Text data analysis? (yes or no)
22. Geo-coded data? (if yes, then specify standard)
23. Return on Resilience Analysis? (yes or no)
24. NextGen Sequence Data? (yes or no)
25. Reference sequence(s)? (if yes, specify storage location(s))
26. Reference enterotypes? (if yes, specify storage location(s))
27. Short Oligonucleotide Analysis Package (SOAP) threshold
28. Maximum number of clusters (default is six)
29. Management report types (text, graphic or both)
30. Default missing data procedure (chose from selection-average, prior period, zero, etc.)
31. Maximum time to wait for user input
32. Maximum number of sub-elements (optional)
33. Most likely scenario: normal, extreme, user-specified or mix (default is normal)
34. System time period (days, months, years, decades, centuries, etc.)
35. Uncertainty level and source by narrow system type (optional, default is zero)
36. Weight of evidence cutoff level (by resilient context)
37. Maximum error rate for option series model (default is 10%)
38. Time frame(s) for proactive search (hours, days, weeks, etc.)
39. Node depth for scouting and/or searching for data, information and knowledge
40. Impact cutoff for scouting and/or searching for data, information and knowledge
41. How old can a model or measurement be and still be considered current?
42. Resilience measure to use (recovery time, 10% drop, 25% drop or 50% drop)
43. Use physical models to calibrate resilience models? (yes or no, default is no)
44. Use social underwriting? (yes or no)
45. Social underwriting input weighting method? (experience, risk IQ, combination or none)
46. Number of future time periods for simulations and sustainability analyses
```

The application of the remaining system settings will be further explained as part of the detailed explanation of the system operation. The software in block 212 also uses the current system date to determine the time periods (generally in months) where data will be sought to complete the calculations. The default number of time periods is 36 months of history data prior to the current system date and 24 months of forecast data after the current date. However, the user (41) also has the option of specifying the number of time periods that will be used for system calculations in the system settings table (162). After the date range for data is stored in the system settings table (162) in the Resilient Contextbase (50), processing advances to a software block 213.

The software in block 213 prompts the user (41) via an entity data window (702) to identify the subject entity (22). After the user (41) completes the specification of the subject entity, the software in block 213 selects the appropriate metadata from the hierarchy metadata table (155) and establishes the hierarchy metadata (155) and stores the ontology (152) and the common schema (157). The entity definition data are also used by the software in block 213 to establish resilient context layers. As described previously, there are generally eight types of resilient context layers for every subject entity (22). After resilient context layers are developed, the user (41) is asked to define process maps and procedures. The maps and procedures identified by the user (41) are stored in the resilience layer table (144) in the Resilient Contextbase (50). The information provided by the user (41) will be supplemented with information developed later in the first stage of processing. The Resilient Context Capture and Collaboration Service (622) can also be used here to supplement the information provided by the user (41) with information from subject matter experts (42) and/or with “social input” information. After data storage is complete, processing advances to a software block 215.

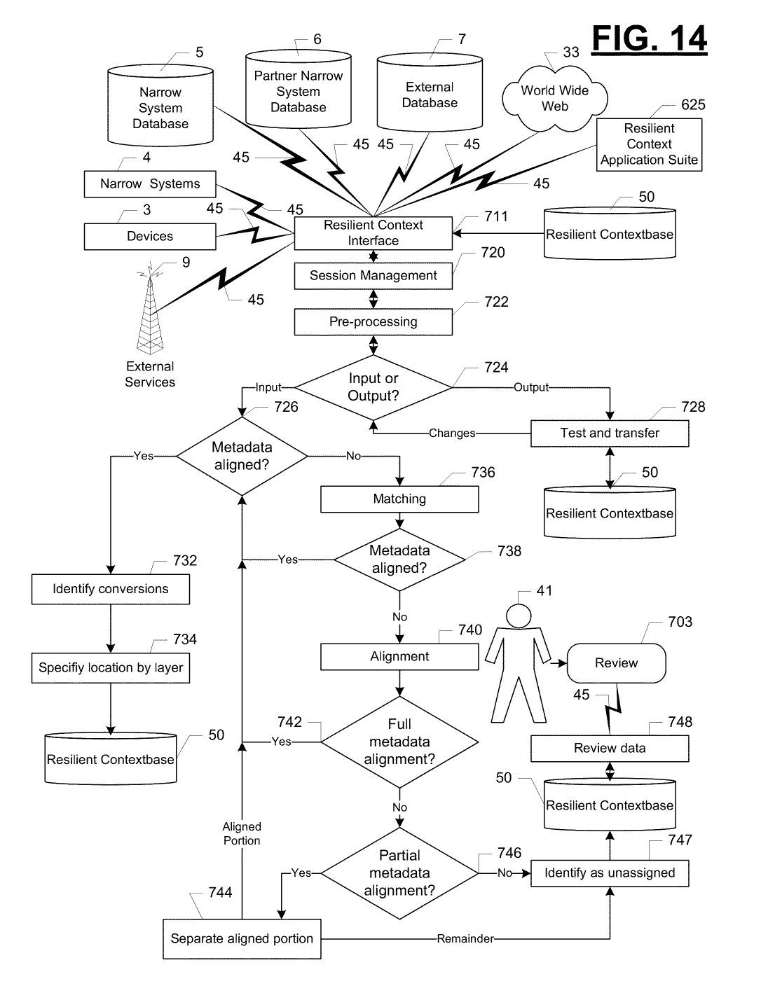

The software in block 215 uses the resilient context interface window (711) to communicate via a network (45) with the different devices (3), narrow systems (4), databases (5, 6, 7), the World Wide Web (33) and external services (9) that are data sources for the Entity Resilience System (30). As shown on FIG. 14 the resilient context interface window (711) provides access to a multiple step operation where the sequence of steps depends on the nature of the interaction and the data being provided to the Entity Resilience System (30). In one embodiment, a data input session would be managed by the a software block (720) that identifies the data source (3, 4, 5, 6, 7, 9 or 33) using standard protocols such as UDDI or XML headers, maintains security and establishes a service level agreement with the data source (3, 4, 5, 6, 7, 9 or 33). The data provided at this point could include transaction data, descriptive data, imaging data, video data, text data, sensor data, geospatial coordinate data, array data, virtual reference coordinate data and combinations thereof. The session would proceed to a pre-processing block (722) for pre-processing tasks such as discretization, transformation and/or filtering.

After completing the pre-processing in pre-processing block 722, processing would advance to a software block (724). The software in that block would determine if the data provided by the data source (3, 4, 5, 6, 7, 9 or 33) complied with the common schema or ontology using pair-wise similarity measures on several dimensions including terminology, internal structure, external structure, extensions, hierarchical classifications and semantics. If it did comply, then the data would not require alignment and the session would advance to a software block (732) where any conversions to match the base units of measure, currency or time period specified in the system settings table (162) would be identified before the session advanced to a software block (734) where the location of this data would be mapped to the appropriate resilient context layers and stored in the tables in the Resilient Contextbase (50).

As shown FIG. 14, the resilient context interface window (711) also provides access to an alternate data input processing path. This path is used if the data are not in alignment with the common schema (157) or ontology (152). In this alternate mode, the data input session would still be managed by the session management software in block (720) that identifies the data source (3, 4, 5, 6, 7, 9 or 33) maintains security and establishes a service level agreement with the data source (3, 4, 5, 6, 7, 9 or 33). The session would proceed to the pre-processing software block (722) where the data from one or more data sources (3, 4, 5, 6, 7, 9 or 33) that requires translation and optional analysis is processed before proceeding to the next step. The software in block 722 has provisions for translating, parsing and other pre-processing of audio, image, micro-array, transaction, video and unformatted text data formats to schema or ontology compliant formats (XML formats in one embodiment). Image translation involves conversion, registration, segmentation and segment identification using object boundary models. Other image analysis algorithms can be used to the same effect. Other pre-processing steps can include discretization and stochastic resonance processing.

After pre-processing is complete, the session advances to a software block 724. The software in block 724 determines whether or not the data was in alignment with the ontology (152) or the common schema (157) stored in the Resilient Contextbase (50) using pair wise comparisons as described previously. Processing then advances to the software in block 736 which uses the mappings identified by the software in block 724 together with a series of matching algorithms including key properties, similarity, global namespace, value pattern and value range algorithms to align the input data with the common schema table (157) or ontology (152).

Processing then advances to a software block 738 where the metadata associated with the data are compared with the metadata stored in the common schema table (157). If the metadata are aligned, then processing is completed using the path described previously. Alternatively, if the metadata are still not aligned, then processing advances to a software block 740 where joins, intersections and alignments between the two schemas or ontologies are completed in an automated fashion.

Processing then advances to a software block 742 where the results of these operations are compared with the common schema table (157) or ontology (152) stored in the Resilient Contextbase (50). If these operations have created alignment, then processing is completed using the path described previously. Alternatively, if the metadata are still not aligned, then processing advances to a software block 746 where the schemas and/or ontologies are checked for partial alignment. If there is partial alignment, then processing advances to a software block 744. Alternatively, if there is no alignment, then processing advances to a software block 747 where the data are tagged for manual review and stored in the unassigned data table (146). The software in block 744 cleaves the data in order to separate the portion that is in alignment from the portion that is not in alignment. The portion of the data that is not in alignment is forwarded to software block 747 where it is tagged for manual alignment and stored in the unassigned data table (146). The portion of the data that is in alignment is processed using the path described previously.

Processing advances to a software block 748 where the user (41) reviews the unassigned data table (146) using a review window (703) to see if the common schema should be modified to encompass the currently unassigned data. Changes in the common schema table (157) and/or ontology (152)—if any—are saved in the Resilient Contextbase (50). After the resilient context interface processing is completed for all available data from the devices (3), narrow systems (4), databases (5, 6 and 7), the World Wide Web (33), and external services (9), processing advances to a software block 216.

The software in block 216 checks the system settings table (162) to see if next generation sequencing data (also referred to as high throughput screening data) will be analyzed. Next generation sequencing equipment provides a platform to survey the exome, genome, microbiome, transcriptome and/or virome at a higher resolution than can be obtained using prior technologies. If, next generation sequencing data will be analyzed, then processing advances to a software block 217 If next generation sequencing data will not be analyzed, then processing advances to a software block 222. Next generation sequence data may be provided for the subject entity (22), other entities and/or for one or more resources such as air, food, water, sediment and/or soil which may contain genetic material.

The software in block 217 retrieves the reference sequence(s) from the location(s) specified in the system settings table (162) and then aligns the data stored in the nextgen sequence data table (173) with the reference sequence(s) using a bioinformatics package, such as the Short Oligonucleotide Analysis Package algorithm version 3 (Version 1 and Version 2 can also be used) after pre-processing the sequence data with the Short Read Error Reducing Aligner (SHERA) algorithm. Other algorithms such as Bowtie, Basic Local Alignment Search Tool (BLAST), Blast Like Alignment Tool (BLAT), Burrows-Wheeler Aligner (BWA), FANSe, Genomemapper, Mapping and Assembly with Quality (MAQ), MrFast, NovoAlign, Stampy, RNA Sequence Analysis Pipeline and Short Read Mapping Package (SHRiMP) can be used to the same effect. Trans-ABySS may be used for assembling and reading substrings with varying stringencies and then merging the results before analysis if there are no reference sequences. After the nextgen sequence data has been aligned to the one or more reference genomes, the aligned data are saved in the nextgen sequence data table (173) before processing advances to a software block 218.

The software in block 218 retrieves the aligned nextgen sequence data from the nextgen sequence table (173) before the Genomic Evolutionary Rate Profiling (GERP) algorithm estimates one or more constraints for each column of the alignment and identifies the constrained elements from the output for each column. A nucleosome positioning prediction engine, NuPop, then predicts nucleosome positioning using a duration hidden Markov model in which the linker DNA length is explicitly modeled. The software in the block then identifies the modules and motifs that appear to be present in the genome for each entity using the Combinatorial Algorithm for Expression and Sequence based Cluster Extraction (COALESCE) algorithm. The modules comprise elements (927) of the entity being analyzed and their identity is stored in the element layer table (141). Other algorithms such as Motif guided sparse separation algorithm or cMonkey can be used to the same effect. Separate algorithms or methods for identifying modules and for identifying motifs may also be used in place of the integrated analysis of modules and motifs. After the modules and motifs are identified, they are compared to any reference modules and motifs that may have been provided and the variance will be noted. The software in block 218 also allows the user (41) to identify variants in the aligned genome with the genome analysis tool kit (GATK) that incorporates the Dindel algorithm. Other tools for identifying variants such as ANNOVAR and BEDTools can also be used to the same effect. If a Bina Box has been used as a data source, then the variance analysis from that system can also be used as an input. If data from more than one generation is available, then the “identify by descent” (IBD) or fast identity by descent (fastIDB) algorithms can also be used to complete analyses. If the nextgen sequence data comprises bacteria data from the subject entity (22) microbiome, then the software in this block will also compare the data to the reference enterotypes in order to identify the enterotype of each microbiome population. Variation from the mix of bacteria found in the identified reference enterotype is also be calculated and saved. For example, if the reference enterotype contained 33.33% Bacteria A, 33.33% Bacteria B and 33.34% Bacteria C and the subjects microbiome contained 50% Bacteria A, 25% Bacteria B and 25% Bacteria C, then the variance of +16.67% for Bacteria A, −8.33% for Bacteria B and −8.34% for Bacteria C would be calculated and stored. The identified sequence variants, enterotype, variations in enterotype mix and observed virome mix are then stored in the nextgen sequence data table (173) before processing advances to software block 219.

The software in block 219 retrieves the information from the nextgen sequence data table (173) and creates a summary identifying the subject entity (22) genome by module, the subject entity (22) genomic variants by module and gene, the enterotype classification of the subject's microbiome, the subject's microbiome mix of bacteria, the variation in the subject's microbiome mix from the enterotype mix (see preceding paragraph for example calculation) and the subject's virome mix (if any). A similar summary can also be created for other entities. These genomic summaries comprise additional information regarding the subject entity (22) while the microbiome and virome related summaries comprise factors in a definition of the entity in the expanded subject entity (22) system being modeled and analyzed by the Entity Resilience System (30). These summaries are saved in the system settings table (162) and in the Resilient Contextbase. If nextgen sequence data have been provided for resources, then the software in block 219 retrieves the information from the nextgen sequence data table for the resources (173) and creates a summary identifying the mix of life forms present in each resource, the variation in the mix from the reference mix (if available) as well as any variations in the genetic material in said life forms from the reference genome at the gene and module level. The summaries associated with the resources are saved in the resource layer table (143) in the Resilient Contextbase. After the summaries are saved, processing advances to a software block 222.

The software in block 222 optionally prompts the resilient context interface window (711) to communicate via a network (45) with the Resilient Context Input System (601). The resilient context interface window (711) uses the path described previously for data input to map any data input to the appropriate resilient context layers and store the data in the Resilient Contextbase (50) as described previously. After storage of the Resilient Context Input System (601) data are complete, processing advances to a software block 224.

The software in block 224 prompts the user (41) via the review window (703) to optionally review the resilient context layer data that has been stored in the first few steps of processing. The user (41) has the option of changing the data on for a single use or permanently. Any changes the user (41) makes are stored in the table for the corresponding resilient context layer (e.g., transaction layer changes are saved in the transaction layer table (142), etc.). As part of the processing in this block, an interactive GEL algorithm prompts the user (41) via the review data window (703) to check the hierarchy or group assignment of any new elements, factors and resources that have been identified. Any newly defined categories are stored in the resilience layer table (144) and the common schema table (157) in the Resilient Contextbase (50) before processing advances to a software block 225.

The software in block 225 prompts the user (41) via a requirement data window (710) to optionally identify requirements for the subject. Requirements can take a variety of forms but the two most common types of requirements are absolute and relative. For example, a requirement that the level of cash should never drop below $50,000 is an absolute requirement while a requirement that there should never be less than two months of cash on hand is a relative requirement. The requirement data window ((710) also allows the user (41) to establish categories for the different requirements. These categories can be used in the Resilient Context Compliance Service (626) to report on different categories of requirements with different frequencies. Examples of different requirements are shown in Table 13.

TABLE 13

| Entity | Requirement (reason) |
|---|---|
| Individual | Stop working at 67 (retirement) |
| (1301) | Keep blood pressure below 155/95 (health) |
| | Available funds >$X by Jan. 1, 2014 (college for daughter) |
| Circulatory | Cholesterol level between 120 and 180 |
| System | Blood pressure between 110/75 and 150/100 |
| (2303) | |

The software in this block provides the ability to specify absolute requirements, relative requirements and standard “requirements” for any reporting format that is defined for use by the Resilient Context Review Service (607). After requirements are specified, they are stored in the requirement table (159) in the Resilient Contextbase (50) by entity before processing advances to a software block 231.

The software in block 231 checks the unassigned data table (146) in the Resilient Contextbase (50) to see if there are any data that have not been assigned to an entity and/or resilient context layer. If there are no data without a complete assignment (an entity or resilient context layer assignment constitutes a complete assignment), then processing advances to a software block 233. Alternatively, if there are data without an assignment, then processing advances to a software block 232. The software in block 232 prompts the user (41) via an identification and classification data window (705) to identify the resilient context layer and entity assignment for the data in the unassigned data table (146). After assignments have been specified for every data element, the resulting assignments are stored in the appropriate resilient context layer tables in the Resilient Contextbase (50) by entity before processing advances to a software block 233.

The software in block 233 checks the element layer table (141), the transaction layer table (142) and the resource layer table (143) and the environment layer table (149) in the Resilient Contextbase (50) to see if data are missing for any specified time period. If data are not missing for any time period, then processing advances to a software block 235. Alternatively, if data for one or more of the specified time periods identified in the system settings table (162) for one or more items is missing from one or more resilient context layers, then processing advances to a software block 234. The software in block 234 prompts the user (41) via the review data window (703) to specify the procedure that will be used for generating values for the items that are missing data by time period. Options the user (41) can choose at this point include: the average value for the item over the entire time period, the average value for the item over a specified time period, zero or the average of the preceding item and the following item values and direct user input for each missing value. If the user (41) does not provide input within a specified interval, then the default missing data procedure specified in the system settings table (162) is used. When the missing time periods have been filed and stored for all the database fields that were missing data, then system processing advances to a software block 235.

The software in block 235 retrieves data that was not obtained from one or more nextgen sequencing systems from the element layer table (141), the transaction layer table (142), the resource layer table (143) and the environment layer table (149). It uses this data to calculate indicators for the data associated with each element, resource and environmental factor. The calculation of indicators from the next gen sequencing data was previously described with respect to software blocks 216 through 219. The indicators calculated in this step are comprised of comparisons, regulatory measures and statistics. Comparisons and statistics are derived for: appearance, description, numeric, shape, shape/time and time characteristics. These comparisons and statistics are developed for different types of data as shown below in Table 14.

TABLE 14

| Charac- | | | | | | |
|---|---|---|---|---|---|---|
| teristic/ | | | | | Shape- | |
| Data type | Appearance | Description | Numeric | Shape | Time | Time |
| audio | | X | X | | | X |
| coordinate | | X | X | X | X | X |
| image | X | | X | X | X | X |
| text | | X | X | | | X |
| transaction | | | X | | | X |
| video | X | | X | X | X | X |

X = comparisons and statistics are developed for these characteristic/data type combinations

Numeric characteristics are pre-assigned to different domains. Numeric characteristics include amperage, area, concentration, density, depth, distance, growth rate, hardness, height, hops, impedance, level, mass to charge ratio, nodes, quantity, rate, resistance, similarity, speed, tensile strength, voltage, volume, weight and combinations thereof. Time characteristics include frequency measures, gap measures (e.g., time since last occurrence, average time between occurrences, etc.) and combinations thereof. The numeric and time characteristics can also be combined to calculate additional indicators using the LINUS algorithm. Comparisons include: comparisons to baseline (can be binary, 1 if above, 0 if below), comparisons to external expectations, comparisons to forecasts, comparisons to goals, comparisons to historical trends, comparisons to known bad, comparisons to known good, life cycle comparisons, comparisons to normal, comparisons to peers, comparisons to regulations, comparison to requirements, comparisons to a standard, sequence comparisons, comparisons to a threshold (can be binary, 1 if above, 0 if below) and combinations thereof. Statistics include: averages (mean, median and mode), convexity, copulas, correlation, covariance, derivatives, Pearson correlation coefficients, slopes, trends and variability. Time lagged versions of each piece of data, statistic and comparison are also developed. The numbers derived from these calculations are collectively referred to as “indicators” (also referred to as item performance indicators and factor performance indicators). The indicators are stored in the appropriate resilient context layer table—the element layer table (141), the resource layer table (143) or the environment layer table (149)—before processing advances to a software block 236.

The software in block 236 checks the bot date table (163) and deactivates pattern bots with creation dates before the current system date and retrieves information from the system settings table (162), the element layer table (141), the transaction layer table (142), the resource layer table (143) and the environment layer table (149). The software in block 236 then initializes pattern bots for each layer to identify patterns in that data stored in each layer. Bots are independent components of the application software of the present embodiment that complete specific tasks. In the case of pattern bots, their tasks are to identify patterns in the data associated with each resilient context layer. In one embodiment, pattern bots use Apriori algorithms to identify patterns including frequent patterns, sequential patterns and multi-dimensional patterns. However, a number of other pattern identification algorithms including the sliding window algorithm; differential association rule, beam-search, frequent pattern growth, decision trees and the PASCAL algorithm can be used alone or in combination to the same effect. Every pattern bot contains the information shown in Table 15.

TABLE 15
1. Unique ID number (based on date, hour, minute, second of creation)
2. Creation date (date, hour, minute, second)
3. Storage location
4. Entity type(s)
5. Subject entity
6. Resilient context layer
7. Algorithm

After being initialized, the bots identify patterns for the data associated with elements, resources, factors and combinations thereof. Each pattern is given a unique identifier and the frequency and type of each pattern is determined. The numeric values associated with the patterns are indicators. The values are stored in the appropriate resilient context layer table before processing advances to a software block 237.

The software in block 237 uses causal association algorithms such as local causal discovery (LCD) to identify causal associations between indicators, composite variables, element data, factor data, resource data and events, actions, processes and measures. The LCD algorithm determines if CCC and/or CCU causality associations are present in the data. The CCC causality rule is as follows: If A, B and C are three variables that are pair wise correlated (CCC—all three pairs (A, B), (B, C) and (A, C) are correlated) and A and C become independent when conditioned on B. The CCU causality rule is as follows: If A, B and C are three variables such that (A, B) and (A, C) are correlated and (B, C) are uncorrelated (CCU—two pairs are correlated and one pair is uncorrelated) and B and C become dependent when conditioned on A. The software in this block also uses semantic association algorithms including path length, subsumption, source uncertainty and resilient context weight algorithms to identify semantic associations. The identified associations are stored in the causal link table (148) before processing advances to a software block 238.

The software in block 238 uses a tournament of petri nets, time warping algorithms and stochism algorithms to identify probable subject entity (22) processes in an automated fashion. Other pathway identification algorithms can be used to the same effect. The identified processes are stored in the element layer table (141) before processing advances to a software block 239. The software in block 239 prompts the user (41) via the review data window (703) to optionally review the new associations stored in the causal link table (148) and the newly identified processes stored in the element layer table (141). Associations and/or processes that have already been specified or approved by the user (41) will not be displayed automatically. The user (41) has the option of accepting or rejecting each identified association or process. Any associations or processes the user (41) accepts are stored in the element layer table (141) before processing advances a software block 242.

The software in block 242 checks the measure layer table (145) in the Resilient Contextbase (50) to determine if there are current models for all measures for every entity. If all measure models are current for every entity, then processing advances to a software block 246. Alternatively, if all measure models are not current, then processing advances to a software block 244.

The software in block 244 prompts the user (41) via a measures data window (704) to optionally specify a new mission measure for the subject entity (22), optionally specify new function measures for the subject entity, optionally specify new function measures for subject entity systems, optionally specify new function measures for subject entity organs by system and to optionally specify new function measures for subject entity cells by organ and system. Because maintaining subject entity health is the default mission, the default measure is the Quality of Well Being (QWB) health measure. The Quality of Well-Being (QWB) Scale measures quality of life by determining the objective levels of an individual's functioning in three domains: mobility, physical activity, and social activity. In addition to these three domains, the QWB Scale also assesses a wide variety of symptoms. The QWB Scale measures functional performance rather than functional ability: the subject is asked to report activity that has actually been performed, as opposed to activity that the subject thinks that they could hypothetically perform. The QWB Scale is a good measure of outcomes of serious illness over time. Scoring/Interpretation: Each of the three domain scales is weighted. Overall scores range from 0 to 1.0 with a higher score representing a better state of health. A score of zero indicates death while a score of 1.0 indicates asymptomatic optimum functioning. Other health measures such as the Health Utilities Index (HUI) and the EuroQol Instrument (EQ-5D) index could be used to the same effect. The default function measures for the subject entity systems, organs and cells are shown in FIG. 20.

As detailed below, the history of the underlying source(s) of uncertainty for any option measures are analyzed using the same procedure used for analyzing the other measures. As discussed previously, the user (41) is given the option of using pre-defined measures or creating new measures using terms defined in the common schema table (157). The measures can combine performance and risk measures or the performance and risk measures can be kept separate. If more than one measure is defined for the subject entity (22), then the user (41) is prompted to assign a weighting or relative priority to the different measures that have been defined. As system processing advances, the assigned priorities can be compared to the priorities that entity actions indicate are most important. The priorities used to guide analysis can be the stated priorities, the priorities inferred from the analysis of subject entity actions or some combination thereof. The gap between stated priorities and actual priorities is a congruence measure that can be used in analyzing aspects of performance.

After the optional specification of measures and priorities has been completed, the values of each of the newly defined measures are calculated using historical data and forecast data. If forecast data are not available, then the Resilient Context Forecast Service (603) is used to supply the missing values. These values are then stored in the measure layer table (145) along with the measure definitions and priorities. When data storage is complete, processing advances to a software block 246.

The software in block 246 checks the bot date table (163) and deactivates forecast update bots with creation dates before the current system date. The software in block 256 then retrieves the information from the system settings table (162) and environment layer table (149) in order to initialize forecast bots in accordance with the frequency specified by the user (41) in the system settings table (162). Bots are independent components of the application software that complete specific tasks. In the case of forecast update bots, their task is to compare the forecasts values for data stored in the Resilient Contextbase (50) with the information available from public futures exchanges. This function is generally only used when the system is not run continuously. Every forecast update bot activated in this block contains the information shown in Table 16.

TABLE 16
1. Unique ID number (based on date, hour, minute, second of creation)
2. Creation date (date, hour, minute, second)
3. Mapping information
4. Storage location
5. Subject entity
6. Resilient Context factor
7. Measure
8. Forecast time period

After the forecast update bots are initialized, they activate in accordance with the frequency specified by the user (41) in the system settings table (162). Once activated, they retrieve the specified information and determine if any forecasts need to be updated to bring them in line with the most current data. The bots save the updated forecasts in the appropriate table in the Resilient Contextbase (50) by entity and processing advances to a software block 248.

The software in block 248 prompts the user (41) via a scenario input window (715) to specify one or more scenarios for the subject entity. The user (41) may also specify one or more scenarios for related entities. The scenarios comprise forecasts of element, factor or resource levels and/or outputs for a number of time periods in the future. The scenarios may also include forecast of the underlying source(s) of uncertainty for an option measure. After the user completes the specification of one or more scenarios, the scenarios are saved in the scenarios table (168) by entity in the Resilient Contextbase (50) and processing advances to a software block 301.

### Resilient Contextbase Development

The flow diagrams in FIG. 12A, FIG. 12B, FIG. 12C, FIG. 12D and FIG. 12E detail the processing that is completed by the portion of the application software (300) that continually develops a mission oriented Resilient Contextbase (50) by creating and activating analysis bots that:
1. Identify the impact of the elements, factors, resources, events, actions on subject entity function measures, on subject entity resilience and on the subject entity mission (maintaining health is the default mission) by learning from the data;
2. Develop the measure layer (145) by transforming data into robust models of the elements, factors, resources, events, actions, one or more function measures and a health measure by learning from the data;
3. Develop the resilience layer (144) by transforming data into robust models of subject entity resilience by learning from the data, and
4. Determine the relationship between function measure performance, resilience and subject entity mission (maintaining health is the default mission) by learning from the data.

Each analysis bot normalizes the data being analyzed before processing begins. The system of the present embodiment can combine any number of measures in order to evaluate the performance of any entity in the hierarchies/groups described previously. As discussed previously, the default measure is the QWB and the default functions measures are measures of mobility, physical activity and social activity.

Before discussing this stage of processing in more detail, it will be helpful to review the processing already completed. As discussed previously, the Resilient Context is being developed for the subject entity (22) by developing a detailed understanding of the impact of elements, factors, resources, events, actions and other entities on one or more subject entity function measures and subject entity resilience. Some of the elements and resources may have been grouped together to complete processes (a special class of element). The first stage of processing reviewed the data from some or all of the narrow systems (4) listed in Tables 2, 3, 4 and 5 and the devices (3) listed in Table 6 and then established a Resilient Contextbase (50) that formalized the definition of the identity and description of the elements, factors, resources, events and transactions that impact subject entity (22) function measure performance and resilience. The Resilient Contextbase (50) also ensures ready access to the data used for the second and third stages of computation in the Entity Resilience System (30). In the second stage of processing, the Resilient Contextbase (50) is used to develop an understanding of the relative impact of the different elements, factors, resources, events and transactions on subject entity function measures, resilience and mission.

Processing in this portion of the application begins in software block 301. The software in block 301 checks the measure layer table (145) in the Resilient Contextbase (50) to determine if there are current models for all function measures and for all underlying source(s) of uncertainty for any option measures for all node depths identified in the system settings table (162). Measures that combine a performance measure and a risk measure into a single measure are considered two measures for purposes of this evaluation. If all models are current for all the node depth levels identified in the system settings table, then processing advances to a software block 333. Alternatively, if all measure models are not current for all node depth levels, then processing advances to a software block 303. As discussed previously, the default function measures are measures of mobility, physical activity, and social activity and the node depth level defines the number of type of analyses that should be completed. The number and type of models developed by this portion of the application software is a function of the node depth that has been specified in the system settings table as shown in Table 17.

TABLE 17

| Node | Number of models | | Output variables |
|---|---|---|---|
| depth | developed | Inputs | (default) |
| 1 | Three (one for | Characteristic and function measure data and | 1.mobility measure, |
| | each function | indicators at system, organ, cell and genetic | 2. physical activity |
| | measure) | material level by system; characteristic and | measure and 3. social |
| | | function measure data and indicators by | activity measure |
| | | resource entity; characteristic and function | |
| | | measure data and indicators by non-biological | |
| | | element and environmental entity | |
| 2 | All models from | Characteristic and function measure data and | Contribution of each |
| | node depth 1 plus | indicators at organ, cell and genetic material | organ to each system |
| | a model for each | levels by organ; characteristic and function | model |
| | specified organ to | measure data and indicators by resource entity; | |
| | each of 14 systems | characteristic and function measure data and | |
| | | indicators by non-biological element and | |
| | | environmental entity | |
| 3 | All models from | Characteristic and function measure data and | Contribution of each |
| | node depth 2 plus | indicators at cell and genetic material levels by | cell to each organ |
| | a model for each | cell type; characteristic and function measure | model |
| | type of cell to each | data and indicators by resource entity; | |
| | specified organ | characteristic and function measure data and | |
| | | indicators by non-biological element and | |
| | | environmental entity | |
| 4 | All models from | Characteristic and function measure data and | Contribtion of each |
| | node depth 3 (see | indicators at genetic material level by genetic | piece of genetic |
| | FIG. 20) | material type; characteristic and function | material to each cell |
| | | measure data and indicators by resource entity; | model |
| | | characteristic and function measure data and | |
| | | indicators by non-biological element and | |
| | | environmental entity | |

The software in block 303 retrieves the values for the next measure (or underlying source of uncertainty for an option measure) for prior periods and future periods from the measure layer table (145) before processing advances to a software block 304. The software in block 304 checks the bot date table (163) and deactivates temporal and variable clustering bots with creation dates before the current system date. The software in block 304 then initializes temporal clustering bots in accordance with the frequency specified by the user (41) in the system settings table (162). The bots retrieve information from the measure layer table (145) for the entity being analyzed and defines regimes for the measure being analyzed before saving the resulting cluster information in the measure layer table (145) in the Resilient Contextbase (50). Bots are independent components of the application software of the present embodiment that complete specific tasks. In the case of temporal clustering bots, their primary task is to segment measure levels into distinct time regimes that share similar characteristics. The temporal clustering bots also identify distinct time regimes for the underlying source(s) of uncertainty for the option measures. The temporal clustering bot assigns a unique identification (id) number to each “regime” it identifies before tagging and storing the unique id numbers in the measure layer table (145). Every time period with data is assigned to one of the regimes. The cluster id for each regime is associated with the measure and entity being analyzed. The time regimes are developed using a competitive regression algorithm that identifies an overall, global model before splitting the data and creating new models for the data in each partition. If the average relative root mean squared error from the two models is greater than the average relative root mean squared error from the global model, then there is only one regime in the data. Alternatively, if the two models produce lower average relative root mean squared error than the global model, then a third model is created. If the error from three models is lower than from two models then a fourth model is added. The processing pattern described in the preceding sentences continues until adding a new model does not improve accuracy. Every temporal clustering bot contains the information shown in Table 18.

TABLE 18
1. Unique ID number (based on date, hour, minute, second of creation)
2. Creation date (date, hour, minute, second)
3. Mapping information
4. Storage location
5. Maximum number of clusters
6. Subject entity
7. Node depth being modeled (1, 2, 3 or 4)
8. Function measure or underlying source of uncertainty for an option measure

The temporal clustering bots identify and store regime assignments for all historical and forecast time periods in the measure layer table (145). The software in block 304 also initializes variable clustering bots for data associated with each element, resource and factor. The variable clustering bots activate in accordance with the frequency specified by the user (41) in the system settings table (162), retrieve the information from the element layer table (141), the transaction layer table (142), the resource layer table (143), the environment layer table (149) and the common schema table (157) before identifying segments or clusters for element, resource and factor data and then tagging and saving the resulting cluster information in the appropriate table. Bots are independent components of the application software of the present embodiment that complete specific tasks. In the case of variable clustering bots, their primary task is to segment the element, resource and factor data—including performance indicators—into distinct clusters that share similar characteristics. The variable clustering bots assign a unique id number to each “cluster” they identify. The unique id numbers for the element clusters are stored at the item variable level in the element layer table (141). The unique id numbers for the resource clusters are stored at the item variable level in the resource layer table (143). The unique id numbers for the factor clusters are stored at the item variable level in the environment layer table (149). Every item variable for each element, resource and factor is assigned to one of the unique clusters. The element data, resource data and factor data are segmented into a number of clusters less than or equal to the maximum specified by the user (41) in the system settings table (162). The data are segmented using mean shift clustering. Several other clustering algorithms including: an unsupervised “Kohonen” neural network, decision tree, CLICK-Cluster Identification via Connectivity Kernels and the K-means algorithm can be used to the same effect. For algorithms that normally use the specified number of clusters as part of processing, the variable clustering bots uses the maximum number of clusters specified by the user (41) in the system settings table (162). Every variable clustering bot contains the information shown in Table 19.

TABLE 19
1. Unique ID number (based on date, hour, minute, second of creation)
2. Creation date (date, hour, minute, second)
3. Mapping information
4. Storage location
5. Resilient context component (element, factor or resource)
6. Clustering algorithm type
7. Subject entity
8. Node depth being modeled (1, 2, 3 or 4)
9. Maximum number of clusters

When the variable clustering bots have identified, tagged and stored cluster assignments for the data associated with every element, resource and factor in the appropriate table, processing advances to a software block 306.

The software in block 306 checks the bot date table (163) and deactivates all regression model bots with creation dates before the current system date. The software in block 306 then retrieves the information from the measure layer table (145), the common schema table (157), the element layer table (141), the transaction layer table (142), the resource layer table (143) and the environment layer table (149) in order to initialize regression model bots for the current measure (or underlying source of uncertainty for an option measure). Bots are independent components of the application software that complete specific tasks. In the case of regression model bots, their primary task is to develop a regression model for the measure being evaluated that uses the indicators and the item variables from the elements, resources and factors as inputs. A primal graphical LASSO (dp-glasso) algorithm is used to identify the relevant input variables and develop a regression model for the measure. Every regression model bot contains the information shown in Table 20.

TABLE 20
1. Unique ID number (based on date, hour, minute, second of creation)
2. Creation date (date, hour, minute, second)
3. Mapping information
4. Storage location
5. Subject entity
6. Node depth being modeled (1, 2, 3 or 4)
7. Function measure, underlying source of uncertainty for an option measure or component of context

After regression model bot is initialized, the bot activates in accordance with the frequency specified by the user (41) in the system settings table (162). Once activated, the bot retrieves the specified data from the appropriate table in the Resilient Contextbase (50) and randomly partition the element, resource or factor data into a training set and a test set. A software block 308 then uses “bootstrapping” where different training data sets are created by re-sampling with replacement from the original training set so data records may occur more than once. After the regression model bots complete their training and testing using the bootstrapped data sets and the training method identified in FIG. 17, the data used as inputs to the best fit regression model for the measure (or underlying source of uncertainty for an option measure) are identified as performance drivers for that measure or underlying source of uncertainty for an option measure in the element layer table (141), the resource layer table (143) or the environment layer table (149) before processing advances to a software block 309.

The software in block 309 checks the bot date table (163) and deactivates causal predictive model bots with creation dates before the current system date. The software in block 309 then retrieves the information from the measure layer table (145), the common schema table (157), the element layer table (141), the transaction layer table (142), the resource layer table (143) and the environment layer table (149) in order to initialize causal predictive model bots for the measure or underlying source of uncertainty for an option measure in accordance with the frequency specified by the user (41) in the system settings table (162). Bots are independent components of the application software that complete specific tasks. In the case of causal predictive model bots, their primary task is to refine the performance driver selection to include only causal “drivers”. A series of predictive model bots are initialized at this stage because it is impossible to know in advance which predictive model will produce the “best” set of causal variables for each measure. The series for each measure or underlying source of uncertainty for an option measure includes a number of causal predictive model bot types: Bayesian, Granger, LaGrange, path analysis and Tetrad. Every causal predictive model bot contains the information shown in Table 21.

TABLE 21
1. Unique ID number (based on date, hour, minute, second of creation)
2. Creation date (date, hour, minute, second)
3. Mapping information
4. Storage location
5. Causal predictive model type
6. Subject entity
7. Node depth being modeled (1, 2, 3 or 4)
8. Function measure, underlying source of uncertainty for an option measure or component of context

After the causal predictive model bots are initialized by the software in block 309, the bots activate in accordance with the frequency specified by the user (41) in the system settings table (162). Once activated, they retrieve the data for the measure or underlying source of uncertainty for an option measure and sub-divide the variables into two sets, one for training and one for testing. After the causal predictive model bots complete their training for each model, the software in block 309 uses a model selection algorithm to identify the model that best fits the data. For the system of the present embodiment, a cross validation algorithm (e.g., the tenfold cross validation algorithm) is used for model selection. The drivers identified by the selected model are saved in the in the element layer table (141), the resource layer table (143) or the environment layer table (149) in the Resilient Contextbase (50) for possible inclusion in the final model before processing advances to a software block 311.

The software in block 311 determines if clustering improves the accuracy of the regression model for the measure or underlying source of uncertainty for an option measure for the subject entity (22). A primal graphical LASSO (dp-glasso) model is created for the overall measure or underlying source of uncertainty for an option measure, for each cluster and for each regime of data in accordance with the cluster and regime assignments identified by the bots in block 304. All of the primal graphical LASSO (dp-glasso) models use the best set of performance drivers identified in the prior stages of processing as inputs. The set of models that have the smallest amount of error after training as using the root mean squared error measure comprise the best set of models. Other error algorithms such as entropy measures may also be used. There are four possible outcomes from this analysis as shown in Table 22.

TABLE 22
1. A single model with no clustering
2. A plurality of models that are defined by temporal clustering (no variable clustering)
3. A plurality of models that are defined by variable clustering (no temporal clustering)
4. A plurality of models that are defined by temporal clustering and variable clustering

If the software in block 311 determines that clustering improves the accuracy of the regression models for the measure, then separate models for each cluster will be used in all subsequent analyses of the subject entity (22). Alternatively, if clustering does not improve the overall accuracy of the regression models for the subject entity (22), then a single overall model will be used in all subsequent processing. After the results of the analysis are stored in the measure layer table (145), processing advances to a software block 312.

The software in block 312 retrieves the information from the measure layer table (145), the common schema table (157), the element layer table (141), the transaction layer table (142), the resource layer table (143) and the environment layer table (149) in order to initialize measure model bots for the current measure. Bots are independent components of the application software that complete specific tasks. In the case of measure model bots, their primary task is to develop at least one model for the measure being evaluated that uses the best set of performance drivers as inputs. Measure model bots are always initialized for the overall measure. The results of the analysis in block 311 determine if bots will also be created for each cluster and/or for each regime of data in accordance with the cluster and regime assignments identified by the bots in block 304. The base measure model is a primal graphical LASSO (dp-glasso) model. A plurality of other predictive models including neural network, CART (classification and regression tree), graphical LASSO, projection pursuit regression, stepwise regression, linear regression, multivalent models, MARS (multivariate adaptive regression splines), power law, elastic net, ridge regression and generalized additive model (GAM) are also evaluated at this point. Every measure model bot contains the information shown in Table 23.

TABLE 23
1. Unique ID number (based on date, hour, minute, second of creation)
2. Creation date (date, hour, minute, second)
3. Mapping information
4. Storage location
5. Subject entity
6. Node depth being modeled (1, 2, 3 or 4)
7. Function measure, underlying source of uncertainty for an option measure or component of context
8. Predictive model type (elastic net, power law, graphical LASSO, etc.)
9. Type: overall, cluster, regime, cluster & regime

After measure model bots are initialized, the bots activate in accordance with the frequency specified by the user (41) in the system settings table (162). Once activated, the bots retrieve the specified data from the appropriate table in the Resilient Contextbase (50) and develop a measure model using the training methods detailed in FIG. 17 for each algorithm. After the measure model bots complete their training, the software in the block completes an analysis to determine if a transfer of learning between models developed using different algorithms improves the overall measure model accuracy. As shown in table 24 below, the primal graphical LASSO (dp-glasso) model is used as the base model and the software in the block completes an analysis to see if adding the element and factor inputs identified by any of the other algorithms including the causal predictive model algorithms from block 309 improves overall model accuracy.

TABLE 24

| Algorithm | Best fit element inputs: | Best fit factor inputs |
|---|---|---|
| Base Model-Dp-glasso | Elements A & B | Factors M & N |
| Linear regression | Elements A, B & C | Factors M, N & W |
| Neural network | Elements B, C & D | Factors N, W & Z |
| Test 1-Dp-glasso | Elements A, B & C | Factors M, N & W |
| Test 2-Dp-glasso | Elements A, B, & D | Factors M, N & Z |
| Test 3-Dp-glasso | Elements A, B, C & D | Factors M, N, W & Z |

While only five tests are shown in Table 24, it is to be understood that all possible combinations of the identified element variables and factor variables will be tested. After the identity of the best set of inputs for modeling the current function measure or underlying source of uncertainty for an option measure when using a primal graphical LASSO (dp-glasso) model are saved in the measure layer table (141), processing advances to a software block 313.

The software in block 313 uses sparse probabilistic principal component analysis to identify the contribution of each of the components of context (the inputs to the model) to the measure or underlying source of uncertainty (output) modeled by the software in block 312. After the contributions are identified and saved in the measure layer table (141), processing advances to a software block 314.

The software in block 314 checks the measure layer table (145) in the Resilient Contextbase (50) to see if the current model is a source of uncertainty for options based measure like contingent liabilities, real options or competitor risk. If the current model is not for a source of uncertainty for an options based measure, then processing returns to software block 301. When the software in block 301 determines that all measures and sources of uncertainty for option measures have current models for all node depths, then processing advances to software block 333. Alternatively, if the current model is for a source of uncertainty for an options based measure, then processing advances to a software block 315.

The software in block 315 retrieves the information from the measure layer table (145), the common schema table (157), the element layer table (141), the transaction layer table (142), the resource layer table (143) and the environment layer table (149) in order to initialize option model series bots for the current option measure. Bots are independent components of the application software in the present embodiment that complete specific tasks. In the case of option model series bots, their primary task is to develop a plurality of models for the value of the option measure. Each of the plurality of models uses the same inputs that are used in the primal graphical LASSO (dp-glasso) model for the source of uncertainty of the option. The baseline model for an option measure is comprised of the primal graphical LASSO (dp-glasso) model for the source of uncertainty for the option and a binomial option model that uses the output from the primal graphical LASSO (dp-glasso) model as an input. The baseline model is created by the software in block 315. A tournament of predictive model algorithms selected from the group consisting of neural network, CART (classification and regression tree), graphical LASSO, projection pursuit regression, stepwise regression, linear regression, multivalent models, MARS (multivariate adaptive regression splines), power law, elastic net, ridge regression and generalized additive model (GAM) are used at this point. The output from the model using each algorithm is compared to the output from the baseline model. The model with the lowest error as measured by the root mean squared algorithm is stored in the measure layer table (145) as the model for the option if the error of said model is below the maximum error rate for option series models specified by the user (41) in the system settings table (162). If the error of the best model from the tournament of predictive models is above the maximum error rate for option series models specified by the user (41) in the system settings table (162), then the baseline model is stored in the system settings table as the model for the option. After a model for the option has been stored, processing returns to software block 301. When the software in block 301 determines that all measures and sources of uncertainty for option measures have current models for all node depths, then processing advances to a software block 333.

The software in block 333 tests the performance drivers to see if there is interaction between elements, factors and/or resources by entity. The software in this block identifies interaction by evaluating a chosen model based on stochastic-driven pairs of performance driver sets (all the performance drivers for a single component of context comprise a set). If the accuracy of such a model is higher that the accuracy of statistically combined models trained on attribute subsets, then the attributes from subsets are considered to be interacting and then they form an interacting set. Other tests of driver interaction can be used to the same effect. The software in block 333 also tests the performance drivers to see if there are “missing” performance drivers that are influencing the results. If the software in block 333 does not detect any performance driver interaction or missing variables for each entity, then system processing advances to a software block 342. Alternatively, if missing data or performance driver interactions across elements, factors and/resources are detected by the software in block 333 for one or more measures, processing advances to a software block 334.

The software in block 334 evaluates the interaction between performance drivers in order to classify the performance driver set. The performance driver set generally matches one of seven patterns of interaction: a multi-component loop, a feed forward loop, a feed back loop (asynchronous or synchronous), a single input driver, a multi-input driver, auto-regulation or a chain. After classifying each performance driver set the software in block 334 prompts the user (41) via a structure revision window (706) to accept the classification and continue processing and/or adjust the specification(s) for the resilient context elements, resources and/or factors in some other way in order to minimize or eliminate interaction that was identified. For example, the user (41) can also choose to re-assign a performance driver to a new resilient context element or factor to eliminate an identified interdependency. After the optional input from the user (41) is saved in the element layer table (141), the resource layer table (143), the environment layer table (149) and the system settings table (162), system processing advances to a software block 335. The software in block 335 checks the element layer table (141), the resource layer table (143), the environment layer table (149) and system settings table (162) to see if there are any changes in structure. If there have been changes in the structure, then processing returns to software block 211 and the system processing described previously is repeated using the new structure. Alternatively, if there are no changes in structure, then the information regarding the element interaction provided by the user (41) is saved in the measure layer table (144) before processing advances to a software block 342.

The software in block 342 checks the resilience layer table (144) in the Resilient Contextbase (50) to determine if there are current resilience models for the subject entity (22) and the components of context for all node depths. If all resilience models are current, then processing advances to a software block 352. In the alternative, if all resilience models are not current, then processing advances to a software block 345. Table 25 below shows the type of resilience measures for the components of context that will be developed depending on the node depth specified in the system settings table (162).

TABLE 25

| Node | Number of models | | |
|---|---|---|---|
| depth | developed | Inputs | Output |
| 1 | Fourteen, one for each | Resilience indicators for each system | System Resilience |
| | system | | |
| 2 | Models from node | Resilience indicators for each organ | Organ Resilience |
| | depth 1, plus one for | | |
| | each organ | | |
| 3 | Models from node | Resilience indicators for each cell type | Cell Resilience |
| | depth 2, plus one for | | |
| | each cell type | | |

The software in block 345 retrieves the information from the measure layer table (145), the common schema table (157), the element layer table (141), the transaction layer table (142), the resource layer table (143) and the environment layer table (149) in order to initialize resilience history bots for either the subject entity or for one of the components of resilient context that exceeded the cutoff criteria for one or more periods for one or more clusters or regimes. Bots are independent components of the application software that complete specific tasks. In the case of resilience history bots, their primary tasks are to use the historical data to calculate the resilience measure for either the subject entity or for one of the components of resilient context that exceeded the cutoff criteria. It is worth noting at this point that the user (41) has the option of specifying the resilience measure in the system settings table (162). Every resilience history bot contains the information shown in Table 26.

TABLE 26
1. Unique ID number (based on date, hour, minute, second of creation)
2. Creation date (date, hour, minute, second)
3. Mapping information
4. Storage location
5. Selected resilience measure
6. Node depth being modeled (1, 2, 3 or 4)

After resilience history bots are initialized they activate in accordance with the frequency specified by the user (41) in the system settings table (162). Once activated, the bots retrieve data for the specified time periods from the appropriate table in the Resilient Contextbase (50) and analyze the data in order to calculate the selected resilience measure. The calculated resilience measures for the entity and each component of context are then saved in the resilience model table (166) before processing advances to a software block 346.

The software in block 346 develops the indicators that will be used to model entity resilience by learning from the data. There are up to six indicators of resilience in each model. The six indicators are selected from: an indicator of surplus component of context capacity, an indicator of effective redundancy, an indicator of entity stability, a pattern match frequency indicator, an indicator of the dynamic entropy of the components of resilient context and a performance driver diversity indicator as detailed below:
a) Surplus capacity for each of the components of context is an indicator of average component of context output and peak component of context output compared to the maximum output that can be produced by the component of context. For example, measures of epithelial progenitor cells are used to identify surplus capacity in the heart. There are three outputs from this analysis: the ratio of average output to maximum output for each component of context, the ratio of peak output to the maximum output for each component of context and an overall average=(average output+peak output) divided by (2 times maximum output) for each component of context. An overall average surplus capacity percentage is also calculated for all components of context.
b) Effective redundancy is a metric that accounts for the fact that alternative sources of receiving an input from a component of resilient context are generally not as efficient as the primary source for the input. Because the relative lack of efficiency can manifest itself as an increase in the time required to obtain the input or an increase in the amount of resources required to obtain the input, the effective redundancy considers the total amount of resources required to produce the same level of input for each time period by dividing the period output by the total amount of resources. For example, if there were two sources of the same input and both had the same efficiency, then the redundancy metric would be 2. In the case where there were two sources of the same input and one of the sources required twice as much time and twice as many resources to produce the same level of input, then the redundancy metric would be 1.25 (one plus (one/(two times two))).
c) Entity stability is measured using lyapunov exponents for the component of context function measure performance. The Lyapunov exponents are obtained by estimating the Lyapunov matrix using an average of several finite time approximations of the limit defining Lyapunov matrix.
d) Pattern match frequency is a metric that identifies the percentage of time any of the patterns in subject entity related data match patterns known to represent a decline in resilience and health. The system of the present embodiment includes a number of patterns that are known to represent a decline in resilience and health. These patterns include patterns of brain activity, gait and network dynamics. Pattern match frequency is identified using the two sliding windows algorithm.
e) The independence of the components of context is measured using a dynamic entropy measure for the components of context in the network of components of context that define the entity. The dynamic entropy measure used for this analysis comprises the Shannon entropy associated with each component of context.
f) The indicator of performance driver diversity is calculated by finding the smallest number of components of resilient context that are responsible for a combined total of 50% of the measure variability.

After the resilience indicators have been calculated and stored in the resilience layer table (144), processing advances to software blocks 304, 306, 308, 309, 311, 312 and 313 where the processing described previously is used to develop a resilience model and identify the set of resilience indicators that should be used for modeling the resilience of the subject entity (22) or the resilience of each component of resilient context. After this processing is complete, system processing advances to a software block 347.

The software in block 347 retrieves the information from the resilience layer table (144), the measure layer table (145), the common schema table (157), the element layer table (141), the transaction layer table (142), the resource layer table (143) and the environment layer table (149) in order to initialize resilience model bots for the current measure. Bots are independent components of the application software that complete specific tasks. In the case of resilience model bots, their primary task is to develop a resilience model for the entity or component of context being evaluated that uses the resilience measures as inputs and the resilience history as an output. The base resilience measure model is a primal graphical LASSO (dp-glasso) model. A plurality of predictive model algorithms including neural network, CART (classification and regression tree), graphical LASSO, projection pursuit regression, stepwise regression, linear regression, multivalent models, MARS (multivariate adaptive regression splines), elastic net, power law, ridge regression and generalized additive model (GAM) are used at this point. Every resilience model bot contains the information shown in Table 27.

TABLE 27
1. Unique ID number (based on date, hour, minute, second of creation)
2. Creation date (date, hour, minute, second)
3. Mapping information
4. Storage location
5. Node depth being modeled (1, 2, 3 or 4)
6. Cluster or regime
7. Type: overall, cluster, regime, cluster & regime

After resilience model bots are initialized, the bots activate in accordance with the frequency specified by the user (41) in the system settings table (162). Once activated, the bots retrieve the specified data from the appropriate table in the Resilient Contextbase (50) and develop a resilience model using the training methods detailed in FIG. 17 for each algorithm. After the resilience model bots complete their training, the software in the block completes an analysis to determine if a transfer of learning between models developed using different algorithms improves the overall resilience model accuracy. As shown in Table 28 below, the primal graphical LASSO (dp-glasso) model is used as the base model and the software in the block completes an analysis to see if adding the element and factor inputs identified by any of the other algorithms improves overall model accuracy. While only two other algorithms, neural net and linear regression, are shown, it is to be understood that the measures identified by all algorithms identified in the description of block 347 are used.

TABLE 28

| Algorithm | Best fit resilience measures |
|---|---|
| Base Model - | Element A surplus capacity, Average component entropy |
| Dp-glasso | |
| Linear | Resource G effective redundancy, Element B - Factor N |
| regression | entropy |
| Neural network | Subject entity stability, Element C - Resource H entropy |
| Test 1 - | Element A surplus capacity, Average component |
| Dp-glasso | entropy and Entity stability |
| Test 2 - | Entity stability, Element C - Resource H entropy and |
| Dp-glasso | Element A surplus capacity |

While only four tests are shown in Table 28, it is to be understood that all possible combinations of the identified resilience measures will be tested. After the identity of the best set of inputs for modeling the resilience of the entity or component of context using a primal graphical LASSO (dp-glasso) model are saved in the resilience layer table (144), processing advances to a software block 348.

The software in block 348 checks the system settings table (162) to see if physical models are going to be used to calibrate the resilience models. If they are not going to be used, then processing returns to software block 342. In the alternative, if physical models are going to be used to calibrate resilience models, then the software in block 348 checks physical model library (174) in the Resilient Contextbase (50) to determine if there is a physical model for the entity or component of context that is being modeled. If there is no physical model for the entity or component of context that is being modeled, then processing returns to software block 342. If there is a physical model for the entity or component of context that is being modeled, then processing advances to a software block 349.

The software in block 349 retrieves the physical model that corresponds to the entity or component of context that is being modeled from the physical model library (174). Some of the physical models included in the library are shown below in Table 29.

TABLE 29

| Models: | Description |
|---|---|
| “ns-3” - network simulator | a discrete-event network simulator for |
| | Internet systems |
| “Disim” - highway simulator | a lightweight microscopic highway traffic |
| | simulator |
| “CVSim” - heart simulator | a lumped-parameter model of the human |
| | cardiovascular system |

The software in block 349 uses the same data that was used to develop the resilience model for the entity or component of context that is being modeled to complete a simulation using the physical model. The software in block 349 then identifies any calibrations that may be needed to bring the resilience model in line with the physical model. A tournament of predictive model algorithms selected from the group consisting of primal graphical LASSO (dp-glasso), neural network, CART (classification and regression tree), projection pursuit regression, stepwise regression, linear regression, elastic net, multivalent models, MARS (multivariate adaptive regression splines), power law, graphical LASSO, ridge regression and generalized additive model (GAM) are used at this point to identify the relationship between the resilience model developed by the software in block 347 and the resilience pattern identified by the physical model. The model that produces the lowest error is combined with the previously developed resilience model to comprise a series model for resilience. The definition of the series model is added to the resilience layer table (144) in the resilience contextbase (50) before processing returns to software block 342. Once processing returns to software block 342, the software in the block checks to see if the resilience models are current for the subject entity (22) and for all the components of context. If all resilience models are not current, then processing returns to a software block 345 and the process described above is repeated. In the alternative, if all resilience models are current, then processing advances to software block 352.

The software in block 352 checks the bot date table (163) and deactivates event risk bots with creation dates before the current system date. The software in the block then retrieves the information from the transaction layer table (142), the resilience layer table (144), the event risk table (156), the common schema table (157) and the system settings table (162) in order to initialize event risk bots in accordance with the frequency specified by the user (41) in the system settings table (162). Bots are independent components of the application software that complete specific tasks. In the case of event risk bots, their primary tasks are to forecast the frequency and magnitude of entity events that are associated with negative measure performance in the resilience layer table (144). Entity events are events that have an impact on entity measure performance or component of context output that are not global events. The system of the present embodiment uses the Resilient Context Forecast Service (603) for event risk frequency and impact forecasts. Other forecasting methods can be used to the same effect. Every event risk bot contains the information shown in Table 30.

TABLE 30

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Node depth being modeled (1, 2, 3 or 4) |
| 6. | Event (transaction, action etc.) |

After the event risk bots are initialized they activate in accordance with the frequency specified by the user (41) in the system settings table (162). After being activated the bots retrieve the specified data and forecast the frequency and measure impact of the event risks. The resulting forecasts are stored in the event risk table (156) before processing advances to a software block 353.

The software in block 353 checks the bot date table (163) and deactivates extreme value bots with creation dates before the current system date. The software in block 353 then retrieves the information from the transaction layer table (142), the resilience layer table (144), the event risk table (156), the common schema table (157) and the system settings table (162) in order to initialize extreme value bots in accordance with the frequency specified by the user (41) in the system settings table (162). Bots are independent components of the application software that complete specific tasks. In the case of extreme value bots, their primary task is to forecast the extreme values for the drivers of the components of context, extreme values for the drivers of the subject entity and extreme values for entity event risks. The extreme value bots use the peak over threshold method to identify extreme driver values and extreme subject entity event risks. Other extreme value algorithms such as the blocks maxima method can be used to the same effect. The mapping information is then used to identify the elements, factors, resources and/or actions that will be affected by each extreme risk. Every extreme value bot activated in this block contains the information shown in Table 31.

TABLE 31

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Node depth being modeled (1, 2, 3 or 4) |
| 6. | Driver or entity event risk |

After the extreme value bots are initialized, they activate in accordance with the frequency specified by the user (41) in the system settings table (162). Once activated, they retrieve the specified information and identify extreme driver values. The extreme entity event risk information is stored in the scenarios table (168) in the Resilient Contextbase (50) before processing advances to a software block 354.

The software in block 354 checks the bot date table (163) and deactivates scenario bots with creation dates before the current system date. The software in block 354 then retrieves the information from the system settings table (162), the element layer table (141), the transaction layer table (142), the resource layer table (143), the resilience layer table (144), the environment layer table (149), the event risk table (156) and the common schema table (157) in order to initialize scenario bots in accordance with the frequency specified by the user (41) in the system settings table (162). Bots are independent components of the application software of the present embodiment that complete specific tasks. In the case of scenario bots, their primary task is to identify likely scenarios for the evolution of the element, factor and resource drivers and event risks by subject entity. The likely scenarios are developed by combining data that was previously obtained from other systems and data that was previously developed by the system of the present embodiment as shown in Table 32.

TABLE 32

| Sources of data: | Normal scenario | Extreme scenario |
|---|---|---|
| Global events | External databases | External databases |
| Subject entity events | Values from block 352 | Extreme values from |
| | | block 353 |
| Drivers | Driver values from | Extreme driver values |
| | best fit models | from block 353 |

A blended scenario could also be created that consists of the simple average of the normal and extreme value for each driver and/or event. Every scenario bot activated in this block contains the information shown in Table 33.

TABLE 33

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Type: normal or extreme |
| 6. | Driver or event |
| 7. | Subject entity or component of context |
| 8. | Measure |

After the scenario bots are initialized, they activate in accordance with the frequency specified by the user (41) in the system settings table (162). Once activated, they retrieve the specified information and develop the scenarios. After the scenario bots complete their processing, they save the resulting scenarios in the scenarios table (168) by entity in the contextbase (50) and processing advances to a software block 355.

The software in block 355 checks the bot date table (163) and deactivates measure relevance bots with creation dates before the current system date. The software in block 355 then retrieves the information from the system settings table (162) and the measure layer table (145) in order to initialize a bot for each subject entity being analyzed. Bots are independent components of the application software of the present embodiment that complete specific tasks. In the case of measure relevance bots, their task is to determine the relevance of each of the different function measures to the subject entity mission measure. The relevance of the measures is determined by using a series of predictive models to find the best fit relationship between the function measures and entity mission measure levels. The system of the present embodiment uses several different types of predictive models to identify the best fit relationship: primal graphical LASSO (dp-glasso), neural network, CART (classification and regression tree), projection pursuit regression, graphical LASSO, generalized additive model (GAM), MARS (multivariate adaptive regression splines), elastic net, linear regression, and stepwise regression. The coefficient of determination is used to identify the best fit model. Other methods of identifying the best fit model may also be used. Every measure relevance bot contains the information shown in Table 34.

TABLE 34

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Subject entity |
| 6. | Function measure(s) |
| 7. | Mission measure |

After the measure relevance bots are initialized by the software in block 355 they activate in accordance with the frequency specified by the user (41) in the system settings table (162). After being activated, the bots retrieve information and complete the analysis of the measure relevance. The relative measure contributions to the mission measure are saved in the measure layer table (145) by entity before processing advances to a software block 356.

The software in block 356 checks the system settings table (162) to see if the subject entity (22) being modeled is an extended subject entity. If the subject entity (22) being modeled is an extended subject entity, then processing advances to a software block 358. If the subject entity (22) being models is not an extended subject entity (22), then processing advances to a software block 357.

The software in block 357 checks the bot date table (163) and deactivates simulation bots with creation dates before the current system date. The software in block 357 then retrieves the information from the resilience layer table (144), the measure layer table (145), the event risk table (156), the common schema table (157), the system settings table (162) and the scenarios table (168) in order to initialize simulation bots in accordance with the frequency specified by the user (41) in the system settings table (162). Bots are independent components of the application software that complete specific tasks. In the case of simulation bots, their primary task is to complete multi-period simulations of subject entity (22) measure performance. The simulation bots run probabilistic multi-period simulations of measure performance using the normal scenario and the extreme scenario. They also run an unconstrained genetic algorithm simulation that evolves to the most negative value possible over the specified time period. In one embodiment, Monte Carlo models are used to complete the probabilistic simulation. However, other probabilistic simulation models such as Quasi Monte Carlo, genetic algorithm and Markov Chain Monte Carlo can be used to the same effect. Every simulation bot activated in this block contains the information shown in Table 35.

TABLE 35

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Type: normal, extreme, user specified or genetic algorithm |
| 6. | Time periods |
| 7. | Measure |
| 8. | Subject entity |

After the simulation bots are initialized, they activate in accordance with the frequency specified by the user (41) in the system settings table (162). Once activated, they retrieve the specified information and simulate measure performance by entity over the time periods specified by the user (41) in the system settings table (162) until the simulations converge on a solution. In doing so, the bots will forecast the range of values that can be expected for the specified measure by subject entity (22) for each scenario. The bots also create a summary of the overall risks facing the entity for the current measure by comparing the measure levels from the best fit model with the range of measure levels identified during simulation. Identifying the magnitude of risk from a single period simulation using the general method described above is straightforward as the measure level from the best fit measure model is compared to the range of values that are identified in the simulations that incorporate event risks and driver variability. In a multi-period simulation identifying the magnitude of risk is more complex as the biggest differential in magnitude from the best fit model value during any of the time periods modeled is the calculated risk as illustrated by the example shown in Table 36. The biggest differential in terms of percentage could also be used to the same effect.

TABLE 36

| Measure values | | Period 1 | Period 2 | Measured Risk |
|---|---|---|---|---|
| Best fit model | | 100 | 150 | |
| Normal Scenario | Highest | 90 | 145 | (10) |
| | Average | 80 | 130 | (20) |
| | Lowest | 60 | 120 | (40) |
| Extreme Scenario | Highest | 65 | 120 | (35) |
| | Average | 60 | 100 | (50) |
| | Lowest | 50 | 75 | (75) |

After the simulation bots complete their calculations, the resulting forecasts and risk measures are saved in the scenarios table (168) by entity and the risk summary is saved in the report table (153) in the Resilient Contextbase (50) before processing advances to a software block 359.

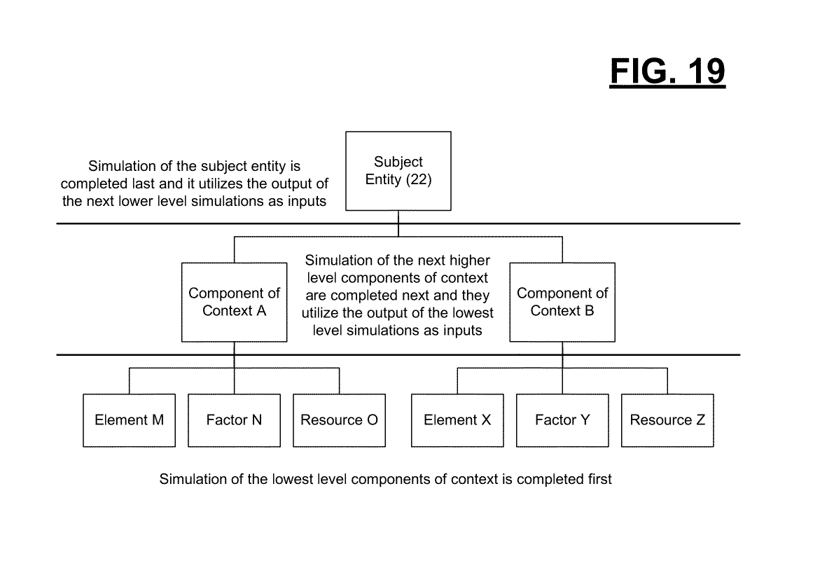

The software in block 358 checks the bot date table (163) and deactivates extended entity simulation bots with creation dates before the current system date. The software in block 358 then retrieves the information from the resilience layer table (144), the measure layer table (145), the event risk table (156), the common schema table (157), the system settings table (162) and the scenarios table (168) in order to initialize extended entity simulation bots in accordance with the frequency specified by the user (41) in the system settings table (162). Bots are independent components of the application software that complete specific tasks. In the case of extended entity simulation bots, their primary task is to complete multi-period simulations of the components of context output and the subject entity (22) measure performance by level. The levels in the extended entity are defined by the depth cutoff for the extended subject entity model input by the user (41) in the system settings table (162). Simulation starts at the lowest level and moves up until it reaches the subject entity level which is the top level. The results from the lower levels of simulation comprise inputs to the higher levels of simulation. FIG. 19 provides an overview of the order of completion for simulation by level for an extended subject entity. The extended entity simulation bots run probabilistic multi-period simulations of component of context output and measure performance using the normal scenario and the extreme scenario. They also run an unconstrained genetic algorithm simulation that evolves to the most negative value possible over the specified time period. In one embodiment, Monte Carlo models are used to complete the probabilistic simulation; however other probabilistic simulation models such as Quasi Monte Carlo, genetic algorithm and Markov Chain Monte Carlo can be used to the same effect. Every simulation bot activated in this block contains the information shown in Table 37.

TABLE 37

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Type: normal, extreme, user specified or genetic algorithm |
| 6. | Time periods |
| 7. | Measure |
| 8. | Subject entity or component of context |
| 9. | Level |

After the extended entity simulation bots are initialized, they activate in accordance with the frequency specified by the user (41) in the system settings table (162). Once activated, they retrieve the specified information and simulate component of context output and measure performance over the time periods specified by the user (41) in the system settings table (162) until the simulations converge on a solution. In doing so, the bots will forecast the range of performance and risk that can be expected for the specified measure or output by subject entity (22) for each scenario. The bots also create a summary of the overall risks facing the entity for the current measure by comparing the measure levels from the best fit model with the range of measure levels identified during simulation. After the extended entity simulation bots complete their calculations, the resulting forecasts are saved in the scenarios table (168) by entity and the risk summary is saved in the report table (153) in the Resilient Contextbase (50) before processing advances to a software block 359.

The software in block 359 checks the bot date table (163) and deactivates mission simulation bots with creation dates before the current system date. The software in block 359 then retrieves the information from the resilience layer table (144), the measure layer table (145), the event risk table (156), the common schema table (157), the system settings table (162) and the scenarios table (168) in order to initialize mission simulation bots in accordance with the frequency specified by the user (41) in the system settings table (162). Bots are independent components of the application software that complete specific tasks. In the case of mission simulation bots, their primary task is to complete multi-period simulations of subject entity (22) mission measure levels. The simulation bots run probabilistic multi-period simulations of measure levels using the output from the function measure simulations completed under the normal, extreme and/or user defined scenarios. They also run an unconstrained genetic algorithm simulation that evolves to the most negative value possible over the specified time period. In one embodiment, Monte Carlo models are used to complete the probabilistic simulation. However, other probabilistic simulation models such as Quasi Monte Carlo, genetic algorithm and Markov Chain Monte Carlo can be used to the same effect. Every simulation bot activated in this block contains the information shown in Table 38.

TABLE 38

| | |
|---|---|
| 1. | Unique ID number (based on date, hour, minute, second of creation) |
| 2. | Creation date (date, hour, minute, second) |
| 3. | Mapping information |
| 4. | Storage location |
| 5. | Type: normal, extreme, user specified or genetic algorithm |
| 6. | Time periods |
| 7. | Mission Measure |
| 8. | Subject entity |

After the mission simulation bots are initialized, they activate in accordance with the frequency specified by the user (41) in the system settings table (162). Once activated, they retrieve the specified information and simulate mission measure levels over the time periods specified by the user (41) in the system settings table (162) until the simulations converge on a solution. In doing so, the bots will forecast the range of values that can be expected for the specified mission measure by subject entity (22) for each scenario. The same bots use the time period specified by the user (41) for sustainability analyses. If the mission measure values drop below a required level during one or more of the simulated time periods, then the bots will note the fact that the subject entity survival may be at risk. After the results of the mission simulation are saved in the measure layer table (145), processing advances to a software block 360.

The software in block 360 checks the bot date table (163) and deactivates context frame bots with creation dates before the current system date. The software in block 360 then retrieves the information from the element layer table (141), the transaction layer table (142), the resource layer table (143), the resilience layer table (144), the measure layer table (145), the environment layer table (149), the reference layer table (154), the common schema table (157) and the system settings table (162) in order to initialize context frame bots in accordance with the frequency specified by the user (41) in the system settings table (162). Bots are independent components of the application software that complete specific tasks. In the case of context frame bots, their primary task is to define a context frame for the subject entity (22) for each of the mission measures that have been specified and store them in the resilient context frame table (160). After the context frames are defined, the software in block 360 displays details regarding each context frame to the user (41) via the frame definition window (709). The user (41) has the option of modifying the definition of the one or more of the context frames and of specifying one or more sub-context frames. The modifications to the context frames and the subcontext frame definitions are stored in the resilient context frame table (160) before processing advances to a software block 371.

The software in block 371 checks the system settings table (162) to see if a return on resilience analysis is going to be completed. If a return on resilience analysis is not going to be completed, then processing advances to a software block 374. If a return on resilience analysis is going to be completed, then processing advances to a software block 372.

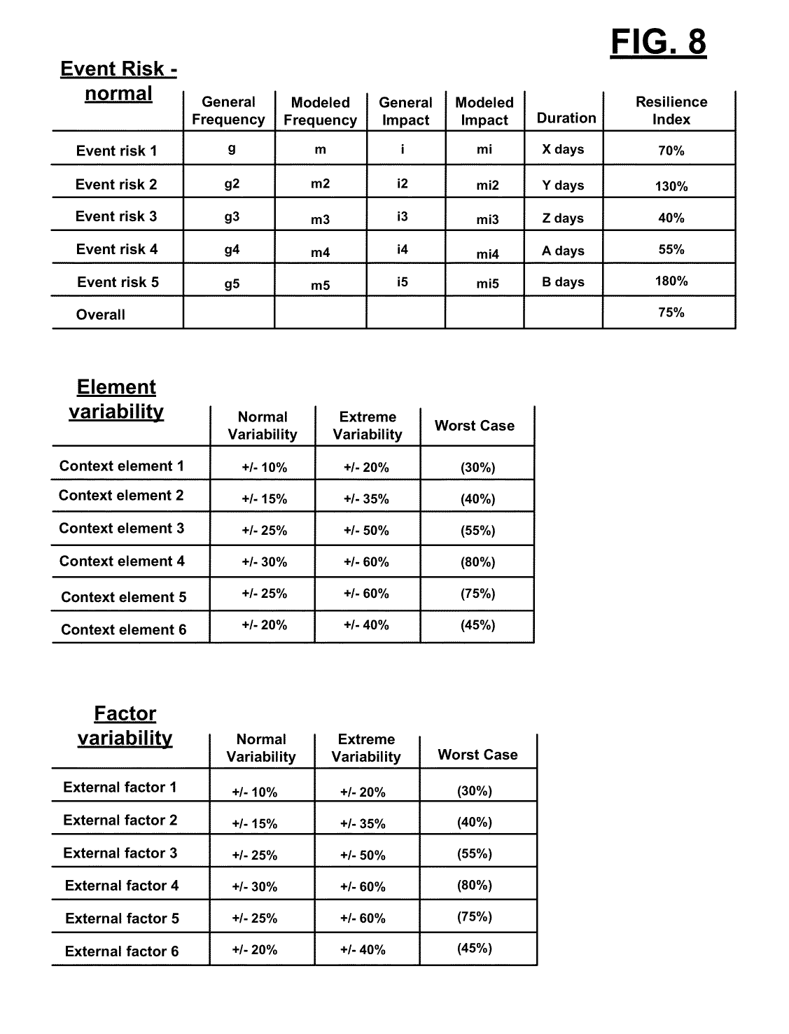

The software in block 372 displays a summary of the calculated risks for each measure and scenario using the format shown in FIG. 8 using a resilience feature window (716). The format shown in FIG. 8 can also be used to show overall risks for an entity where the risks for each measure are multiplied by the measure relevance to determine overall impact of the different risks on the subject entity (22). For brevity sake, the event risks are only shown for one scenario—normal. It should be understood that the event risk information would generally be displayed for the normal, extreme and worst case scenarios. The displayed event risk information combines the event frequency and impact identified previously with the data for each of the scenarios to calculate the modeled frequency and modeled impact for each of a plurality of event risks under each scenario. As is well known in the art, global event risks are often transferred to others using insurance policies or securities such as catastrophe bonds so there is generally information available about the frequency and impact (e.g., $ loss, function loss, duration, etc.) that may result from each event. The resilience index compares the expected total impact of an event to the global impact of the same event for others by dividing the product of the modeled frequency, impact and duration with the global frequency, impact and duration of the event. Event risks with a resilience index above 100% are those where the entity experiences greater losses than generally would be expected. While those below 100% are those where the experienced losses are expected to be less severe than the losses suffered by others in a similar situation. An overall resilience index is also calculated based on the weighted impact of the events over the next year. The element and factor variability portions of the display shown in FIG. 8 rely on data obtained from the simulations completed under each scenario by the software in block 357 or block 358.

The software in block 372 prompts the user (41) via the resilience features window (716) to specify one or more resilience features (also referred to as actions) that will improve the resilience of the entity by: specifying one or more actions that will reduce the impact of one or more event risks for one or more scenarios, specifying one or more actions that will reduce the frequency of one or more event risks for one or more scenarios, specifying one or more actions that will reduce element variability for one or more scenarios, specifying one or more actions that will reduce factor variability for one or more scenarios, specifying one or more actions that will reduce resource variability for one or more scenarios and/or specifying one or more actions that will improve resilience by increasing subject entity redundancy, increasing surplus capacity, reducing the percentage of time the entity experiences negative patterns, increasing subject entity stability and/or maintain independence between components of context. For example, a backup generator with a fuel supply could be purchased to increase redundancy. The increased redundancy will reduce the impact of power outages caused by natural disasters for a business entity. In a similar manner a microbiome supplement could be used to reduce the impact of a virus for an individual. The specified actions will include the cost and time associated with such actions as well as a mapping of the expected impact of the specified actions on the event risks, element drivers, factor drivers and/or resource drivers. These data are saved in the scenarios table (168) for use in optimization calculations. After data storage is complete, processing advances to a software block 373.

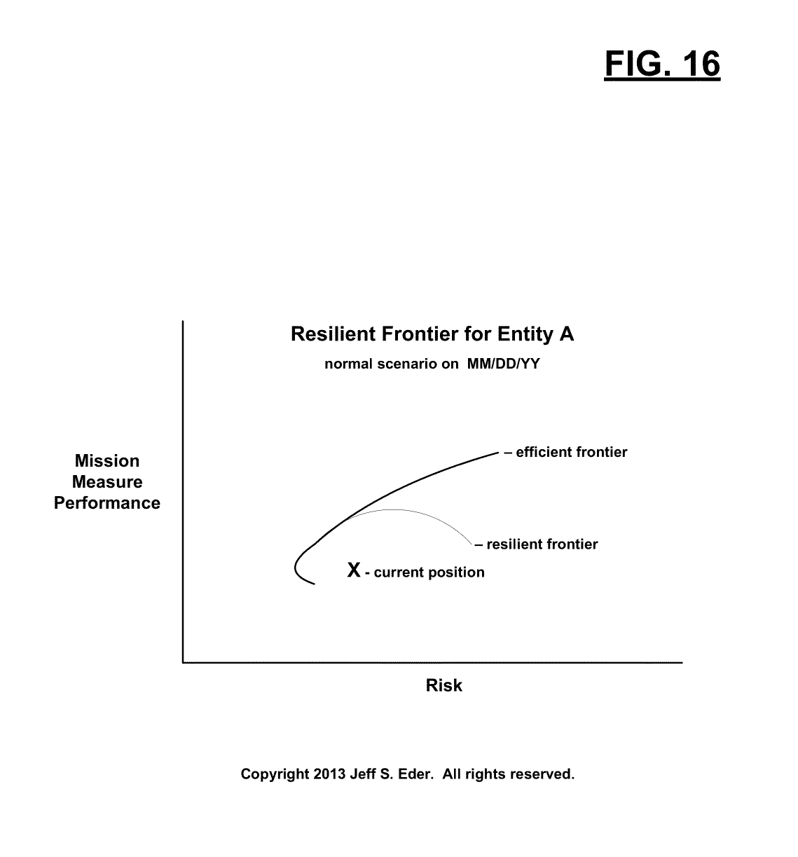

The software in block 373 uses the list of potential actions saved in the scenarios table (168) and their mapped impacts to forecast the function measure and mission measure levels under one or more scenarios. The list of potential actions and their simulated impacts comprise a swarm. The best set of resilience actions are then identified using particle swarm optimization. A comparison of the subject entity measures (e.g., value or survival time period) before and after taking the best set of resilience actions can be used to calculate the return on resilience. The return on resilience calculation also incorporates the reduced need for risk transfer expenditures after resilience actions are implemented. For example, the calculated improvement in the value of a firm after implementing the optimal set of resilience actions and reducing expenditures for risk transfer can be divided by the cost of the resilience actions (also referred to as resilience programs) to calculate a return on resilience. Particle swarm optimization also identifies the resilient frontier by identifying the best set of resilience actions for each level of risk as shown in FIG. 16. After the best set of resilience actions, the resilient frontier and the return on resilience are saved in the resilience layer table (144), processing advances to a software block 374.

The software in block 374 takes the previously stored schema from the common schema table (157) and combines it with the relationship information in the measure layer table (145) to develop the entity ontology. The ontology is then stored in the ontology table (152) using the OWL language. Use of the RDF (resource description framework) based OWL language will enable the communication and synchronization of the entities ontology with other entities and will facilitate the extraction and use of information from the semantic web. The semantic web rule language (swrl) that combines OWL with Rule ML can also be used to store the ontology. After the relevant entity ontology is saved in the Resilient Contextbase (50), processing advances to a software block 402.

### Resilient Context Service Propagation

The flow diagrams in FIG. 13A and FIG. 13B detail the processing that is completed by the portion of the application software (400) that identifies the valid resilient context space, identifies principles, integrates the different contexts into an overall resilient context, propagates a plurality of Resilient Context Services, optionally manages the operation of one or more devices and optionally displays and prints management reports detailing the measure performance and resilience of an entity. Processing in this portion of the application software (400) starts in software block 402.

The software in block 402 calculates expected uncertainty by multiplying the user (41) and subject matter expert (42) estimates of narrow system (4) uncertainty by the relative importance of the data from the narrow system for each function measure. The expected uncertainty for each measure is expected to be lower than the actual uncertainty (measured using R² as discussed previously) because total uncertainty is a function of data uncertainty plus parameter uncertainty (e.g., are the specified elements, resources and factors the correct ones) and model uncertainty (does the model accurately reflect the relationship between the data and the measure). After saving the uncertainty information in the uncertainty table (150) processing advances to a software block 403.

The software in block 403 retrieves information from the resilience layer table (144), the measure layer table (145) and the resilient context frame table (160) in order to define the valid resilient context space for the current relationships and measures stored in the Resilient Contextbase (50). The current measures and relationships are compared to previously stored resilient context frames to determine the range of contexts in which they are valid with the confidence interval specified by the user (41) in the system settings table (162). The resulting list of valid frame definitions stored in the resilient context space table (151). The software in this block also completes a stepwise elimination of each user specified constraint. This analysis helps determine the sensitivity of the results and may indicate that it would be desirable to use some resources to relax one or more of the established constraints. The results of this analysis are stored in the resilient context space table (151) before processing advances to a software block 410.

The software in block 410 integrates the one or more entity contexts into an overall entity resilient context using the weightings specified by the user (41) or the weightings developed over time from user preferences. This overall resilient context and the one or more separate contexts are propagated as a SOAP compliant Entity Resilience System (30). Each layer is presented separately for each function and the overall resilient context. As discussed previously, it is possible to bundle or separate layers in any combination. This information in the service is communicated to the Resilient Context Suite (625), narrow systems (4) and devices (3) using a Resilient Context Service Interface window (711) before processing passes to a software block 414. It is to be understood that the system is also capable of bundling this the resilient context information by layer in one or more bots as well as propagating a layer containing this information for use in a computer operating system, mobile operating system, network operating system or middleware application.

The software in block 414 checks the system settings table (162) in the Resilient Contextbase (50) to determine if a natural language interface window (714) is going to be used. If a natural language interface is going be used, then processing advances to a software block 420. Alternatively, if a natural language interface is not going to be used, then processing advances to a software block 431.

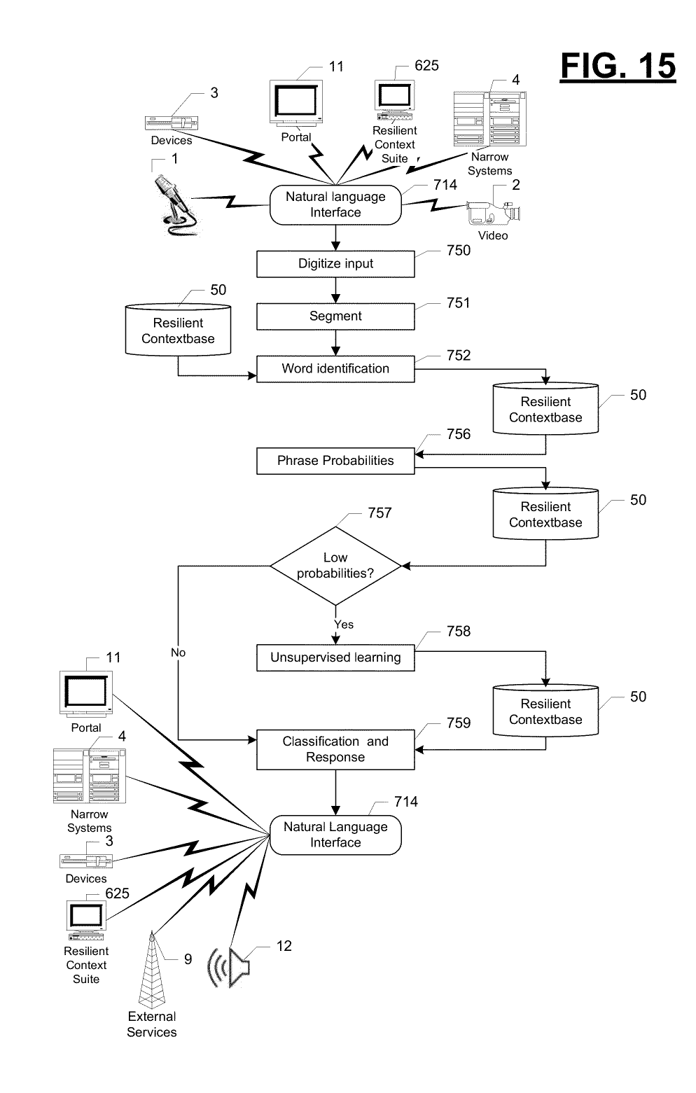

The software in block 420 combines the ontology developed in prior steps in processing with unsupervised natural language processing to provide a true natural language interface to the Entity Resilience System (30). A true natural language interface is an interface that provides the system of the present embodiment with an understanding of the meaning of the words as well as a correct identification of the words. As shown in FIG. 15, the processing to support the development of a true natural language interface starts with the receipt of audio input to the natural language interface window (714) from audio sources (1), video sources (2), devices (3), narrow systems (4), a portal (11) and/or services in the Resilient Context Suite (625). From there, the audio input passes to a software block 750 where the input is digitized in a manner that is well known. After being digitized, the input passes to a software block 751 where it is segmented into phonemes using a constituent-resilient context model. The phonemes are then passed to a software block 752 where they are compared to previously stored phonemes in the phoneme table (170) to identify the most probable set of words contained in the input. The most probable set of words are saved in the natural language table (169) in the Resilient Contextbase (50) before processing advances to a software block 756. The software in block 756 compares the word set to previously stored phrases in the phrase table (172) and the ontology from the ontology table (152) to classify the word set as one or more phrases. After the classification is completed and saved in the natural language table (169), processing passes to a software block 757.

The software in block 757 checks the natural language table (169) to determine if there are any phrases that could not be classified with a weight of evidence level greater than or equal to the level specified by the user (41) in the system settings table (162). If all the phrases could be classified within the specified levels, then processing advances to a software block 759. Alternatively, if there were phrases that could not be classified within the specified levels, then processing advances to a software block 758.

The software in block 758 uses the constituent-resilient context model that uses word classes in conjunction with a dependency structure model to identify one or more new meanings for the low probability phrases. These new meanings are compared to known phrases in an external database (7) such as the Penn Treebank and the system ontology (152) before being evaluated, classified and presented to the user (41). After classification is complete, processing advances to software block 759.

The software in block 759 uses the classified input and ontology to generate a response (that may include the completion of actions) to the translated input and generate a response to the natural language interface (714) that is then forwarded to a device (3), a narrow system (4), an external service (9), a portal (11), an audio output device (12) or a service in the Resilient Context Suite (625). This process continues until all natural language input has been processed. When this processing is complete, processing advances to a software block 431. The software in block 431 checks the system settings table (162) in the Resilient Contextbase (50) to determine if services or bots are going to be created. If services or bots are not going to be created, then processing advances to a software block 434. Alternatively, if services or bots are going to be created, then processing advances to a software block 432.

The software in block 432 supports a development interface window (712) that supports four distinct types of development projects by the Resilient Context Programming System (610):

programming devices (3) with rules of behavior for different resilient contexts that are consistent with the resilient context frame being provided—e.g., when in church (reference layer location), do not ring unless it is the boss (element) calling;

the development of extensions to Resilient Context Suite (625) in order to provide the user (41) with the specific information for a given requirement;

the development of Resilient Context Bots (650) to complete one or more actions, initiate one or more actions, complete one or more events, respond to requests for actions, respond to actions, respond to events, obtain data or information and combinations thereof. The software developed using this option can be used for software bots or agents and robots; and

the development of new resilient context aware services.

The first screen displayed by the Resilient Context Programming System (610) asks the user (41) to identify the type of development project. The second screen displayed by the Resilient Context Programming System (610) will depend on which type of development project the user (41) is completing. If the first option is selected, then the user (41) is given the option of using pre-defined patterns and/or patterns extracted from existing narrow systems (4) to modify one or more of the services in the Resilient Context Suite (625). The user (41) can also program the service extensions using C++ or Java with or without the use of patterns. If the second option is selected, then the user (41) is shown a display of the previously developed common schema (157) for use in defining an assignment and resilient context frame for a Resilient Context Bot (650).

After the assignment specification is stored in the bot assignment table (167), the Resilient Context Programming System (610) defines a probabilistic simulation of bot performance under the three previously defined scenarios. The results of the simulations are displayed to the user (41) via the development interface window (712). The Resilient Context Programming System (610) then gives the user (41) the option of modifying the bot assignment or approving the bot assignment. If the user (41) decides to change the bot assignment, then the change in assignment is saved in the bot assignment table (167) and the process described for this software block is repeated. Alternatively, if the user (41) does not change the bot assignment, then Resilient Context Programming System (610) completes two primary functions. First, it combines the bot assignment with results of the simulations to develop the set of program instructions that will maximize bot performance under the forecast scenarios. The bot programming includes the entity ontology and is saved in the bot assignment table (167). In one embodiment Prolog is used to program the bots. Prolog is used because it readily supports the situation calculus analyses used by the Resilient Context Bots (650) to evaluate their situation and select the appropriate course of action. Each Resilient Context Bot (650) has the ability to interact with bots and entities that use other schemas or ontologies in an automated fashion. If the third option is selected, then the previous information about the resilient context quotient for the device (3) is developed and used to select the pre-programmed options (e.g., ring, don't ring, silent ring, etc.) that will be presented to the user (41) for implementation. The user (41) will also be given the ability to construct new rules for the device (3) using the parameters contained within the device-specific resilient context frame. If the fourth option is selected, then the user (41) is given a pre-defined resilient context frame interface shell along with the option of using pre-defined patterns and/or patterns extracted from existing narrow systems (4) to develop a new service. The user (41) can also program the new service completely using C#, Python or Java. When programming is complete using one of the four options, processing advances to software block 434.

The software in block 434 prompts the user (41) via a report display and selection data window (713) to review and select reports for printing. The format of the reports is either graphical, numeric or both depending on the type of report the user (41) specified in the system settings table (162). If the user (41) selects any reports for printing, then the information regarding the selected reports is saved in the report table (153). After the user (41) has finished selecting reports, the selected reports are displayed to the user (41) via the report display and selection data window (713). After the user (41) indicates that the review of the reports has been completed, processing advances to a software block 435. The processing can also pass to block 435 if the maximum amount of time to wait for no response specified by the user (41) in the system settings table is exceeded before the user (41) responds.

The software in block 435 checks the report table (153) to determine if any reports have been designated for printing. If reports have been designated for printing, then processing advances to a software block 436. It should be noted that in addition to standard reports like a performance risk matrix and the graphical depictions of the efficient frontier shown (FIG. 16), the system of the present embodiment can generate reports that rank the elements, factors, resources and/or risks in order of their importance to function measure performance and/or measure risk by entity, by measure and/or for the entity as a whole. The system can also produce reports that compare results to plan for actions, impacts and measure performance if expected performance levels have been specified and saved in appropriate resilient context layer. The software in block 436 sends the designated reports to the printer (118). After the reports have been sent to the printer (118), processing advances to a software block 438. Alternatively, if no reports were designated for printing, then processing advances directly from block 435 to block 438. The software in block 438 checks the system settings table (162) to determine if the system is operating in a continuous run mode. If the system is operating in a continuous run mode, then processing returns to block 222 and the processing described previously is repeated in accordance with the frequency specified by the user (41) in the system settings table (162). Alternatively, if the system is not running in continuous mode, then the processing advances to a software block 439 where the system stops.

### Individualized Medicine System

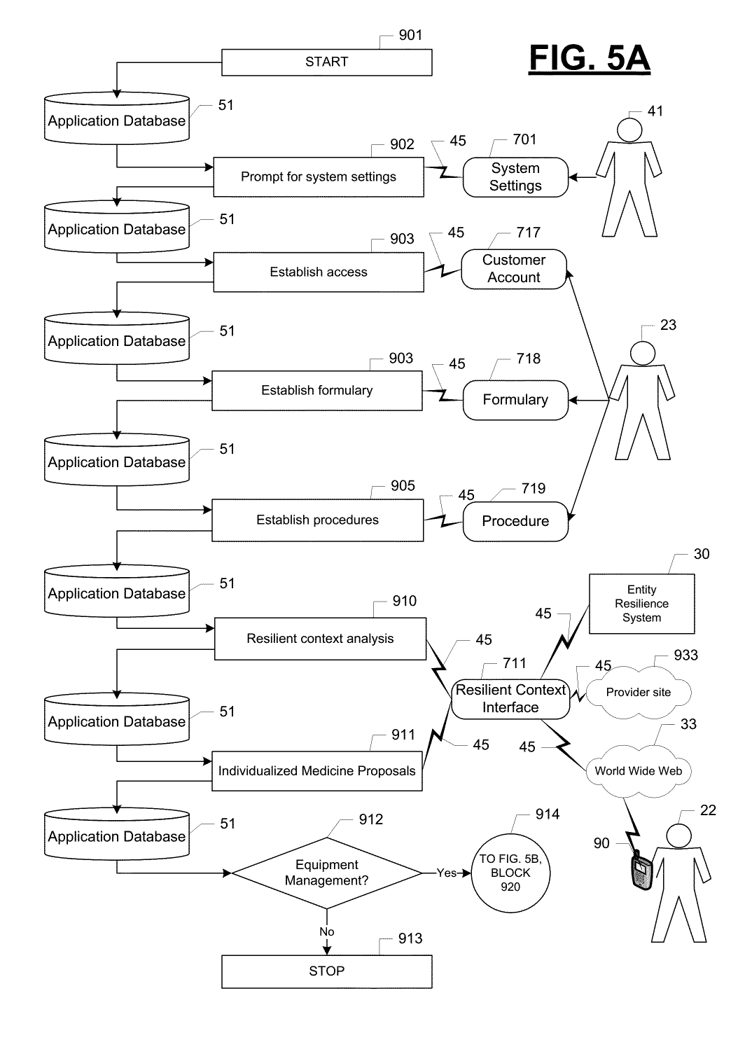

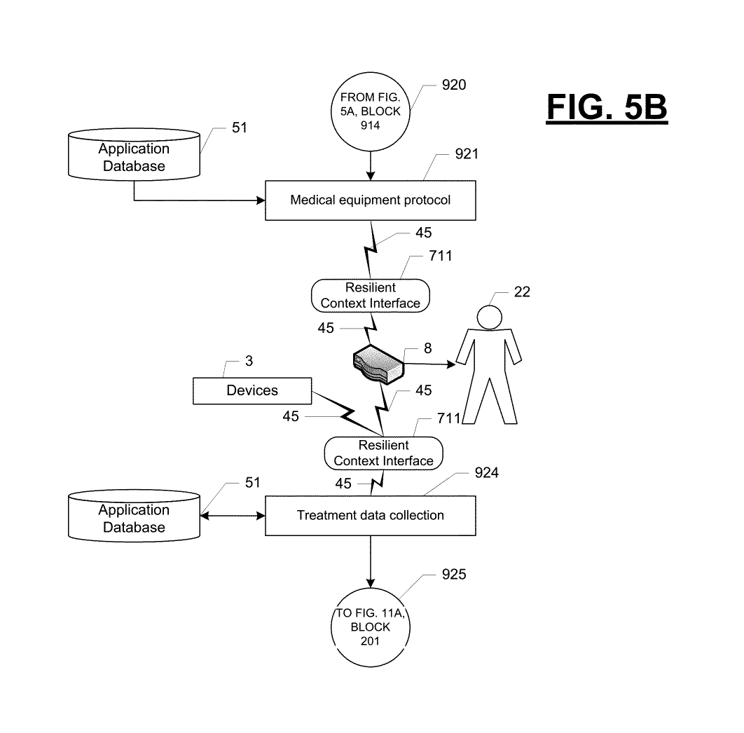

The flow diagrams in FIG. 5A and FIG. 5B detail the processing by the Individualized Medicine System (100) required to obtain the information that supports the development, identification and/or provision of individualized medicine services that are appropriate to the resilient context of a specific subject entity (22).

Processing in this portion of the Individualized Medicine System (100) starts in a software block 901 which immediately passes processing to a software block 902. The software in block 902 prompts the user (41) via a system settings data window (701) to provide a plurality of system setting information. The system setting information is stored in a system settings table (560) in the application database (51) in a manner that is well known. The specific inputs the user (41) is asked to provide at this point in processing are shown in Table 39.

TABLE 39

| | |
|---|---|
| 1. | Metadata standard (XML or RDF) |
| 2. | Base currency for all pricing |
| 3. | Source of conversion rates for currencies |
| 4. | Manage medical equipment performance? (If yes, specify equipment |
| | and type of protocol) |
| 5. | Use similarity measures for search? (default is “No”) |

After the storage of system setting data are complete, processing advances to a software block 903. The software in block 903 prompts each medical service provider (23) via a customer account window (717) to establish an account and/or to open an existing account in a manner that is well known. For existing medical service providers (23), account information is obtained from a customer account table (561). New medical service providers (23) have their new information stored in the customer account table (561). After the medical service provider (23) has established access to the system, processing advances to a software block 905.

The software in block 905 prompts each medical service provider (23) via a formulary window (718) to describe the medication protocols and/or treatment protocols that will be made available to individual subject entities. Each medical service provider (23) also identifies the elements of resilient context that are affected by the medication or treatment protocols and the equipment that may be used as part of the delivery of the medication protocol or treatment protocol (e.g., infusion pump for medication or fluid delivery, a medical linear accelerator for Intensity Modulated Radiotherapy etc.). The Individualized Medicine System (100) supports the use to medication and treatment protocols that are based any combination of different aspects of the subject entity's resilient context. Table 40 below provides some illustrative examples.

TABLE 40

| Resilient context | | |
|---|---|---|
| aspect(s) considered | Type of protocol | Example |
| Indexed subject entity | Protocol varies with heart | 5 mg/day of amlodipine if heart resilience is high, 2.5 |
| resilience | resilience index | mg/day of amlodipine if heart resilience is low |
| | classification | |
| Subject entity | Protocol varies with heart | 5 mg/day of amlodipine if heart resilience measure is |
| resilience measure | resilience measure | above 0.9, dosage drops linearly to 2.5 mg/day when |
| | | heart resilience measure is 0.4 or below |
| Presence of one or | Protocol varies with | 50 mg/25 mL of doxorubicin per day when epithelial |
| more context | presence/absence of | progenitor cell concentration exceeds .05%; 20 |
| elements | biomarker elements of | mg/10 mL of doxorubicin per day when epithelial |
| | context | progenitor cell concentration is below .05%; |
| Subject entity | Protocol varies with | Cathartic dosage determined by resilience level is |
| resilience value plus | resilience measure | cut in half in tropical climates (defined by the Tropic |
| reference frame | value and location | of Cancer in the northern hemisphere at |
| value (location) | | approximately 23.4378° N and the Tropic of |
| | | Capricorn in the southern hemisphere at 23.4378° S) |

The data regarding the formulary is stored in the formulary table (562) in the application database (51). After storage of the formulary data are complete, processing advances to a software block 907.

The software in block 907 prompts each medical service provider (23) via a procedure window (719) to define procedures that can be provided to one or more subject entities (22) of an entity resilience system (30) that is linked to the Individualized Medicine System (100). There are four different types of procedures that can be specified by a medical service provider (23)—additions, corrections, maintenance and removal. Table 41 shows more details about the different types of procedures that can be specified for an offering.

TABLE 41

| Type of | |
|---|---|
| procedure | Information Provided |
| Addition | Name of addition to the subject entity, element(s) of context affected by the |
| | addition to the subject entity, expected affect of addition on subject entity |
| | components of context, time required to complete addition, expense required to |
| | complete addition, entities that are required to complete addition procedure, |
| | procedures that are typically completed at the same time, medications that are |
| | typically provided at the same time, procedures that generally cannot be |
| | completed at the same time and medications that generally cannot be used at the |
| | same time |
| Correction | Name of correction to the subject entity, element(s) of context affected by the |
| | correction, expected affect of correction on subject entity components of context, |
| | time required to complete correction, expense required to complete correction, |
| | entities that are required to complete correction procedure, procedures that are |
| | typically completed at the same time, medications that are typically provided at |
| | the same time, procedures that generally cannot be completed at the same time |
| | and medications that generally cannot be used at the same time. |
| Maintenance | Name of maintenance procedure, element(s) of context affected by the |
| | maintenance procedure, expected effect of maintenance on subject entity |
| | components of context, time required to complete maintenance, expense required |
| | to complete maintenance, entities that are required to complete maintenance |
| | procedure, procedures that are typically completed at the same time, medications |
| | that are typically provided at the same time, procedures that generally cannot be |
| | completed at the same time and medications that generally cannot be used at the |
| | same time |
| Removal | Element(s) of context removed from the subject entity, expected affect of removal |
| | on subject entity components of context, time required to complete removal, |
| | expense required to complete removal, entities that are required to complete |
| | removal procedure, procedures that are typically completed at the same time, |
| | medications that are typically provided at the same time, procedures that |
| | generally cannot be completed at the same time and medications that generally |
| | cannot be used at the same time as the removal procedure |

Each medical service provider (23) also identifies the elements of resilient context that are affected by the procedure. The system can also obtain offer information from networks and entities that are not medical service providers if it is made available on the Internet in XML or RDF format, via an API or some other means. The data regarding the procedures are stored in the procedures table (563) in the application database (51). After data storage is complete, processing advances to a software block 910.

The software in block 910 retrieves information from the Resilient Contextbase (51) that defines the resilient context of the subject entity (22) and stores it in a resilient context table (569) in the application database (50). The software in block 910 then combines said information with the procedures (563) and formulary (562) previously stored by the medical service providers (23) in order to complete a plurality of multi-level simulations using the Resilient Context Optimization Service (604). The simulations identify one or more combinations of medication protocols, treatment protocols and/or procedures that are expected to improve the health of the subject entity (22). An optimal combination of said protocols and procedures that defines the resilient frontier for subject entity health is also identified. The results of these simulations are saved in the impact summary table (566) in the application database (50). Proposals are prepared for transmission to the subject entity for each procedure, each treatment and each medication that was identified as being part of the one or more combinations before processing advances to a software block 911.

The software in block 911 provides one or more medical service provider sites (933) on the World Wide Web (33) with proposals regarding medication and/or procedures as appropriate for the resilient context of each subject entity (22) via the resilient context interface window (711) that establishes and maintains a connection with each medical service provider site (933) in a manner that is well known. As part of its processing, the software in block 911 may call on one or more services in the Resilient Context Suite (625). Information about the delivery of medication proposals to each subject entity (22) is saved in a medication proposal table (564). Information about the delivery of procedure proposals to each subject entity (22) is saved in a procedure proposal table (565). Information about the acceptance of medication proposals and the delivery of medication to each subject entity (22) is saved in a medication delivery table (567). Information about the acceptance of procedure proposals and the delivery of procedures to each subject entity (22) is saved in a procedure delivery table (568). The information from these tables can then used to prepare a bill for each subject entity (22). The monthly totals are saved in the customer account table (561). Resilient contexts that were associated with a delivery will be captured and stored in the resilient context table (569) for dissemination to one or more medical service providers (23). This information will enable medical service providers (23) to better identify resilient contexts that are appropriate for specific medication protocols, treatment protocols and/or procedures. After this processing completes, system processing advances to a software block 912.

The software in block 912 checks the system settings table (560) to see if a piece of medical equipment (8) is going to be managed in accordance with the resilient context for the subject entity that was stored in the resilient context table (569). If medical equipment (8) is not going to be managed, then processing advances to a software block 513 where processing stops. If medical equipment (8) is going to be managed, then processing advances to a software block 921.

The software in block 921 checks the system settings table (560) to determine which type of medical equipment (8) is going to be managed and the type of protocol that is going to be used. The software in block 921 retrieves the medication protocol or treatment protocol from the formulary table (562), converts the protocol to an appropriate machine readable form and transmits the protocol to the medical equipment (8) via the resilient context interface window (711) before processing advances to a software block 924.

The software in block 924 collects data from the medical equipment (8) and any device (3) that is monitoring the subject entity (22) during treatment, converts said data as required and then transmits said data to the entity resilience system. The processing described previously is then used to identify any changes to the resilient context of the subject entity (22). If changes to the resilient context generate a need for a change in the protocol being administered, the changes will be identified and transmitted to the medical equipment (8) in an automated fashion.

While the above description contains many specificities, these should not be construed as limitations on the scope of the invention, but rather as an exemplification of one embodiment thereof. Accordingly, the scope of the invention should be determined not by the embodiment illustrated, but by the appended claims and their legal equivalents.

1. A non-transitory computer readable storage medium that stores one or more programs, the one or more programs comprising instructions for an individualized medicine system comprised of a medication delivery device; at least one computer with at least one processor having circuitry to execute instructions; a storage device available to the at least one processor with the one or more programs stored therein, which when executed:
accept an input that defines or selects a subject entity and a plurality of measures for said subject entity, a node depth for an extended subject entity model and a formulary;
prepare a plurality of subject entity related data for processing;
transform at least a portion of said data into a resilience model, the extended subject entity model and a resilient context for the subject entity where the resilient context comprises the extended subject entity model and the resilience model;
identify a protocol for a medication from the formulary that is appropriate for the resilient context of the subject entity; and
configure the medication delivery device to deliver said medication in accordance with the protocol;
wherein the plurality of measures comprise a health measure, one or more function measures and a resilience measure and wherein the resilient context further comprises a measure layer comprised of one or more function measure models, a function measure relevance model and one or more other context layers selected from the group consisting of: element, resource, environment, reference and transaction.

2. The system of claim 1, wherein the medication delivery device comprises an infusion pump.

3. The system of claim 1, wherein the formulary comprises:
a description of one or more medication protocols that are available to the subject entity;
a description of one or more treatment protocols that are available to the subject entity;
an identification of one or more elements of the resilient context that are affected by each of the medication protocols;
an identification of the one or more elements of the resilient context that are affected by each of the treatment protocols;
an identification of medical equipment used to support the delivery of each of the medication protocols; and
an identification of medical equipment used to support the delivery of each of the treatment protocols.

4. The system of claim 1, wherein the resilience measure comprises either: (1) an amount of time required to return to a level of measure performance that is within a specified percentage of an average level that was being experienced by the subject entity before a negative event; or (2) a negative event magnitude that is required to decrease the measure performance of the subject entity by more than a defined percentage.

5. The system of claim 1, wherein the one or more resilience models each comprise a regression model of the resilience measure that identifies a contribution of one or more resilience indicators to a resilience of a component of the subject entity's resilient context where the resilience model of each component of context is calibrated by comparing its output with the results of a physical model simulation and where the resilience indicators are selected from the group consisting of effective redundancy, driver diversity percentage, surplus capacity, entity stability, pattern match frequency and component independence.

6. The system of claim 1, wherein developing the extended subject entity model comprises:
analyzing a plurality of data from a ribosome profiling system; and
analyzing a plurality of high throughput screening data using a sequence alignment algorithm and a sequence analysis tool where the sequence alignment algorithm is selected from the group consisting of Short Oligonucleotide Analysis Package algorithm, Bowtie, Basic Local Alignment Search Tool (BLAST), Blast Like Alignment Tool (BLAT), Burrows-Wheeler Aligner (BWA), FANSe, Genomemapper, Mapping and Assembly with Quality (MAQ), RNA Sequence Analysis Pipeline and Short Read Mapping Package (SHRiMP) and where the sequence analysis tool is selected from the group consisting of ANNOVAR, BEDTools and the genome analysis tool kit (GATK).

7. The system of claim 1, wherein the sequences of instructions further cause the at least one processor to: use the resilient context of the subject entity to complete one or more activities selected from the group consisting of customize a treatment for the subject entity, customize a test for the subject entity, order a treatment for the subject entity, order a test for the subject entity, forecast a sustainable longevity for the subject entity, analyze an impact of a user specified change on the one or more subject entity measures, simulate the subject entity's measures, establish a priority for one or more actions, establish an expected measure level for the subject, identify and display a resilient frontier for one or more of the subject entity's measures and identify and display a set of data that is most relevant to the subject.

8. A non-transitory computer readable storage medium that stores one or more programs, the one or more programs comprising instructions for an individualized medicine system comprised of a medication delivery device; at least one computer with at least one processor having circuitry to execute instructions; a storage device available to the at least one processor with the one or more programs stored therein, which when executed:
accepting an input that defines or selects a subject entity and a plurality of measures for said subject entity, a node depth for an extended subject entity model and a formulary;
preparing a plurality of subject entity related data for processing;
transforming at least a portion of said data into one or more resilience models, the extended subject entity model and a resilient context for the subject entity where the resilient context comprises the extended subject entity model, a resilience layer comprised of the one or more resilience models, a measure layer comprised of one or more function measure models, a function measure relevance model and one or more other context layers selected from the group consisting of element, resource, environment, reference and transaction;
using the resilient context of the subject entity to complete one or more activities selected from the group consisting of customize a treatment for the subject entity, customize a test for the subject entity, order a treatment for the subject entity, order a test for the subject entity, forecast a sustainable longevity for the subject entity, analyze an impact of a user specified change on the one or more subject entity measures, simulate the subject entity's measure levels, forecast an expected measure level for the subject entity, identify and display a set of data that is most relevant to the subject entity, identify and display one or more medication or treatment protocols from the formulary that are optimal for the resilient context of the subject entity by completing a multi-period simulation, identify and display a resilient frontier for one or more of the subject entity's measures and manage a piece of medical equipment wherein the transformation of at least part of the data into the extended subject entity model comprises: developing a primal graphical LASSO predictive model of the subject entity health measure that outputs a contribution of one or more components of context to a value of the health measure;
determining a contribution from each of one or more components of context to the health measure; and
developing a predictive model for each of the components of context where at least one of the components of context comprises a microbiome.

9. The non-transitory computer readable storage medium of claim 8, wherein the formulary comprises:
a description of one or more medication protocols that are available to the subject entity;
a description of one or more treatment protocols that are available to the subject entity;
an identification of one or more elements of the resilient context that are affected by each of the medication protocols;
an identification of the one or more elements of the resilient context that are affected by each of the treatment protocols;
an identification of medical equipment used to support the delivery of each of the medication protocols; and
an identification of medical equipment used to support the delivery of each of the treatment protocols.

10. The non-transitory computer readable storage medium of claim 8, wherein the resilience measure is either: (1) an amount of time required to return to a level of measure performance that is within a specified percentage of an average level that was being experienced by the subject entity before a negative event; or (2) a negative event magnitude that is required to decrease the measure performance of the subject entity by more than a defined percentage.

11. The non-transitory computer readable storage medium of claim 8, wherein the one or more resilience models each comprise a primal graphical LASSO regression model of the resilience measure that identifies a contribution of one or more resilience indicators to a resilience of a component of the subject entity's resilient context where the resilience indicators are selected from the group consisting of effective redundancy, driver diversity percentage, surplus capacity, entity stability, pattern match frequency and component independence.

12. The non-transitory computer readable storage medium of claim 8, wherein developing the extended subject entity model comprises analyzing a plurality of data from a ribosome profiling system, analyzing a plurality of high throughput screening data using a sequence alignment algorithm and a sequence analysis tool where the sequence alignment algorithm is selected from the group consisting of Short Oligonucleotide Analysis Package algorithm, Bowtie, Basic Local Alignment Search Tool (BLAST), Blast Like Alignment Tool (BLAT), Burrows-Wheeler Aligner (BWA), FANSe, Genomemapper, Mapping and Assembly with Quality (MAQ), RNA Sequence Analysis Pipeline and Short Read Mapping Package (SHRiMP) and where the sequence analysis tool is selected from the group consisting of ANNOVAR, BEDTools and the genome analysis tool kit (GATK).

13. The non-transitory computer readable storage medium of claim 8, wherein the transformation of at least part of the data into the extended subject entity model comprises:
developing a primal graphical LASSO predictive model of the subject entity health measure that outputs a contribution of one or more components of context to a value of the health measure;
determining a contribution from each of one or more components of context to the health measure; and
developing a predictive model for each of the components of context where at least one of the components of context comprises a microbiome.

14. A method, comprising: at a computing device having one or more processors, a connection to a medication delivery device and memory storing one or more programs executed by the one or more processors to perform the method:
accepting an input that defines or selects a subject entity and a plurality of measures for said subject entity, a node depth for an extended subject entity model and a formulary;
preparing a plurality of subject entity related data for processing;
transforming at least a portion of said data into a resilience model, the extended subject entity model and a resilient context for the subject entity where the resilient context comprises the extended subject entity model and the resilience model;
identifying a protocol for a medication from the formulary that is appropriate for the resilient context of the subject entity; and
configuring the medication delivery device to deliver said medication in accordance with the protocol;
wherein the plurality of measures comprise a health measure, one or more function measures and a resilience measure and wherein the resilient context further comprises a measure layer comprised of one or more function measure models, a function measure relevance model and one or more other context layers selected from the group consisting of: element, resource, environment, reference and transaction.

15. The method of claim 14, wherein the medication delivery device comprises an infusion pump.

16. The method of claim 14, wherein the formulary comprises:
a description of one or more medication protocols that are available to the subject entity;
a description of one or more treatment protocols that are available to the subject entity;
an identification of one or more elements of the resilient context that are affected by each of the medication protocols;
an identification of the one or more elements of the resilient context that are affected by each of the treatment protocols;
an identification of medical equipment used to support the delivery of each of the medication protocols; and
an identification of medical equipment used to support the delivery of each of the treatment protocols.

17. The method of claim 14, wherein the resilience measure comprises either: (1) an amount of time required to return to a level of measure performance that is within a specified percentage of an average level that was being experienced by the subject entity before a negative event; or (2) a negative event magnitude that is required to decrease the measure performance of the subject entity by more than a defined percentage.

18. The method of claim 14, wherein the one or more resilience models each comprise a regression model of the resilience measure that identifies a contribution of one or more resilience indicators to a resilience of a component of the subject entity's resilient context where the resilience model of each component of context is calibrated by comparing its output with the results of a physical model simulation and where the resilience indicators are selected from the group consisting of effective redundancy, driver diversity percentage, surplus capacity, entity stability, pattern match frequency and component independence.

19. The method of claim 14, wherein developing the extended subject entity model comprises:
analyzing a plurality of data from a ribosome profiling system; and
analyzing a plurality of high throughput screening data using a sequence alignment algorithm and a sequence analysis tool where the sequence alignment algorithm is selected from the group consisting of Short Oligonucleotide Analysis Package algorithm, Bowtie, Basic Local Alignment Search Tool (BLAST), Blast Like Alignment Tool (BLAT), Burrows-Wheeler Aligner (BWA), FANSe, Genomemapper, Mapping and Assembly with Quality (MAQ), RNA Sequence Analysis Pipeline and Short Read Mapping Package (SHRiMP) and where the sequence analysis tool is selected from the group consisting of ANNOVAR, BEDTools and the genome analysis tool kit (GATK).

20. The method of claim 14, wherein the sequences of instructions further cause the at least one processor to: use the resilient context of the subject entity to complete one or more activities selected from the group consisting of customize a treatment for the subject entity, customize a test for the subject entity, order a treatment for the subject entity, order a test for the subject entity, forecast a sustainable longevity for the subject entity, analyze an impact of a user specified change on the one or more subject entity measures, simulate the subject entity's measures, establish a priority for one or more actions, establish an expected measure level for the subject, identify and display a resilient frontier for one or more of the subject entity's measures and identify and display a set of data that is most relevant to the subject.
